(19) United States
(12) Patent Application Publication
(10) Pub. No.: US 2003/0139364 A1
(43) Pub. Date: Jul. 24, 2003

(54) Methods and products for enhancing immune responses using imidazoquinoline compounds

(75) Inventors: Arthur M. Krieg, Wellesley, MA (US); Christian Schetter, Hilden (DE); Robert L. Bratzler, Concord, MA (US); Jorg Vollmer, Dusseldorf (DE); Marion Jurk, Dusseldorf (DE); Stefan Bauer, Muenchen (DE)

Correspondence Address: WOLF GREENFIELD & SACKS, PC, FEDERAL RESERVE PLAZA, 600 ATLANTIC AVENUE, BOSTON, MA, 02210-2211, US

(73) Assignee: University of Iowa Research Foundation, Iowa City, IA

(21) Appl. No.: 10/272,502

(22) Filed: Oct. 15, 2002

Related U.S. Application Data
(60) Provisional application No. 60/329,208, filed on Oct. 12, 2001.

(51) Int. Cl. A61K 48/00; A61K 38/00; G01N 33/53; A61K 31/56; A61K 31/522; A61K 31/4745

(52) U.S. Cl. 514/44; 514/292; 514/263.38; 514/171; 435/7.1; 514/2

(57) ABSTRACT

The invention involves administration of an imidazoquinoline agent in combination with another therapeutic agent. The combination of drugs may be administered in synergistic amounts or in various dosages or at various time schedules. The invention also relates to kits and compositions concerning the combination of drugs. The combinations can be used to enhance ADCC, stimulate immune responses and/or patient and treat certain disorders.

### RELATED APPLICATIONS

This application claims priority to U.S. Provisional Patent Application filed Oct. 12, 2001 entitled “METHODS AND PRODUCTS FOR ENHANCING IMMUNE RESPONSES USING IMIDAZOQUINOLINE COMPOUNDS”, Serial No. 60/329,208, the contents of which are incorporated by reference herein in their entirety.

### BACKGROUND OF THE INVENTION

Cancer is the second leading cause of death, resulting in one out of every four deaths, in the United States. In 1997, the estimated total number of new diagnoses for lung, breast, prostate, colorectal and ovarian cancer was approximately two million. Due to the ever increasing aging population in the United States, it is reasonable to expect that rates of cancer incidence will continue to grow.

Asthma is a chronic inflammatory disease effecting 14-15 million persons in the United States alone.

Infectious disease is one of the leading causes of death throughout the world. In the United States alone the death rate due to infectious disease rose 58% between 1980 and 1992. During this time, the use of anti-infective therapies to combat infectious disease has grown significantly and is now a multi-billion dollar a year industry. Even with these increases in anti-infective agent use, the treatment and prevention of infectious disease remains a challenge to the medical community throughout the world.

The immunostimulatory capacity of a variety of immunostimulatory nucleic acids has been well documented. Depending upon their nature and composition and administration, immunostimulatory nucleic acids are capable of inducing T helper 1 (Th1) responses, of suppressing T helper 2 (Th2) responses, and in some instances, inducing Th2 responses.

Imidazoquinoline agents have similarly been reported to possess immunomodulatory activity, including the ability to activate B lymphocytes, induce interferon alpha (IFN-alpha) production, and upregulate tumor necrosis factor (TNF), interleukin 1 (IL-1) and interleukin 6 (IL-6). The utility of imidazoquinoline agents in the treatment of viral infections and tumors has also been suggested.

### SUMMARY OF THE INVENTION

The invention is based, in part, on the finding that when imidazoquinoline agents are used in conjunction with other therapeutic agents, such as antibodies, immunostimulatory nucleic acids, antigens, C8-substituted guanosines, and disorder-specific medicaments, some unexpected and improved results are observed. For instance, the efficacy of the combination of imidazoquinoline agents and the other therapeutic agent is profoundly improved over the use of either compound alone.

The results are surprising, in part, because the imidazoquinoline agents and the other therapeutic agents in some instances act through different mechanisms and would not necessarily be expected to improve the efficacy of the other in a synergistic manner.

In one aspect, the invention provides a method for stimulating antibody-dependent cellular cytotoxicity (ADCC) in a subject. The method comprises administering an antibody and an agent selected from the group consisting of an imidazoquinoline agent and an C8-substituted guanosine to a subject in need of such treatment in an amount effective to stimulate antibody dependent cellular cytotoxicity in the subject. In some embodiments, the amount effective to stimulate antibody dependent cellular cytotoxicity is a synergistic amount.

In one embodiment, the imidazoquinoline agent is administered prior to the antibody. In another embodiment, the antibody is selected from the group consisting of an anti-cancer antibody, an anti-viral antibody, an anti-bacterial antibody, an anti-fungal antibody, an anti-allergen antibody, and an anti-self antigen antibody. In related embodiments, the subject has or is at risk of having a disorder selected from the group consisting of asthma/allergy, infectious disease, cancer and warts.

The following embodiments apply to this and other aspects of the invention.

In one embodiment, the agent is imidazoquinoline agent. In another embodiment, both the imidazoquinoline agent and the C8-substituted guanosine are administered to the subject. C8-substituted guanosines can be selected from the group consisting of 8- mercaptoguanosine, 8-bromoguanosine, 8-methylguanosine, 8-oxo-7,8-dihydroguanosine, C8-arylamino-2′-deoxyguanosine, C8-propynyl-guanosine, C8- and N7- substituted guanine ribonucleosides such as 7-allyl-8-oxoguanosine (loxoribine) and 7-methyl-8-oxoguanosine, 8-aminoguanosine, 8-hydroxy-2′-deoxyguanosine, and 8-hydroxyguanosine.

In some embodiments in which the imidazoquinoline agent is administered to the subject, the subject is further administered a poly-arginine. In other embodiments, interferon-alpha (e.g., Intron A) is administered to the subject.

In one embodiment, the imidazoquinoline agent is an imidazoquinoline amine. In another embodiment, the imidazoquinoline agent is selected from the group consisting of imiquimod/R-837, S-28463/R-848 (Resiquimod), imidazoquinoline amines, imidazopyridine amines, 6,7-fused cycloalkylimidazopyridine amines, 1,2 bridged imidazoquinoline amines, and 4-amino-2ethoxymethyl-alpha, alpha-dimethyl-1H-imidazo[4,5-c]quinolines-1-ethanol.

In still other embodiments, the method further comprises administering an immunostimulatory nucleic acid to the subject. In certain embodiments, the agent is administered prior to the immunostimulatory nucleic acid. The immunostimulatory nucleic acid may be selected from the group consisting of a CpG nucleic acid and a poly-G nucleic acid. In certain embodiments, the immunostimulatory nucleic acid is selected from the group consisting of a poly-T nucleic acid, a T-rich nucleic acid, a TG nucleic acid, a CpI nucleic acid and a methylated CpG nucleic acid. In some embodiments, the immunostimulatory nucleic acid has a backbone modification. The backbone modification may be selected from the group consisting of a phosphorothioate modification and a peptide modification (such as for example a morpholino backbone modification), but is not so limited. In one embodiment, the immunostimulatory nucleic acid has a backbone that is chimeric. In still another embodiment, the immunostimulatory nucleic acid is a nucleic acid that is free of CpG, T-rich or poly-G motifs. In some embodiments, the immunostimulatory nucleic acid with a phosphorothioate modified backbone is free of a CpG motif, a T-rich motif or a poly-G motif. The immunostimulatory nucleic acid may be a nucleic acid which stimulates a Th1 immune response. In some embodiments, the immunostimulatory nucleic acid which stimulates a Th1 immune response is not a CpG nucleic acid. In other embodiments, the immunostimulatory nucleic acid which stimulates a Th1 immune response is not a T-rich nucleic acid.

In another embodiment, the method further comprises exposing the subject to an antigen. The antigen may be selected from the group consisting of a tumor antigen, a viral antigen, a bacterial antigen, a parasitic antigen, and a fungal antigen.

In another aspect, the invention provides a method for modulating an immune response in a subject. The method comprises administering to a subject in need of such treatment an immunostimulatory nucleic acid and an agent selected from the group consisting of an imidazoquinoline agent and an C8-substituted guanosine in an amount effective to modulate the immune response. In one embodiment, the amount effective to modulate the immune response is a synergistic amount. In an important embodiment, the imidazoquinoline agent is administered prior to the immunostimulatory nucleic acid. In certain embodiments, the immunostimulatory nucleic acid is a CpG nucleic acid. In other embodiments, the immunostimulatory nucleic acid has a nucleotide sequence of (#2006) TCG TCG TTT TGT CGT TTT GTC GTT (SEQ ID NO:1).

In one embodiment, modulating an immune response means inducing a Th1 immune response. In another embodiment, the immune response is a Th1 immune response. In another embodiment, the immune response involves antibody dependent cellular cytotoxicity. In another embodiment, the immune response is an innate immune response. In some embodiments, the immune response is a local immune response, while in other embodiments, the immune response is a systemic immune response. In certain embodiments, the immune response is a mucosal immune response.

In this and other embodiments of the invention, the method further comprises administering a disorder-specific medicament to the subject. The disorder-order specific medicament may be selected from the group consisting of a cancer medicament, an asthma/allergy a medicament, an infectious disease medicament, and a wart medicament. The anti-microbial medicament may be selected from the group consisting of an anti-bacterial agent, an anti-viral agent, an anti-fungal agent, and an anti-parasitic agent. The cancer medicament may be selected from the group consisting of a chemotherapeutic agent, an immunotherapeutic agent and a cancer vaccine. The asthma/allergy medicament may be selected from the group consisting of steroids, immunomodulators, anti-inflammatory agents, bronchodilators, leukotriene modifiers, beta₂ agonists, and anti-cholinergics.

In this and other aspects of the invention, the method is a method for treating or preventing a disorder in a subject having or at risk of having the disorder. The disorder may be selected from the group consisting of infectious disease, cancer and asthma or allergy. The subject may be an immunocompromised subject. In other embodiments, the subject is elderly or an infant.

The invention further provides compositions and kits. In one aspect, the invention provides a composition, comprising an imidazoquinoline agent, and an immunostimulatory nucleic acid. In one embodiment, the immunostimulatory nucleic acid is a CpG nucleic acid. In an important embodiment, the immunostimulatory nucleic acid has the nucleotide sequence (#2006) TCG TCG TTT TGT CGT TTT GTC GTT (SEQ ID NO:1).

The invention provides in another aspect another composition comprising an imidazoquinoline agent and an antibody. In one embodiment, the composition further comprises an immunostimulatory nucleic acid.

In still another aspect, the invention provides a composition comprising an imidazoquinoline agent and a disorder-specific medicament. The disorder-specific medicament may be selected from the group consisting of an asthma/allergy medicament, a cancer medicament, and an anti-microbial medicament. In one embodiment, the disorder-specific medicament is an anti-microbial medicament selected from the group consisting of an anti-bacterial agent, an anti-viral agent, an anti-fungal agent, and an anti-parasitic agent. In another embodiment, the disorder-specific medicament is a cancer medicament selected from the group consisting of a chemotherapeutic agent, an immunotherapeutic agent and a cancer vaccine. In still another embodiment, the disorder-specific medicament is an asthma/allergy medicament selected from the group consisting of steroids, immunomodulators, anti-inflammatory agents, bronchodilators, leukotriene modifiers, beta₂ agonists, and anti-cholinergics. One or more species of medicament may be administered to a subject. The composition may further comprise an immunostimulatory nucleic acid.

The compositions may further comprise poly-arginine. In other embodiments, the compositions further comprise an antigen. In still another embodiments, the compositions further comprise an C8-substituted guanosine. In a preferred embodiment, the composition comprises an imidazoquinoline agent, an immunostimulatory nucleic acid, an antigen and poly-arginine. Optionally, the latter composition may also comprise an C8-substituted guanosine.

In another aspect, the invention provides a method for altering the dosage of a therapeutic agent required to prophylactically or therapeutically treat a subject having a disorder (e.g., infectious disease, cancer or asthma/allergy) by co-administering an imidazoquinoline agent with the therapeutic agent. The therapeutic agent may be selected from the group consisting of an antibody, an antigen, an immunostimulatory nucleic acid, an C8-substituted guanosine, and a disorder-specific medicament, but is not so limited. The invention provides a method for increasing the dose of the therapeutic agent that can be administered to a subject in need of such treatment. The method involves administering to a subject in need of such treatment a therapeutic agent in a dose which ordinarily induces side effects and administering to the subject an imidazoquinoline agent in an effective amount to inhibit the side effects. As an example, when the therapeutic agent is a disorder specific medicament such as an anti-cancer therapy (e.g., cancer medicament), common side effects include myelosuppression and microbial infections. Thus, in one embodiment, the side effect is myelosuppression and in another embodiment, the side effect is a microbial infection. In yet another embodiment, the side effect is an adverse allergic reaction.

In another aspect, the invention provides a method for decreasing the dose of a therapeutic agent which can be administered to a subject. The method involves administering to a subject in need of such treatment, a therapeutic agent in a sub-therapeutic dosage and an imidazoquinoline agent, wherein the combination of the sub-therapeutic dose of the therapeutic agent and the imidazoquinoline agent produces a therapeutic result. The method provides several advantages, including lower costs due to the decreased amount of therapeutic agent needed, and a reduced probability of inducing side effects resulting from the therapeutic agent because of the lower doses used.

According to other aspects, the invention involves methods for treating a subject having or at risk of having a disorder by administering an imidazoquinoline agent and a therapeutic agent in different dosing schedules. In one aspect, the invention is a method for treating a subject by administering to a subject in need of such treatment an effective amount of an imidazoquinoline agent, and subsequently administering to the subject a therapeutic agent. In a related aspect, the method involves administering a therapeutic agent to a subject, and subsequently administering an imidazoquinoline agent. In one embodiment, the imidazoquinoline agent is administered on a routine schedule. The routine schedule may be selected from the group consisting of a daily schedule, a weekly schedule, a monthly schedule, a bimonthly schedule, a quarterly schedule, and a semi-annual schedule. In another embodiment, the imidazoquinoline agent is administered on a variable schedule. The imidazoquinoline agent may be administered in a sustained release vehicle.

In other aspects, the invention is a method for treating a subject having a disorder by administering to a subject in need of such treatment a therapeutic agent in an effective amount for providing some symptomatic relief and subsequently administering an imidazoquinoline agent to the subject. In some embodiments, the imidazoquinoline agent is administered in an effective amount for upregulating, enhancing or activating an immune response. In some embodiments, the imidazoquinoline agent is administered in an effective amount for redirecting the immune response a Th1 immune response. In still other embodiments, a plurality of imidazoquinoline agents is administered.

In another aspect, the invention provides a method for treating a subject having or at risk of developing a disorder by administering to a subject in need of such treatment an imidazoquinoline agent and a therapeutic agent, wherein the imidazoquinoline agent is administered systemically and the therapeutic agent is administered locally.

In still another aspect, the invention provides a method for treating a subject having or at risk of developing a disorder by administering to the subject an imidazoquinoline agent on a routine schedule and a therapeutic agent. In other embodiments, the imidazoquinoline agent and/or the therapeutic agent are administered in two or more doses. Alternatively, the imidazoquinoline agent may be administered on a non-regular basis (e.g., at the start of symptoms).

According to another aspect, the invention provides a screening method for comparing Toll-like receptor (TLR) signaling activity of a test compound with TLR signaling activity of an imidazoquinoline. The method involves contacting a functional TLR selected from the group consisting of Toll-like receptor 7 (TLR7) and Toll-like receptor 8 (TLR8) with a reference imidazoquinoline and detecting a reference response mediated by a TLR signal transduction pathway; contacting a functional TLR selected from the group consisting of TLR7 and TLR8 with a test compound and detecting a test response mediated by a TLR signal transduction pathway; and comparing the test response with the reference response to compare the TLR signaling activity of the test compound with the imidazoquinoline. In a preferred embodiment the functional TLR is TLR8. In another preferred embodiment the functional TLR is TLR7.

In certain embodiments the functional TLR is contacted with the reference imidazoquinoline and the test compound independently. In a preferred embodiment the screening method is a method for identifying an imidazoquinoline mimic, wherein when the test response is similar to the reference response the test compound is an imidazoquinoline mimic.

In certain other embodiments the functional TLR is contacted with the reference imidazoquinoline and the test compound concurrently to produce a test-reference response mediated by a TLR signal transduction pathway; the test-reference response may be compared to the reference response. In a preferred embodiment the screening method is a method for identifying an imidazoquinoline agonist, wherein when the test-reference response is greater than the reference response the test compound is an imidazoquinoline agonist. In a preferred embodiment the screening method is a method for identifying an imidazoquinoline antagonist, wherein when the test-reference response is less than the reference response the test compound is an imidazoquinoline antagonist.

In certain embodiments the functional TLR is expressed in a cell. Preferably, the cell is an isolated mammalian cell that naturally expresses functional TLR8. In another preferred embodiment the cell is an isolated mammalian cell that naturally expresses functional TLR7. To facilitate practice of the method, in certain embodiments the cell expressing the functional TLR7 or functional TLR8 includes an expression vector comprising an isolated nucleic acid which encodes a reporter construct selected from the group consisting of interleukin 8 (IL-8), p40 subunit of interleukin 12 (IL-12 p40), nuclear factor kappa B-luciferase (NF-kappa B-luc), p40 subunit of interleukin 12-luciferase (IL-12 p40-luc), and tumor necrosis factor-luciferase (TNF-luc).

In certain other embodiments the functional TLR is part of a cell-free system.

In some embodiments the functional TLR is part of a complex with another TLR, including, for example, TLR1, TLR2, TLR3, TLR4, TLR5, TLR6, TLR7, TLR8, TLR9, or TLR10. The complex can include two or more TLRs.

In certain embodiments the functional TLR is part of a complex with a non-TLR protein selected from the group consisting of myeloid differentiation factor 88 (MyD88), IL-1 receptor-associated kinase (IRAK), tumor necrosis factor receptor-associated factor 6 (TRAF6), I kappa B, NF-kappa B, and functional homologs and derivatives thereof.

In a preferred embodiments the reference imidazoquinoline is R-848 (Resiquimod). In another preferred embodiment the reference imidazoquinoline is R-847 (Imiquimod).

In certain embodiments the test compound is not a nucleic acid molecule. For example, in one embodiment the test compound is a polypeptide. In a preferred embodiment the test compound is an imidazoquinoline other than R-848 or R-847.

In certain embodiments the test compound is a part of a combinatorial library of compounds.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIG. 1 is a bar graph depicting hTLR9-mediated activation of NF-kappa B by CpG ODN 2006, but not by R-848.

FIG. 2A is a bar graph depicting the stimulation index of 293T cells transiently transfected with various hTLR expression vectors in response to exposure to R-848, LPS, control ODN 8954, IL-1, and CpG ODN 2006. Cells were stimulated 24 h after transfection and assayed 16 h later for luciferase activity.

FIG. 2B is a bar graph depicting the R-848 dose-dependent response of 293T cells transiently transfected with various TLR expression constructs.

FIG. 3A is a bar graph depicting response to R-848 of 293-TLR9-Luc cells co-expressing TLR9 and either hTLR7 or hTLR8.

FIG. 3B is a bar graph depicting response of 293-TLR9-LUC cells co-expressing hTLR9 and either hTLR7 or hTLR8 to R-848 and CpG ODN, either individually or together.

FIG. 4 is a bar graph depicting production of IL-8 in 293T cells transiently transfected with different TLR constructs.

FIG. 5A is a bar graph depicting IFN-alpha secretion by human PBMC upon incubation with CpG ODNs or R-848.

FIG. 5B is a graph depicting IFN-alpha secretion by human PBMC following incubation with CpG ODNs and R-848, either individually or together.

FIG. 6A is a bar graph depicting IP-10 secretion by human PBMC upon incubation with CpG ODNs or R-848.

FIG. 6B is a graph depicting IP-10 secretion by human PBMC following incubation with CpG ODNs and R-848, either individually or together.

FIG. 7A is a bar graph depicting TNF-alpha secretion by human PBMC upon incubation with CpG ODNs or R-848.

FIG. 7B is a graph depicting TNF-alpha secretion by human PBMC following incubation with CpG ODNs and R-848, either individually or together.

FIG. 8A is a bar graph depicting IL-10 secretion by human PBMC upon incubation with CpG ODNs or R-848.

FIG. 8B is a graph depicting IL-10 secretion by human PBMC following incubation with CpG ODNs and R-848, either individually or together.

FIG. 9 is a bar graph depicting IL-6 secretion by human PBMC can be partially inhibited by chloroquine.

FIG. 10 is a pair of bar graphs showing (A) the induction of NF-kappa B and (B) the amount of IL-8 produced by 293 fibroblast cells transfected with human TLR9 in response to exposure to various stimuli, including CpG-ODN, GpC-ODN, LPS, and medium.

FIG. 11 is a bar graph showing the induction of NF-kappa B produced by 293 fibroblast cells transfected with murine TLR9 in response to exposure to various stimuli, including CpG-ODN, methylated CpG-ODN (Me-CpG-ODN), GpC-ODN, LPS, and medium.

FIG. 12 is a series of gel images depicting the results of reverse transcriptase-polymerase chain reaction (RT-PCR) assays for murine TLR9 (mTLR9), human TLR9 (hTLR9), and glyceraldehyde-3-phosphate dehydrogenase (GAPDH) in untransfected control 293 cells, 293 cells transfected with mTLR9 (293-mTLR9), and 293 cells transfected with hTLR9 (293-hTLR9).

FIG. 13 is a graph showing the degree of induction of NF-kappa B-luc by various stimuli in stably transfected 293-hTLR9 cells.

FIG. 14 is a graph showing the degree of induction of NF-kappa B-luc by various stimuli in stably transfected 293-mTLR9 cells.

FIG. 15 is a bar graph comparing the ability of CpG nucleic acids and R-848 to augment cytolytic T lymphocyte responses against antigen (e.g., HBsAg) is a mouse model.

FIG. 16 is a graph showing the ability of CpG nucleic acids and R-848 to augment cytolytic T lymphocyte responses against antigen (e.g., HBsAg) is a mouse model as a function of effector to target ratios.

FIG. 17 is a bar graph comparing the ability of CpG nucleic acids and R-848 to augment antibody responses against antigen (e.g., HBsAg) is a mouse model.

FIG. 18 is a bar graph comparing the ability of CpG nucleic acids and R-848 to augment IgG1 and IgG2a antibody responses against antigen (e.g., HBsAg) is a mouse model.

FIG. 19 is a bar graph comparing the ability of CpG nucleic acid, R-848 and Montanide ISA 720 to augment antibody responses against antigen (e.g., HBsAg) is a mouse model.

FIG. 20 is a bar graph comparing the ability of CpG nucleic acid, R-848 and Montanide ISA 720 to augment cytolytic T lymphocyte responses against antigen (e.g., HBsAg) is a mouse model.

It is to be understood that the Figures are not required to enable the invention.

### BRIEF DESCRIPTION OF THE SEQUENCE LISTING

SEQ ID NO:1 is the nucleotide sequence of an immunostimulatory CpG nucleic acid (#2006).

SEQ ID NO:2 is the nucleotide sequence of an immunostimulatory T-rich nucleic acid (#2183).

SEQ ID NO:3 is the nucleotide sequence of a control non-CpG nucleic acid (#1982).

SEQ ID NO:4 is the nucleotide sequence of an immunostimulatory CpG nucleic acid (#8954).

SEQ ID NO:5 is the nucleotide sequence of a negative control nucleic acid (#5177).

SEQ ID NO:6 is the nucleotide sequence of human TLR9 cDNA (GenBank Accession No. AF245704).

SEQ ID NO:7 is the amino acid sequence of human TLR9 protein (GenBank Accession No. AAF78037).

SEQ ID NO:8 is the nucleotide sequence of murine TLR9 cDNA (GenBank Accession No. AF348140).

SEQ ID NO:9 is the amino acid sequence of murine TLR9 protein (GenBank Accession No. AAK29625).

SEQ ID NO:10 is the nucleotide sequence of a control GpC nucleic acid (#2006-GC).

SEQ ID NO:11 is the nucleotide sequence of a methylated CpG nucleic acid (#2006 methylated).

SEQ ID NO:12 is the nucleotide sequence of an immunostimulatory nucleic acid (#1668).

SEQ ID NO:13 is the nucleotide sequence of a GpC nucleic acid (#1668-GC).

SEQ ID NO:14 is the nucleotide sequence of a methylated CpG nucleic acid (#1668 methylated).

SEQ ID NO:15 is the nucleotide sequence of a first primer used to amplify human TLR7 cDNA.

SEQ ID NO:16 is the nucleotide sequence of a second primer used to amplify human TLR7 cDNA.

SEQ ID NO:17 is the nucleotide sequence of human TLR7 cDNA.

SEQ ID NO:18 is the amino acid sequence of human TLR7 protein.

SEQ ID NO:19 is the nucleotide sequence of a first primer used to amplify murine TLR7 cDNA.

SEQ ID NO:20 is the nucleotide sequence of a second primer used to amplify murine TLR7 cDNA.

SEQ ID NO:21 is the nucleotide sequence of murine TLR7 cDNA.

SEQ ID NO:22 is the amino acid sequence of murine TLR7 cDNA.

SEQ ID NO:23 is the nucleotide sequence of a first primer used to amplify human TLR8 cDNA.

SEQ ID NO:24 is the nucleotide sequence of a second primer used to amplify human TLR8 cDNA.

SEQ ID NO:25 is the nucleotide sequence of human TLR8 cDNA.

SEQ ID NO:26 is the amino acid sequence of human TLR8 cDNA.

SEQ ID NO:27 is the amino acid sequence of an N-terminal insertion in human TLR8 corresponding to GenBank Accession No. AF246971.

SEQ ID NO:28 is the nucleotide sequence of a first primer used to amplify murine TLR8 cDNA.

SEQ ID NO:29 is the nucleotide sequence of a second primer used to amplify murine TLR8 cDNA.

SEQ ID NO:30 is the nucleotide sequence of murine TLR8 cDNA.

SEQ ID NO:31 is the amino acid sequence of murine TLR8 protein.

### DETAILED DESCRIPTION OF THE INVENTION

The invention is based, in part, on the surprising discovery that administration of an imidazoquinoline agent and an antibody to a subject enhances antibody-dependent cellular cytoxicity (ADCC). Accordingly, in one aspect, the invention provides methods for treating humans and animals with imidazoquinoline agents in a dose sufficient to induce systemic activation of ADCC. Although not intending to be bound by any particular theory, it is postulated that imidazoquinoline agents enhance systemic ADCC by upregulating expression of Fc receptors and improving the functional activity of effector cells such as monocytes and macrophages. When a therapeutic antibody is co-administered to a subject with an imidazoquinoline agent, the enhanced ADCC activity will lead to a dramatic increase in therapeutic effect.

Imidazoquinolines are immune response modifiers thought to induce expression of several cytokines including interferons (e.g., IFN-alpha and IFN-alpha), TNF-alpha and some interleukins (e.g., IL-1, IL-6 and IL-12). Imidazoquinolines are capable of stimulating a Th1 immune response, as evidenced in part by their ability to induce increases in IgG2a levels. Imidazoquinoline agents reportedly are also capable of inhibiting production of Th2 cytokines such as IL-4, IL-5, and IL-13. Some of the cytokines induced by imidazoquinolines are produced by macrophages and dendritic cells. Some species of imidazoquinolines have been reported to increase NK cell lytic activity and to stimulate B cells proliferation and differentiation, thereby inducing antibody production and secretion.

As used herein, an imidazoquinoline agent includes imidazoquinoline amines, imidazopyridine amines, 6,7-fused cycloalkylimidazopyridine amines, and 1,2 bridged imidazoquinoline amines. These compounds have been described in U.S. Pat. Nos. 4,689,338, 4,929,624, 5,238,944, 5,266,575, 5,268,376, 5,346,905, 5,352,784, 5,389,640, 5,395,937, 5,494,916, 5,482,936, 5,525,612, 6,039,969 and 6,110,929. Particular species of imidazoquinoline agents include R-848 (S-28463); 4-amino-2ethoxymethyl-α,α-dimethyl-1H-imidazo[4,5-c]quinolines-1-ethanol; and 1-(2-methylpropyl)-1H-imidazo[4,5-c]quinolin-4-amine (R-837 or Imiquimod). Imiquimod is currently used in the topical treatment of warts such as genital and anal warts and has also been tested in the topical treatment of basal cell carcinoma.

Antibodies useful in the invention include monoclonal antibodies, polyclonal antibodies, murine antibodies, human antibodies, chimeric murine-human antibodies, and the like. In some embodiments, antibody fragments can be used provided such fragments possess both an Fc and at least one Fab portion.

In some embodiments, the imidazoquinoline is administered at the same time as the antibody, while in other embodiments, it is administered prior to following antibody administration. If delivered prior to the administration of the antibody, the imidazoquinoline agent can be administered 1, 2, 3, 4, 5, 6, 7, or more days prior to the administration of antibody. If administered after the administration of the antibody, the imidazoquinoline agent can be administered 1, 2, 3, 4, 5, 6, 7, or more days after the administration of the antibody. In some preferred embodiments, the imidazoquinoline agent is administered within 48 hours, within 36 hours, within 24 hours, within 12 hours, within 6 hours, or within 4 hours of antibody administration, regardless of whether the antibody is administered prior to or following the imidazoquinoline agent.

Therapeutic antibodies useful in the invention may be specific for microbial antigens (e.g., bacterial, viral, parasitic or fungal antigens), cancer or tumor-associated antigens and self antigens. Preferred antibodies are those that recognize and bind to antigens present on or in a cell. Examples of suitable antibodies include but are not limited to Rituxan™ (rituximab, anti-CD20 antibody), Herceptin (trastuzumab), Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART ID10Ab, SMART ABL 364 Ab, CC49 (mAb B72.3), ImmuRAIT-CEA, anti-IL-4 antibody, an anti-IL-5 antibody, an anti-IL-9 antibody, an anti-Ig antibody, an anti-IgE antibody, serum-derived hepatitis B antibodies, recombinant hepatitis B antibodies, and the like.

Other antibodies similarly useful for the invention include alemtuzumab (B cell chronic lymphocytic leukemia), gemtuzumab ozogamicin (CD33+acute myeloid leukemia), hP67.6 (CD33+acute myeloid leukemia), infliximab (inflammatory bowel disease and rheumatoid arthritis), etanercept (rheumatoid arthritis), tositumomab, MDX-210, oregovomab, anti-EGF receptor mAb, MDX-447, anti-tissue factor protein (TF), (Sunol); ior-c5, c5, edrecolomab, ibritumomab tiuxetan, anti-idiotypic mAb mimic of ganglioside GD3 epitope, anti-HLA-Dr10 mAb, anti-CD33 humanized mAb, anti-CD52 humAb, anti-CD1 mAb (ior t6), MDX-22, celogovab, anti-17-1A mAb, bevacizumab, daclizumab, anti-TAG-72 (MDX-220), anti-idiotypic mAb mimic of high molecular weight proteoglycan (I-Mel-1), anti-idiotypic mAb mimic of high molecular weight proteoglycan (I-Mel-2), anti-CEA Ab, hmAbH11, anti-DNA or DNA-associated proteins (histones) mAb, Gliomab-H mAb, GNI-250 mAb, anti-CD22, CMA 676), anti-idiotypic human mAb to GD2 ganglioside, ior egf/r3, anti-ior c2 glycoprotein mAb, ior c5, anti-FLK-2/FLT-3 mAb, anti-GD-2 bispecific mAb, antinuclear autoantibodies, anti-HLA-DR Ab, anti-CEA mAb, palivizumab, bevacizumab, alemtuzumab, BLyS-mAb, anti-VEGF2, anti-Trail receptor; B3 mAb, mAb BR96, breast cancer; and Abx-Cb1 mAb.

Also included are antibodies such as the following, all of which are commercially available:

Apoptosis Antibodies: BAX Antibodies: Anti-Human Bax Antibodies (Monoclonal),Anti-Human Bax Antibodies (Polyclonal), Anti-Murine Bax Antibodies (Monoclonal), Anti-Murine Bax Antibodies (Polyclonal); Fas/Fas Ligand Antibodies: Anti-Human Fas/Fas Ligand Antibodies, Anti-Murine Fas/Fas Ligand Antibodies Granzyme Antibodies Granzyme B Antibodies; BCL Antibodies: Anti Cytochrome C Antibodies, Anti-Human BCL Antibodies (Monoclonal), Anti-Human bcl Antibodies (Polyclonal), Anti-Murine bcl Antibodies (Monoclonal), Anti-Murine bcl Antibodies (Polyclonal);

Miscellaneous Apoptosis Antibodies: Anti TRADD, TRAIL, TRAFF, DR3 Antibodies Anti-Human Fas/Fas Ligand Antibodies Anti-Murine Fas/Fas Ligand Antibodies;

Miscellaneous Apoptosis Related Antibodies: BIM Antibodies: Anti Human, Murine bim Antibodies (Polyclonal), Anti-Human, Murine bim Antibodies (Monoclonal);

PARP Antibodies Anti-Human PARP Antibodies (Monoclonal) Anti-Human PARP Antibodies(Polyclonal) Anti-Murine PARP Antibodies;

Caspase Antibodies: Anti-Human Caspase Antibodies (Monoclonal), Anti-Murine Caspase Antibodies;

Anti-CD Antibodies: Anti-CD29, PL18-5 PanVera, Anti-CD29, PL4-3 PanVera, Anti-CD41a, PT25-2 PanVera, Anti-CD42b, PL52-4 PanVera, Anti-CD42b, GUR20-5 PanVera, Anti-CD42b, WGA-3 PanVeraAnti-CD43, 1D4 PanVera, Anti-CD46, MCP75-6 PanVera, Anti-CD61, PL11-7 PanVera, Anti-CD61, PL8-5 PanVera, Anti-CD62/P-slctn, PL7-6 PanVera, Anti-CD62/P-slctn, WGA-1 PanVera, Anti-CD154, 5F3 PanVera;

Human Chemokine Antibodies: Human CNTF Antibodies, Human Eotaxin Antibodies, Human Epithelial Neutrophil Activating Peptide-78, Human Exodus Antibodies, Human GRO Antibodies, Human HCC-1 Antibodies, Human I-309 Antibodies, Human IP-10 Antibodies, Human I-TAC Antibodies, Human LIF Antibodies, Human Liver-Expressed Chemokine Antibodies, Human Lymphotaxin Antibodies, Human MCP Antibodies, Human MIP Antibodies, Human Monokine Induced by IFN-gamma Antibodies, Human NAP-2 Antibodies, Human NP-1 Antibodies, Human Platelet Factor-4 Antibodies, Human RANTES Antibodies, Human SDF Antibodies, Human TECK Antibodies;

Murine Chemokine Antibodies: Human B-Cell Attracting Murine Chemokine Antibodies, Chemokine-1 Antibodies, Murine Eotaxin Antibodies, Murine Exodus Antibodies, Murine GCP-2 Antibodies, Murine KC Antibodies, Murine MCP Antibodies, Murine MIP Antibodies, Murine RANTES Antibodies, Rat Chemokine Antibodies, Rat Chemokine Antibodies, Rat CNTF Antibodies, Rat GRO Antibodies, Rat MCP Antibodies, Rat MIP Antibodies, Rat RANTES Antibodies;

Cytokine/Cytokine Receptor Antibodies: Human Biotinylated Cytokine/Cytokine Receptor Antibodies, Human IFN Antibodies, Human IL Antibodies, Human Leptin Antibodies, Human Oncostatin Antibodies, Human TNF Antibodies, Human TNF Receptor Family Antibodies, Murine Biotinylated Cytokine/Cytokine Receptor Antibodies, Murine IFN Antibodies, Murine IL Antibodies, Murine TNF Antibodies, Murine TNF Receptor Antibodies;

Rat Cytokine/Cytokine Receptor Antibodies: Rat Biotinylated Cytokine/Cytokine Receptor Antibodies, Rat IFN Antibodies, Rat IL Antibodies, Rat TNF Antibodies;

ECM Antibodies: Collagen/Procollagen, Laminin, Collagen (Human), Laminin (Human), Procollagen (Human), Vitronectin/Vitronectin Receptor, Vitronectin (Human), Vitronectin Receptor (Human), Fibronectin/Fibronectin Receptor, Fibronectin (Human), Fibronectin Receptor (Human);

Growth Factor Antibodies: Human Growth Factor Antibodies, Murine Growth Factor Antibodies, Porcine Growth Factor Antibodies;

Miscellaneous Antibodies: Baculovirus Antibodies, Cadherin Antibodies, Complement Antibodies, C1q Antibodies, VonWillebrand Factor Antibodies, Cre Antibodies, HIV Antibodies, Influenza Antibodies, Human Leptin Antibodies, Murine Leptin Antibodies, Murine CTLA-4 Antibodies, P450 Antibodies, RNA Polymerase Antibodies;

Neurobio Antibodies: Amyloid Antibodies, GFAP Antibodies, Human NGF Antibodies, Human NT-3 Antibodies, Human NT-4 Antibodies.

Still other antibodies can be used in the invention and these include antibodies listed in references such as the MSRS Catalog of Primary Antibodies, and Linscott's Directory.

The imidazoquinoline agents can also be used with normal and hyper-immune globulin therapy. Normal immune globulin therapy utilizes a antibody product which is prepared from the serum of normal blood donors and pooled. This pooled product contains low titers of antibody to a wide range of antigens such as those of infectious pathogens (e.g., bacteria, viruses such as hepatitis A, parvovirus, enterovirus, fungi and parasites). Hyper-immune globulin therapy utilizes antibodies which are prepared from the serum of individuals who have high titers of an antibody to a particular antigen. Examples of hyper-immune globulins include zoster immune globulin (useful for the prevention of varicella in immunocompromised children and neonates), human rabies immunoglobulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncitial virus infections).

Some commercially available anti-cancer antibodies are listed below along with their commercial source.

Cancer Immunotherapies in Development or on the Market

| MARKETER | BRAND NAME (GENERIC NAME) | INDICATION |
|---|---|---|
| IDEC/Genentech, | Rituxan ™ (rituximab, Mabthera) (IDEC- | non-Hodgkin's lymphoma |
| Inc./Hoffmann-LaRoche (first | C2B8, chimeric murine/human anti-CD20 | |
| monoclonal antibody licensed for | MAb) | |
| the treatment of cancer in the | | |
| U.S.) | | |
| Genentech/Hoffmann-La Roche | Herceptin, anti-Her2 hMAb | Breast/ovarian |
| Cytogen Corp. | Quadramet (CYT-424) radiotherapeutic | Bone metastases |
| | agent | |
| Centocor/Glaxo/Ajinomoto | Panorex ® (17-1A) (murine monoclonal | Adjuvant therapy for |
| | antibody) | colorectal (Dukes-C) |
| Centocor/Ajinomoto | Panorex ® (17-1A) (chimeric murine | Pancreatic, lung, breast, |
| | monoclonal antibody) | ovary |
| IDEC | IDEC-Y2B8 (murine, anti-CD20 MAb | non-Hodgkin's lymhoma |
| | labeled with Yttrium-90) | |
| ImClone Systems | BEC2 (anti-idiotypic MAb, mimics the GD₃ | Small cell lung |
| | epitope) (with BCG) | |
| ImClone Systems | C225 (chimeric monoclonal antibody to | Renal cell |
| | epidermal growth factor receptor (EGFr)) | |
| Techniclone International/Alpha | Oncolym (Lym-1 monoclonal antibody | non-Hodgkin's lymphoma |
| Therapeutics | linked to 131 iodine) | |
| Protein Design Labs | SMART M195 Ab, humanized | Acute myleoid leukemia |
| Techniclone | ¹³¹I LYM-1 (Oncolym ™) | non-Hodgkin's lymphoma |
| Corporation/Cambridge Antibody | | |
| Technology | | |
| Aronex Pharmaceuticals, Inc. | ATRAGEN ® | Acute promyelocytic |
| | | leukemia |
| ImClone Systems | C225 (chimeric anti-EGFr monoclonal | Head & neck, non-small |
| | antibody) + cisplatin or radiation | cell lung cancer |
| Altarex, Canada | Ovarex (B43.13, anti-idiotypic CA125, | Ovarian |
| | mouse MAb) | |
| Coulter Pharma (Clinical results | Bexxar (anti-CD20 Mab labeled with ¹³¹I) | non-Hodgkin's lymphoma |
| have been positive, but the drug | | |
| has been associated with | | |
| significant bone marrow toxicity) | | |
| Aronex Pharmaceuticals, Inc. | ATRAGEN ® | Kaposi's sarcoma |
| IDEC Pharmaceuticals | Rituxan ™ (MAb against CD20) pan-B Ab in | B cell lymphoma |
| Corp./Genentech | combo. with chemotherapy | |
| LeukoSite/Ilex Oncology | LDP-03, huMAb to the leukocyte antigen | Chronic lymphocytic |
| | CAMPATH | leukemia (CLL) |
| Center of Molecular Immunology | ior t6 (anti CD6, murine MAb) CTCL | Cancer |
| Medarex/Novartis | MDX-210 (humanized anti-HER-2 bispecific | Breast, ovarian |
| | antibody) | |
| Medarex/Novartis | MDX-210 (humanized anti-HER-2 bispecific | Prostate, non-small cell |
| | antibody) | lung, pancreatic, breast |
| Medarex | MDX-11 (complement activating receptor | Acute myelogenous |
| | (CAR) monoclonal antibody) | leukemia (AML) |
| Medarex/Novartis | MDX-210 (humanized anti-HER-2 bispecific | Renal and colon |
| | antibody) | |
| Medarex | MDX-11 (complement activating receptor | Ex vivo bone marrow |
| | (CAR) monoclonal antibody) | purging in acute |
| | | myelogenous leukemia |
| | | (AML) |
| Medarex | MDX-22 (humanized bispecific antibody, | Acute myleoid leukemia |
| | MAb-conjugates) (complement cascade | |
| | activators) | |
| Cytogen | OV103 (Yttrium-90 labelled antibody) | Ovarian |
| Cytogen | OV103 (Yttrium-90 labelled antibody) | Prostate |
| Aronex Pharmaceuticals, Inc. | ATRAGEN ® | non-Hodgkin's lymphoma |
| Glaxo Wellcome plc | 3622W94 MAb that binds to EGP40 (17-1A) | non-small cell lung, |
| | pancarcinoma antigen on adenocarcinomas | prostate (adjuvant) |
| Genentech | Anti-VEGF, RhuMAb (inhibits | Lung, breast, prostate, |
| | angiogenesis) | colorectal |
| Protein Design Labs | Zenapax (SMART Anti-Tac (IL-2 receptor) | Leukemia, lymphoma |
| | Ab, humanized) | |
| Protein Design Labs | SMART M195 Ab, humanized | Acute promyelocytic |
| | | leukemia |
| ImClone Systems | C225 (chimeric anti-EGFr monoclonal | Breast |
| | antibody) + taxol | |
| ImClone Systems (licensed from | C225 (chimeric anti-EGFr monoclonal | prostate |
| RPR) | antibody) + doxorubicin | |
| ImClone Systems | C225 (chimeric anti-EGFr monoclonal | prostate |
| | antibody) + adriamycin | |
| ImClone Systems | BEC2 (anti-idiotypic MAb, mimics the GD₃ | Melanoma |
| | epitope) | |
| Medarex | MDX-210 (humanized anti-HER-2 bispecific | Cancer |
| | antibody) | |
| Medarex | MDX-220 (bispecific for tumors that express | Lung, colon, prostate, |
| | TAG-72) | ovarian, endometrial, |
| | | pancreatic and gastric |
| Medarex/Novartis | MDX-210 (humanized anti-HER-2 bispecific | Prostate |
| | antibody) | |
| Medarex/Merck KgaA | MDX-447 (humanized anti-EGF receptor | EGF receptor cancers |
| | bispecific antibody) | (head & neck, prostate, |
| | | lung, bladder, cervical, |
| | | ovarian) |
| Medarex/Novartis | MDX-210 (humanized anti-HER-2 bispecific | Comb. Therapy with G- |
| | antibody) | CSF for various cancers, |
| | | esp. breast |
| IDEC | MELIMMUNE-2 (murine monoclonal | Melanoma |
| | antibody therapeutic vaccine) | |
| IDEC | MELIMMUNE-1 (murine monoclonal | Melanoma |
| | antibody therapeutic vaccine) | |
| Immunomedics, Inc. | CEACIDE ™ (I-131) | Colorectal and other |
| NeoRx | Pretarget ™ radioactive antibodies | non-Hodgkin's B cell |
| | | lymphoma |
| Novopharm Biotech, Inc. | NovoMAb-G2 (pancarcinoma specific Ab) | Cancer |
| Techniclone Corporation/ | TNT (chimeric MAb to histone antigens) | Brain |
| Cambridge Antibody Technology | | |
| Techniclone International/ | TNT (chimeric MAb to histone antigens) | Brain |
| Cambridge Antibody Technology | | |
| Novopharm | Gliomab-H (Monoclonals-Humanized Abs) | Brain, melanomas, |
| | | neuroblastomas |
| Genetics Institute/AHP | GNI-250 Mab | Colorectal |
| Merck KgaA | EMD-72000 (chimeric-EGF antagonist) | Cancer |
| Immunomedics | LymphoCide (humanized LL2 antibody) | non-Hodgkin's B-cell |
| | | lymphoma |
| Immunex/AHP | CMA 676 (monoclonal antibody conjugate) | Acute myelogenous |
| | | leukemia |
| Novopharm Biotech, Inc. | Monopharm-C | Colon, lung, pancreatic |
| Novopharm Biotech, Inc. | 4B5 anti-idiotype Ab | Melanoma, small-cell |
| | | lung |
| Center of Molecular Immunology | ior egf/r3 (anti EGF-R humanized Ab) | Radioimmunotherapy |
| Center of Molecular Immunology | ior c5 (murine MAb colorectal) for | Colorectal |
| | radioimmunotherapy | |
| Creative BioMolecules/ | BABS (biosynthetic antibody binding site) | Breast cancer |
| Chiron | Proteins | |
| ImClone Systems/Chugai | FLK-2 (monoclonal antibody to fetal liver | Tumor-associated |
| | kinase-2 (FLK-2)) | angiogenesis |
| ImmunoGen, Inc. | Humanized MAb/small-drug conjugate | Small-cell lung |
| Medarex, Inc. | MDX-260 bispecific, targets GD-2 | Melanoma, glioma, |
| | | neuroblastoma |
| Procyon Biopharma, Inc. | ANA Ab | Cancer |
| Protein Design Labs | SMART 1D10 Ab | B-cell lymphoma |
| Protein Design Labs/Novartis | SMART ABL 364 Ab | Breast, lung, colon |
| Immunomedics, Inc. | ImmuRAIT-CEA | Colorectal |

The invention is further based, in part, on the surprising discovery that administration of an imidazoquinoline agent and a therapeutic agent has unexpected benefit over the administration of either compound alone. Of particular importance is the use of immunostimulatory nucleic acids, C8-substituted guanosines, antigens, and disorder specific medicaments as therapeutic agents. In one important embodiment, compositions comprising imidazoquinoline agents, immunostimulatory nucleic acids, antigen and a polymer rich in arginine (e.g., poly-arginine), and optionally C8-substituted guanosine are used in the immunomodulatory methods of the invention.

The imidazoquinoline agents are also useful for redirecting an immune response to a Th1 immune response. Redirection of an immune response to a Th1 immune response can be assessed by measuring the levels of cytokines produced in response to the nucleic acid (e.g., by inducing monocytic cells and other cells to produce Th1 cytokines, including IL-12, IFN-alpha and GM-CSF). The redirection or rebalance of the immune response to a Th1 response is particularly useful for the treatment or prevention of asthma. For instance, an effective amount for treating asthma can be that amount useful for redirecting a Th2 type of immune response that is associated with asthma to a Th1 type of response. Th2 cytokines, especially IL-4 and IL-5, are elevated in the airways of asthmatic subjects. These cytokines promote important aspects of the asthmatic inflammatory response, including IgE isotype switching, eosinophil chemotaxis and activation and mast cell growth. Th1 cytokines, especially IFN-alpha and IL-12, can suppress the formation of Th2 clones and production of Th2 cytokines. The imidazoquinoline agents of the invention cause an increase in Th1 cytokines which helps to rebalance the immune system, preventing or reducing the adverse effects associated with a predominately Th2 immune response. The redirection of a Th2 to a Th1 immune response may result in a balanced expression of Th1 and Th2 cytokines or it may result in the induction of more Th1 cytokines than Th2 cytokines.

The invention also includes a method for inducing antigen non-specific innate immune activation and broad spectrum resistance to infectious challenge using the imidazoquinoline agents. The term antigen non-specific innate immune activation as used herein refers to the activation of immune cells other than B cells and for instance can include the activation of NK cells, T cells or other immune cells that can respond in an antigen independent fashion or some combination of these cells. A broad spectrum resistance to infectious challenge is induced because the immune cells are in active form and are primed to respond to any invading compound or microorganism. The cells do not have to be specifically primed against a particular antigen. This is particularly useful in biowarfare, and the other circumstances described above such as travelers.

The stimulation index of a particular imidazoquinoline agent can be tested in various immune cell assays. Preferably, the stimulation index of the imidazoquinoline agent with regard to B cell proliferation is at least about 5, preferably at least about 10, more preferably at least about 15 and most preferably at least about 20 as determined by incorporation of ³H uridine in a murine B cell culture, which has been contacted with 20 μM of nucleic acid for 20 h at 37° C. and has been pulsed with 1 μCi of ³H uridine; and harvested and counted 4 h later as described in detail in U.S. Pat. Nos. 6,207,646B1 and 6,239,116B1 with respect to immunostimulatory nucleic acids. For use in vivo, for example, it is important that the imidazoquinoline agents be capable of effectively inducing an immune response, such as, for example, antibody production.

Currently, some treatment protocols for certain disorders (e.g., cancer) call for the use of IFN-alpha. In one embodiment, the methods of the invention use imidazoquinoline agents as a replacement to the use of alpha-interferon (IFN-alpha) therapy in the treatment of certain disorders. Imidazoquinoline agents can be used to generate IFN-alpha endogenously. In yet other embodiments, the imidazoquinoline agents may be administered along with IFN-alpha. In some embodiments, the targeting agent of the invention or a disorder-specific medicament can also be administered to the subject along with the imidazoquinoline agent and IFN-alpha.

The invention embraces the administration of C8-substituted guanosines either in place of or along with the imidazoquinoline agents in the methods of the invention. C8-substituted guanosines are known to activate both natural killer (NK) cells and macrophages. Guanine ribonucleotides substituted at the C8 position with either a bromine or a thiol group are B cell mitogens and may act as B cell differentiation factors. (Feldbush et al. 1985 J. Immunol. 134:3204; Goodman 1986 J. Immunol. 136:3335.) These compounds have been reported to reduce the IL-2 requirement for NK cell activation. NK and LAK augmenting activities of C8-substituted guanosines appear to be due to their induction of IFN (Thompson, R. A., et al. 1990. cited supra). Examples of C8-substituted guanosines include but are not limited to 8-mercaptoguanosine, 8-bromoguanosine, 8-methylguanosine, 8-oxo-7,8-dihydroguanosine, C8-arylamino-2′-deoxyguanosine, C8-propynyl-guanosine, C8- and N7-substituted guanine ribonucleosides such as 7-allyl-8-oxoguanosine (loxoribine) and 7-methyl-8-oxoguanosine, 8-aminoguanosine, 8-hydroxy-2′-deoxyguanosine, and 8-hydroxyguanosine. 8-mercaptoguanosine and 8-bromoguanosine also can substitute for the cytokine requirement for the generation of MHC restricted CTL (Feldbush 1985. cited supra), augment murine NK activity (Koo et al. 1988. J. Immunol. 140:3249), and synergize with IL-2 in inducing murine LAK generation (Thompson et al. 1990. J. Immunol. 145:3524). In some important embodiments of the invention, C8-substituted guanosines can be used together with or in place of imidazoquinoline agents for the purpose of inducing or enhancing an immune response that includes ADCC.

Certain methods and compositions of the invention comprise the administration or addition of poly-arginine. As used herein, poly-arginine is a homogenous polymer of arginine monomers. Poly-arginine may be of varying length, and may have a peptide backbone but is not so limited. In other embodiments, a polymer rich in arginine can also be used in place of the homogenous polymer of arginine. A polymer rich in arginine can be a polymer that has at least 2 contiguous arginines, at least 3 contiguous arginines, at least 4 contiguous arginines, and at least 5 contiguous arginines, or alternatively it may be a polymer in which at least 20%, at least 30%, at least 40%, at least 50%, at least 60%, at least 70%, at least 80%, or at least 90% of its monomers are arginine residues. It is to be understood, accordingly, that poly-arginine is also a polymer rich in arginine. Because of the positive charge of arginine, polymers rich in arginine (including poly-arginine) serve to neutralize the negative charge associated with some imidazoquinoline agents and the immunostimulatory nucleic acids.

An “immunostimulatory nucleic acid” as used herein is any nucleic acid containing an immunostimulatory motif or backbone that induces an immune response. The immune response may be characterized as, but is not limited to, a Th1-type immune response or a Th2-type immune response. Such immune responses are defined by cytokine and antibody production profiles which are elicited by the activated immune cells. In one preferred embodiment, pan activating immunostimulatory nucleic acids such as #2006 (TCG TCG TTT TGT CGT TTT GTC GTT) are used in combination with the imidazoquinoline agents in the methods of the invention.

Helper (CD4⁺) T cells orchestrate the immune response of mammals through production of soluble factors that act on other immune system cells, including other T cells. Helper CD4⁺, and in some instances also CD8⁺, T cells are characterized as Th1 and Th2 cells (and Tc1 and Tc2 cells if CD8⁺) in both murine and human systems, depending on their cytokine production profiles (Romagnani, 1991, Immunol Today 12: 256-257, Mosmann, 1989, Annu Rev Immunol, 7: 145-173). Th1 cells produce interleukin 2 (IL-2), IL-12, tumor necrosis factor (TNFalpha) and interferon gamma (IFN-gamma) and they are responsible primarily for cell-mediated immunity such as delayed type hypersensitivity. The cytokines that are induced by administration of immunostimulatory nucleic acids are predominantly of the Th1 class. The types of antibodies associated with a Th1 response are generally more protective because they have high neutralization and opsonization capabilities. Th2 cells produce IL-4, IL-5, IL-6, IL-9, IL-10 and IL-13 and are primarily involved in providing optimal help for humoral immune responses such as IgE and IgG4 antibody isotype switching (Mosmann, 1989, Annu Rev Immunol, 7: 145-173). Th2 responses involve predominantly antibodies that have less protective effects against infection.

The terms “nucleic acid” and “oligonucleotide” are used interchangeably to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. Nucleic acid molecules can be obtained from existing nucleic acid sources (e.g., genomic or cDNA), but are preferably synthetic (e.g. produced by nucleic acid synthesis).

Immunostimulatory nucleic acids may possess immunostimulatory motifs such as CpG, poly-G, poly-T, TG, methylated CpG, CpI, and T-rich motifs. In some embodiments of the invention, any nucleic acid, regardless of whether it possesses an identifiable motif, can be used in the combination therapy to modulate an immune response. Immunostimulatory backbones include, but are not limited to, phosphate modified backbones, such as phosphorothioate backbones. Immunostimulatory nucleic acids have been described extensively in the prior art and a brief summary of these nucleic acids is presented below.

In some embodiments, a CpG immunostimulatory nucleic acid is used in the methods of the invention. A CpG immunostimulatory nucleic acid is a nucleic acid which contains a CG dinucleotide, the C residue of which is unmethylated. The effects of CpG nucleic acids on immune modulation have been described extensively in U.S. Patent such as U.S. Pat. No. 6,194,388 B1, U.S. Pat. No. 6,207,646 B1, U.S. Pat. No. 6,239,116 B1 and U.S. Pat. No. 6,218,371 B1, and published patent applications, such as PCT/US98/03678, PCT/US98/10408, PCT/US98/04703, and PCT/US99/09863. The entire contents of each of these patents and patent applications is hereby incorporated by reference.

The terms CpG nucleic acid or CpG oligonucleotide as used herein refer to an immunostimulatory CpG nucleic acid unless otherwise indicated. The entire immunostimulatory nucleic acid can be unmethylated or portions may be unmethylated but at least the C of the 5′ CG 3′ must be unmethylated.

The CpG nucleic acid sequences of the invention include those broadly described above as well as disclosed in issued U.S. Pat. Nos. 6,207,646 B1 and 6,239,116 B1.

In other embodiments of the invention, a non-CpG immunostimulatory nucleic acid is used. A non-CpG immunostimulatory nucleic acid is a nucleic acid which either does not have a CpG motif in its sequence, or has a CpG motif which contains a methylated C residue. In some instances, chimeric oligonucleotides which lack a CpG motif are immunostimulatory and have many of the same prophylactic and therapeutic activities as a CpG oligonucleotide. Non-CpG immunostimulatory nucleic acids may induce Th1 or Th2 immune responses, depending upon their sequence, their mode of delivery and the dose at which they are administered.

Other immunostimulatory nucleic acids that are useful in the invention as targeting agents are Py-rich nucleic acids. Py-rich nucleic acids have similar immune stimulatory properties to CpG oligonucleotides regardless of whether a CpG motif is present. A Py-rich nucleic acid is a T-rich or C-rich immunostimulatory nucleic acid.

An important subset of non-CpG immunostimulatory nucleic acids are T-rich immunostimulatory nucleic acids. The T-rich immunostimulatory nucleic acids of the invention include those disclosed in published PCT patent application PCT/US00/26383, the entire contents of which are incorporated herein by reference. In some embodiments, T-rich nucleic acids 24 bases in length are used. A T-rich nucleic acid is a nucleic acid which includes at least one poly T sequence and/or which has a nucleotide composition of greater than 25% T nucleotide residues. A nucleic acid having a poly-T sequence includes at least four Ts in a row, such as 5′TTTT3′. Preferably the T-rich nucleic acid includes more than one poly T sequence. In preferred embodiments the T-rich nucleic acid may have 2, 3, 4, etc poly T sequences, such as oligonucleotide #2006 (TCG TCG TTT TGT CGT TTT GTC GTT) (SEQ ID NO:1). One of the most highly immunostimulatory T-rich oligonucleotides discovered according to the invention is a nucleic acid composed entirely of T nucleotide residues, e.g., oligonucleotide #2183 (TTT TTT TTT TTT TTT TTT TTT TTT) (SEQ ID NO:2). Other T-rich nucleic acids according to the invention have a nucleotide composition of greater than 25% T nucleotide residues, but do not necessarily include a poly T sequence. In these T-rich nucleic acids the T nucleotide resides may be separated from one another by other types of nucleotide residues, i.e., G, C, and A. In some embodiments, the T-rich nucleic acids have a nucleotide composition of greater than 35%, 40%, 50%, 60%, 70%, 80%, 90%, and 99%, T nucleotide residues and every integer % in between. Preferably the T-rich nucleic acids have at least one poly T sequence and a nucleotide composition of greater than 25% T nucleotide residues.

A C-rich nucleic acid is a nucleic acid molecule having at least one or preferably at least two poly-C regions or which is composed of at least 50% C nucleotides. A poly-C region is at least four C residues in a row. Thus a poly-C region is encompassed by the formula 5′CCCC 3′. In some embodiments it is preferred that the poly-C region have the formula 5′CCCCCC 3′. Other C-rich nucleic acids according to the invention have a nucleotide composition of greater than 50% C nucleotide residues, but do not necessarily include a poly C sequence. In these C-rich nucleic acids the C nucleotide residues may be separated from one another by other types of nucleotide residues, i.e., G, T, and A. In some embodiments the C-rich nucleic acids have a nucleotide composition of greater than 60%, 70%, 80%, 90%, and 99%, C nucleotide residues and every integer % in between. Preferably the C-rich nucleic acids have at least one poly C sequence and a nucleotide composition of greater than 50% C nucleotide residues, and in some embodiments are also T-rich.

TG nucleic acids can also be used in conjunction with the imidazoquinoline agents of the invention for modulating the immune system. Suitable TG nucleic acids are described in published PCT patent application PCT/US00/26383. A “TG nucleic acid” as used herein is a nucleic acid containing at least one TpG dinucleotide (thymidine-guanine dinucleotide sequence, i.e. “TG DNA” or DNA containing a 5′ thymidine followed by 3′ guanosine and linked by a phosphate bond) and activates a component of the immune system.

It has been shown that TG nucleic acids ranging in length from 15 to 25 nucleotides in length can exhibit an increased immune stimulation. Thus, in one aspect, the invention provides an oligonucleotide that is 15-27 nucleotides in length (i.e., an oligonucleotide that is 15, 16, 17, 18, 19, 20, 21, 22, 23, 24, 25, 26 or 27 nucleotides in length) that may be a T-rich nucleic acid or may be a TG nucleic acid, or may be both a T-rich and a TG nucleic acid. Preferably, the TG oligonucleotides range in size from 15 to 25 nucleotides.

Another important subset of non-CpG immunostimulatory nucleic acids are poly-G immunostimulatory nucleic acids. A variety of references, including Pisetsky and Reich, 1993 Mol. Biol. Reports, 18:217-221; Krieger and Herz, 1994, Ann. Rev. Biochem., 63:601-637; Macaya et al., 1993, PNAS, 90:3745-3749; Wyatt et al., 1994, PNAS, 91:1356-1360; Rando and Hogan, 1998, In Applied Antisense Oligonucleotide Technology, ed. Krieg and Stein, p. 335-352; and Kimura et al., 1994, J. Biochem. 116, 991-994 also describe the immunostimulatory properties of poly-G nucleic acids. In accordance with the invention, poly-G-containing nucleotides are useful for treating and preventing bacterial, viral and fungal infections, and can thereby be used to minimize the impact of these infections on the treatment of cancer patients.

Poly-G nucleic acids preferably are nucleic acids having the following formulas:

5′X₁X₂GGGX₃X₄3′

wherein X₁, X₂, X₃, and X₄ are nucleotides. In preferred embodiments at least one of X₃ and X₄ are a G. In other embodiments both of X₃ and X₄ are a G. In yet other embodiments the preferred formula is 5′ GGGNGGG 3′, or 5′ GGGNGGGNGGG 3′ wherein N represents between 0 and 20 nucleotides. In other embodiments the poly G nucleic acid is free of unmethylated CG dinucleotides. In other embodiments the poly G nucleic acid includes at least one unmethylated CG dinucleotide.

The immunostimulatory nucleic acids of the invention can also be those which do not possess CpG, poly-G, or T-rich motifs.

Addition of a poly-A tail to an immunostimulatory nucleic acid can enhance the activity of the nucleic acid. It was discovered that when a highly immunostimulatory CpG nucleic acid (TCG TCG TTT TGT CGT TTT GTC GTT) (SEQ ID NO:1) was modified with the addition of a poly-A tail (AAAAAA) or a poly-T tail (TTTTTT), the resultant oligonucleotides increased in immune stimulatory activity. The ability of the poly-A tail and the poly-T tail to increase the immunostimulating properties of the oligonucleotide was very similar. The highly immunostimulatory CpG nucleic acid described above is a T-rich oligonucleotide. It is likely that if poly-A and poly-T tails are added to a nucleic acid which is not T-rich, it would have a more significant impact on the immunostimulating capability of the nucleic acid. Since the poly-T tail was added to a nucleic acid that was already highly T-rich the immune stimulating properties of the poly-T addition was diluted somewhat, although not completely. This finding has important implications for the use of poly-A regions. Thus in some embodiments the immunostimulatory nucleic acids include a poly-A region and in other embodiments they do not.

Exemplary immunostimulatory nucleic acid sequences include but are not limited to those immunostimulatory sequences described and listed in U.S. Non-Provisional patent application Ser. No. 09/669,187, filed on Sep. 25, 2000, and in corresponding published PCT patent application PCT/US00/26383.

The immunostimulatory nucleic acids can be double-stranded or single-stranded. Generally, double-stranded molecules are more stable in vivo, while single-stranded molecules have increased immune activity. Thus in some aspects of the invention it is preferred that the nucleic acid be single stranded and in other aspects it is preferred that the nucleic acid be double stranded. In certain embodiments, while the nucleic acid is single stranded, it is capable of forming secondary and tertiary structures (e.g., by folding back on itself, or by hybridizing with itself either throughout its entirety or at select segments along its length). Accordingly, while the primary structure of such a nucleic acid may be single stranded, its higher order structures may be double or triple stranded.

For facilitating uptake into cells, the immunostimulatory nucleic acids are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

Nucleic acids having modified backbones, such as phosphorothioate backbones, also fall within the class of immunostimulatory nucleic acids. U.S. Pat. Nos. 5,723,335 and 5,663,153 issued to Hutcherson, et al. and related PCT publication WO95/26204 describe immune stimulation using phosphorothioate oligonucleotide analogues. These patents describe the ability of the phosphorothioate backbone to stimulate an immune response in a non-sequence specific manner.

In the case when the immunostimulatory nucleic acid is administered in conjunction with a nucleic acid vector, such as a vector encoding an antigen, it is preferred that the backbone of the immunostimulatory nucleic acid be a chimeric combination of phosphodiester and phosphorothioate (or other phosphate modification). This is because the uptake of the plasmid vector by the cell may be hindered by the presence of completely phosphorothioate oligonucleotide. Thus when both a vector and an oligonucleotide are delivered to a subject, it is preferred that the oligonucleotide have a chimeric or phosphorothioate and that the plasmid be associated with a vehicle that delivers it directly into the cell, thus avoiding the need for cellular uptake. Such vehicles are known in the art and include, for example, liposomes and gene guns.

The terms nucleic acid and oligonucleotide also encompass nucleic acids or oligonucleotides with substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus modified nucleic acids may include a 2′-O-alkylated ribose group. In addition, modified nucleic acids may include sugars such as arabinose instead of ribose. Thus the nucleic acids may be heterogeneous in backbone composition thereby containing any possible combination of polymer units linked together such as peptide nucleic acids (which have amino acid backbone with nucleic acid bases). In some embodiments, the nucleic acids are homogeneous in backbone composition. Nucleic acids also include substituted purines and pyrimidines such as C-5 propyne modified bases (Wagner et al., Nature Biotechnology 14:840-844, 1996). Purines and pyrimidines include but are not limited to adenine, cytosine, guanine, thymidine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art.

For use in the instant invention, the nucleic acids of the invention can be synthesized de novo using any of a number of procedures well known in the art. For example, the beta-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986,; Garegg et al., Tet. Let. 27:4055-4058, 1986, Gaffney et al., Tet. Let. 29:2619-2622, 1988). These chemistries can be performed by a variety of automated nucleic acid synthesizers available in the market. These nucleic acids are referred to as synthetic nucleic acids. Alternatively, the nucleic acids can be produced on a large scale in plasmids, (see Sambrook, T., et al., “Molecular Cloning: A Laboratory Manual”, Cold Spring Harbor laboratory Press, New York, 1989) and separated into smaller pieces or administered whole. Nucleic acids can be prepared from existing nucleic acid sequences (e.g., genomic or cDNA) using known techniques, such as those employing restriction enzymes, exonucleases or endonucleases. Nucleic acids prepared in this manner are referred to as isolated nucleic acid. An isolated nucleic acid generally refers to a nucleic acid which is separated from components which it is normally associated with in nature. As an example, an isolated nucleic acid may be one which is separated from a cell, from a nucleus, from mitochondria or from chromatin. The term “nucleic acid” encompasses both synthetic and isolated nucleic acid.

For use in vivo, the nucleic acids may optionally be relatively resistant to degradation (e.g., are stabilized). A “stabilized nucleic acid molecule” shall mean a nucleic acid molecule that is relatively resistant to in vivo degradation (e.g., via an exo- or endo-nuclease). Stabilization can be a function of length or secondary structure. Nucleic acids that are tens to hundreds of kbs long are relatively resistant to in vivo degradation. For shorter nucleic acids, secondary structure can stabilize and increase their effect. For example, if the 3′ end of an nucleic acid has self-complementarity to an upstream region, so that it can fold back and form a sort of stem loop structure, then the nucleic acid becomes stabilized and therefore exhibits more activity.

Alternatively, nucleic acid stabilization can be accomplished via phosphate backbone modifications. Preferred stabilized nucleic acids of the instant invention have a modified backbone. It has been demonstrated that modification of the nucleic acid backbone provides enhanced activity of the nucleic acids when administered in vivo. One type of modified backbone is a phosphate backbone modification. Inclusion in immunostimulatory nucleic acids of at least two phosphorothioate linkages at the 5′ end of the oligonucleotide and multiple (preferably five) phosphorothioate linkages at the 3′ end, can in some circumstances provide maximal activity and protect the nucleic acid from degradation by intracellular exo- and endonucleases. Other modified nucleic acids include phosphodiester-modified nucleic acids, combinations of phosphodiester and phosphorothioate nucleic acids, alkylphosphonate and arylphosphonate, alkylphosphorothioate and arylphosphorothioate, methylphosphonate, methylphosphorothioate, phosphorodithioate, p-ethoxy, morpholino, and combinations thereof. Nucleic acids having phosphorothioate linkages provide maximal activity and protect the nucleic acid from degradation by intracellular exo- and endo-nucleases. and combinations thereof. Each of these combinations and their particular effects on immune cells is discussed in more detail with respect to CpG nucleic acids in issued U.S. Pat. Nos. 6,207,646 B1 and 6,239,116 B1, the entire contents of which are hereby incorporated by reference. It is believed that these modified nucleic acids may show more stimulatory activity due to enhanced nuclease resistance, increased cellular uptake, increased protein binding, and/or altered intracellular localization.

The compositions of the invention may optionally be chimeric oligonucleotides. The chimeric oligonucleotides are oligonucleotides having a formula: 5′ Y₁N₁ZN₂Y₂3′. Y₁ and Y₂ are nucleic acid molecules having between 1 and 10 nucleotides. Y₁ and Y₂ each include at least one modified internucleotide linkage. Since at least 2 nucleotides of the chimeric oligonucleotides include backbone modifications these nucleic acids are an example of one type of “stabilized immunostimulatory nucleic acids.” With respect to the chimeric oligonucleotides, Y₁ and Y₂ are considered independent of one another. This means that each of Y₁ and Y₂ may or may not have different sequences and different backbone linkages from one anther in the same molecule. The sequences vary, but in some cases Y₁ and Y₂ have a poly-G sequence. A poly-G sequence refers to at least 3 Gs in a row. In other embodiments the poly-G sequence refers to at least 4, 5, 6, 7, or 8 Gs in a row. In other embodiments Y₁ and Y₂ may be TCGTCG, TCGTCGT, or TCGTCGTT. Y₁ and Y₂ may also have a poly-C, poly-T, or poly-A sequence. In some embodiments Y₁ and/or Y₂ have between 3 and 8 nucleotides. N₁ and N₂ are nucleic acid molecules having between 0 and 5 nucleotides as long as N₁ZN₂ has at least 6 nucleotides in total. The nucleotides of N₁ZN₂ have a phosphodiester backbone and do not include nucleic acids having a modified backbone. Z is an immunostimulatory nucleic acid motif but does not include a CG. For instance, Z may be a nucleic acid a T-rich sequence, e.g. including a TTTT motif or a sequence wherein at least 50% of the bases of the sequence are Ts or Z may be a TG sequence.

The center nucleotides (N₁ZN₂) of the formula Y₁N₁ZN₂Y₂ have phosphodiester internucleotide linkages and Y₁ and Y₂ have at least one, but may have more than one or even may have all modified internucleotide linkages. In preferred embodiments Y₁ and/or Y₂ have at least two or between two and five modified internucleotide linkages or Y₁ has two modified internucleotide linkages and Y₂ has five modified internucleotide linkages or Y₁ has five modified internucleotide linkages and Y₂ has two modified internucleotide linkages. The modified internucleotide linkage, in some embodiments is a phosphorothioate modified linkage, a phosphorodithioate modified linkage or a p-ethoxy modified linkage.

Modified backbones such as phosphorothioates may be synthesized using automated techniques employing either phosphoramidate or H-phosphonate chemistries. Aryl-and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

Other stabilized nucleic acids include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

Both phosphorothioate and phosphodiester nucleic acids containing immunostimulatory motifs are active in immune cells. However, based on the concentration needed to induce immunostimulatory nucleic acid specific effects, the nuclease resistant phosphorothioate backbone immunostimulatory nucleic acids are more potent than phosphodiester backbone immunostimulatory nucleic acids. For example, 2 μg/ml of the phosphorothioate has been shown to effect the same immune stimulation as a 90 μg/ml of the phosphodiester.

Another type of modified backbone, useful according to the invention, is a peptide nucleic acid. The backbone is composed of aminoethylglycine and supports bases which provide the DNA character. The backbone does not include any phosphate and thus may optionally have no net charge. The lack of charge allows for stronger DNA-DNA binding because the charge repulsion between the two strands does not exist. Additionally, because the backbone has an extra methylene group, the oligonucleotides are enzyme/protease resistant. Peptide nucleic acids can be purchased from various commercial sources, e.g., Perkin Elmer, or synthesized de novo.

Another class of backbone modifications include 2′-O-methylribonucleosides (2′-Ome). These types of substitutions are described extensively in the prior art and in particular with respect to their immunostimulating properties in Zhao et al., Bioorganic and Medicinal Chemistry Letters, 1999, 9:24:3453. Zhao et al. describes methods of preparing 2′-Ome modifications to nucleic acids.

The nucleic acid molecules of the invention may include naturally-occurring or synthetic purine or pyrimidine heterocyclic bases as well as modified backbones. Purine or pyrimidine heterocyclic bases include, but are not limited to, adenine, guanine, cytosine, thymidine, uracil, and inosine. Other representative heterocyclic bases are disclosed in U.S. Pat. No. 3,687,808, issued to Merigan, et al. The terms “purines” or “pyrimidines” or “bases” are used herein to refer to both naturally-occurring or synthetic purines, pyrimidines or bases.

The immunostimulatory nucleic acids having backbone modifications useful according to the invention in some embodiments are S- or R-chiral immunostimulatory nucleic acids. An “S chiral immunostimulatory nucleic acid” as used herein is an immunostimulatory nucleic acid wherein at least two nucleotides have a backbone modification forming a chiral center and wherein a plurality of the chiral centers have S chirality. An “R chiral immunostimulatory nucleic acid” as used herein is an immunostimulatory nucleic acid wherein at least two nucleotides have a backbone modification forming a chiral center and wherein a plurality of the chiral centers have R chirality. The backbone modification may be any type of modification that forms a chiral center. The modifications include but are not limited to phosphorothioate, methylphosphonate, methylphosphorothioate, phosphorodithioate, 2′-Ome and combinations thereof.

The chiral immunostimulatory nucleic acids must have at least two nucleotides within the nucleic acid that have a backbone modification. All or less than all of the nucleotides in the nucleic acid, however, may have a modified backbone. Of the nucleotides having a modified backbone (referred to as chiral centers), a plurality have a single chirality, S or R. A “plurality” as used herein refers to an amount greater than 75%. Thus, less than all of the chiral centers may have S or R chirality as long as a plurality of the chiral centers have S or R chirality. In some embodiments at least 80,%, 85%, 90%, 95%, or 100% of the chiral centers have S or R chirality. In other embodiments at least 80%, 85%, 90%, 95%, or 100% of the nucleotides have backbone modifications.

The S- and R- chiral immunostimulatory nucleic acids may be prepared by any method known in the art for producing chirally pure oligonucleotides. Stec et al teach methods for producing stereopure phosphorothioate oligodeoxynucleotides using an oxathiaphospholane. Stec W J et al. (1995) J Am Chem Soc 117:12019. Other methods for making chirally pure oligonucleotides have been described by companies such as ISIS Pharmaceuticals. U.S. patents which disclose methods for generating stereopure oligonucleotides include U.S. Pat. Nos. 5,883,237, 5,837,856, 5,599,797, 5,512,668, 5,856,465, 5,359,052, 5,506,212, 5,521,302 and 5,212,295, each of which is hereby incorporated by reference in its entirety.

One or more immunostimulatory nucleic acids which may or may not differ in terms of their profile, sequence, backbone modifications and biological effect may be administered to a subject. As an example, CpG nucleic acids and T-rich nucleic acids may be administered to a single subject along with an imidazoquinoline agent. In another example, a plurality of CpG nucleic acids which differ in nucleotide sequence may also be administered to a subject along with the imidazoquinoline agent.

The immunostimulatory nucleic acids may be delivered to the subject in the form of a plasmid vector. In some embodiments, one plasmid vector could include both the immunostimulatory nucleic acid and a nucleic acid encoding a disorder-specific medicament and/or an antigen if either can be encoded by a nucleic acid. In still other embodiments, the plasmid may encode proteins or polypeptides involved in the stimulation or regulation of an immune response such as IFN-alpha, CD80, and the like. The immunostimulatory nucleic acid may be present in the coding sequences of the plasmid, however, their location is not so limited. In other embodiments, separate plasmids could be used. In yet other embodiments, no plasmids could be used.

The therapeutic agents described herein including imidazoquinoline agents, antigens, immunostimulatory nucleic acids, antibodies, C8-substituted guanosines, as well as the polymers rich in arginine can be physically combined without the need for covalent bonding between their substituents when used in the methods of the invention. Alternatively, they may also be conjugated in various combinations either directly or indirectly using linking molecules, as described below.

Examples of suitable linking molecules which can be used include bifunctional crosslinker molecules. The bifunctional crosslinker molecules may be homobifunctional or heterobifunctional, depending upon the nature of the molecules to be conjugated. Homobifunctional crosslinkers have two identical reactive groups. Heterobifunctional crosslinkers are defined as having two different reactive groups that allow for sequential conjugation reaction. Various types of commercially available crosslinkers are reactive with one or more of the following groups: primary amines, secondary amines, sulphydryls, carboxyls, carbonyls and carbohydrates. Examples of amine-specific crosslinkers are bis(sulfosuccinimidyl) suberate, bis[2-(succinimidooxycarbonyloxy)ethyl]sulfone, disuccinimidyl suberate, disuccinimidyl tartarate, dimethyl adipimate.2 HCl, dimethyl pimelimidate.2 HCl, dimethyl suberimidate.2 HCl, and ethylene glycolbis-[succinimidyl-[succinate]]. Crosslinkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido) butyl]-3′-[2′-pyridyldithio]propionamide. Crosslinkers preferentially reactive with carbohydrates include azidobenzoyl hydrazine. Crosslinkers preferentially reactive with carboxyl groups include 4-[p-azidosalicylamido]butylamine. Heterobifunctional crosslinkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional crosslinkers that react with carboxyl and amine groups include 1-ethyl-3-[[3-dimethylaminopropyl]carbodiimide hydrochloride. Heterobifunctional crosslinkers that react with carbohydrates and sulfhydryls include 4-[N-maleimidomethyl]-cyclohexane-1-carboxylhydrazide.2 HCl, 4-(4-N-maleimidophenyl)-butyric acid hydrazide.2 HCl, and 3-[2-pyridyldithio]propionyl hydrazide. The crosslinkers are bis-[beta-4-azidosalicylamido)ethyl]disulfide and glutaraldehyde. Amine or thiol groups may be added at any nucleotide of a synthetic nucleic acid molecule so as to provide a point of attachment for a bifunctional crosslinker molecule. The nucleic acid molecule may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

The imidazoquinoline agents together with the other agents described herein are useful in some aspects of the invention in the prophylaxis and treatment of subjects having or at risk of developing (i.e., at risk of having) a disorder. Generally, the disorders to be prevented and/or treated by the methods provided herein are those that would benefit from a stimulated immune response. In important embodiments, the disorders targeted by the methods and compositions of the invention include cancer, infectious disease, and asthma and allergy. The disorder may also be warts.

The invention intends to treat subjects who are at risk of developing particular disorders (e.g., infectious disease, cancer, asthma, allergy and disorders characterized by warts), as well as subjects that have such disorders. As used herein, the term treat, treated, or treating when used with respect to one of the disorders described herein refers to a prophylactic treatment which decreases the likelihood that the subject will develop the disorder as well as a treatment after the subject has developed the disorder, e.g., reduce or eliminate the disorder or prevent it from becoming worse. Subjects at risk are defined as those who have a higher than normal risk of developing the disorder. The normal risk is generally the risk of a population of normal individuals who do not have the disorder and are not at risk of developing it.

Thus, in prophylactic methods of the invention, the subjects to be treated include those that are at risk of developing an infectious disease, those at risk of developing cancer, and those at risk of developing asthma or allergy. A subject at risk of developing a disorder generally refers to a subject that has a greater likelihood of having the disorder than the population on average.

A subject shall mean a human or animal including but not limited to a dog, cat, horse, cow, pig, sheep, goat, chicken, rodent e.g., rats and mice, primate, e.g., monkey, and fish or aquaculture species such as fin fish (e.g., salmon) and shellfish (e.g., shrimp and scallops). Subjects suitable for therapeutic or prophylactic methods include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. Although many of the embodiments described herein relate to human disorders, the invention is also useful for treating other nonhuman vertebrates. Nonhuman vertebrates are also capable of being treated with the imidazoquinoline agents disclosed herein.

An “infectious disease” as used herein, refers to a disorder arising from the invasion of a host, superficially, locally, or systemically, by an infectious organism. Infectious organisms include bacteria, viruses, fungi, and parasites. Accordingly, “infectious disease” includes bacterial infections, viral infections, fungal infections and parasitic infections.

Bacteria are unicellular organisms which multiply asexually by binary fission. They are classified and named based on their morphology, staining reactions, nutrition and metabolic requirements, antigenic structure, chemical composition, and genetic homology. Bacteria can be classified into three groups based on their morphological forms, spherical (coccus), straight-rod (bacillus) and curved or spiral rod (vibrio, campylobacter, spirillum, and spirochaete). Bacteria are also more commonly characterized based on their staining reactions into two classes of organisms, gram-positive and gram-negative. Gram refers to the method of staining which is commonly performed in microbiology labs. Gram-positive organisms retain the stain following the staining procedure and appear a deep violet color. Gram-negative organisms do not retain the stain but take up the counter-stain and thus appear pink. U.S. Non-Provisional patent application Ser. No. 09/801,839, filed Mar. 8, 2001, lists a number of bacteria, the infections of which the present invention intends to prevent and treat.

Viruses are small infectious agents which generally contain a nucleic acid core and a protein coat, but are not independently living organisms. Viruses can also take the form of infectious nucleic acids lacking a protein. A virus cannot survive in the absence of a living cell within which it can replicate. Viruses enter specific living cells either by endocytosis or direct injection of DNA (phage) and multiply, causing disease. The multiplied virus can then be released and infect additional cells. Some viruses are DNA-containing viruses and other are RNA-containing viruses.

Viruses include, but are not limited to, interoviruses (including, but not limited to, viruses that the family picornaviridae, such as polio virus, coxsackie virus, echo virus), rotaviruses, adenovirus, hepatitis.

Infectious viruses of both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses.

U.S. Non-Provisional patent application Ser. No. 09/801,839, filed Mar. 8, 2001, lists a number of viruses, the infections of which the present invention intends to prevent and treat.

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as amatatoxin and phallotoxin produced by poisonous mushrooms and aflotoxins, produced by aspergillus species, not all fungi cause infectious disease.

Infectious fungi can cause systemic or superficial infections. Primary systemic infection can occur in normal healthy subjects and opportunistic infections, are most frequently found in immuno-compromised subjects. The most common fungal agents causing primary systemic infection include blastomyces, coccidioides, and histoplasma. Common fungi causing opportunistic infection in immuno-compromised or immunosuppressed subjects include, but are not limited to, candida albicans, cryptococcus neoformans, and various aspergillus species. Systemic fungal infections are invasive infections of the internal organs. The organism usually enters the body through the lungs, gastrointestinal tract, or intravenous lines. These types of infections can be caused by primary pathogenic fungi or opportunistic fungi.

Superficial fungal infections involve growth of fungi on an external surface without invasion of internal tissues. Typical superficial fungal infections include cutaneous fungal infections involving skin, hair, or nails.

Diseases associated with fungal infection include aspergillosis, blastomycosis, camdidiais, chromoblastomycosis, coccidioidomycosis, cryptococcosis, fungal eye infections, fungal hair, nail, and skin infections, histoplasmosis, lobomycosis, mycetoma, otomycosis, paracoccidioidomycosis, penicilliosis, marneffeii, phaeohyphomycosis, rhinosporidioisis, sporotrichosis, and zygomycosis.

U.S. Non-Provisional patent application Ser. No. 09/306,281, filed Mar. 8, 2001, lists a number of fungi, the infections of which the present invention intends to prevent and treat.

Parasites are organisms which depend upon other organisms in order to survive and thus must enter, or infect, another organism to continue their life cycle. The infected organism, i.e., the host, provides both nutrition and habitat to the parasite. Although in its broadest sense the term parasite can include all infectious agents (i.e., bacteria, viruses, fungi, protozoa and helminths), generally speaking, the term is used to refer solely to protozoa, helminths, and ectoparasitic arthropods (e.g., ticks, mites, etc.). Protozoa are single celled organisms which can replicate both intracellularly and extracellularly, particularly in the blood, intestinal tract or the extracellular matrix of tissues. Helminths are multicellular organisms which almost always are extracellular (the exception being Trichinella spp.). Helminths normally require exit from a primary host and transmission into a secondary host in order to replicate. In contrast to these aforementioned classes, ectoparasitic arthropods form a parasitic relationship with the external surface of the host body.

Parasites include intracellular parasites and obligate intracellular parasites. Examples of parasites include but are not limited to Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis and Leishmania tropica, Trypanosoma gambiense, Trypanosmoma rhodesiense and Schistosoma mansoni.

U.S. Non-Provisional patent application Ser. No. 09/306,281, filed May 6, 1999, lists a number of other parasites, the infections of which the present invention intends to prevent and treat.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative, and is not intended to be limiting.

In some aspects, the invention also intends to treat diseases in which prions are implicated in disease progression such as for example bovine spongiform encephalopathy (i.e., mad cow disease) or scrapie infection in animals, or Creutzfeldt-Jakob disease in humans.

In some important embodiments, the methods of the invention are intended to treat or prevent infection ssuch as small pox or anthrax infections.

A subject having an infectious disease is a subject that has been exposed to an infectious organism and has acute or chronic detectable levels of the organism in the body. Exposure to the infectious organism generally occurs with the external surface of the subject, e.g., skin or mucosal membranes and/or refers to the penetration of the external surface of the subject by the infectious organism.

A subject at risk of developing an infectious disease is a subject who has a higher than normal risk of exposure to an infection causing pathogen. For instance, a subject at risk may be a subject who is planning to travel to an area where a particular type of infectious agent is found or it may be a subject who through lifestyle or medical procedures is exposed to bodily fluids which may contain infectious organisms or directly to the organism or a subject living in an area where an infectious organism has been identified. Subjects at risk of developing an infectious disease also include general populations to which a medical agency recommends vaccination against a particular infectious organism.

A subject at risk of developing an infectious disease includes those subjects that have a general risk of exposure to a microorganism, e.g., influenza, but that don't have the active disease during the treatment of the invention as well as subjects that are considered to be at specific risk of developing an infectious disease because of medical or environmental factors, that expose them to a particular microorganism.

Cancer is a disease which involves the uncontrolled growth (i.e., division) of cells. Some of the known mechanisms which contribute to the uncontrolled proliferation of cancer cells include growth factor independence, failure to detect genomic mutation, and inappropriate cell signaling. The ability of cancer cells to ignore normal growth controls may result in an increased rate of proliferation. Although the causes of cancer have not been firmly established, there are some factors known to contribute, or at least predispose a subject, to cancer. Such factors include particular genetic mutations (e.g., BRCA gene mutation for breast cancer, APC for colon cancer), exposure to suspected cancer-causing agents, or carcinogens (e.g., asbestos, UV radiation) and familial disposition for particular cancers such as breast cancer.

The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

A subject having a cancer is a subject that has detectable cancerous cells.

A subject at risk of developing a cancer is one who has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

An “allergy” refers to acquired hypersensitivity to a substance (allergen). Allergic conditions include but are not limited to eczema, allergic rhinitis or coryza, hay fever, conjunctivitis, bronchial asthma, urticaria (hives) and food allergies, and other atopic conditions atopic dermatitis; anaphylaxis; drug allergy; angioedema; and allergic conjunctivitis. Allergic diseases in dogs include but are not limited to seasonal dermatitis; perennial dermatitis; rhinitis: conjunctivitis; allergic asthma; and drug reactions. Allergic diseases in cats include but are not limited to dermatitis and respiratory disorders; and food allergens. Allergic diseases in horses include but are not limited to respiratory disorders such as “heaves” and dermatitis. Allergic diseases in non-human primates include but are not limited to allergic asthma and allergic dermatitis.

Allergy is a disease associated with the production of antibodies from a particular class of immunoglobulin, IgE, against allergens. The development of an IgE-mediated response to common aeroallergens is also a factor which indicates predisposition towards the development of asthma. If an allergen encounters a specific IgE which is bound to an Fc IgE receptor on the surface of a basophil (circulating in the blood) or mast cell (dispersed throughout solid tissue), the cell becomes activated, resulting in the production and release of mediators such as histamine, scrotonin, and lipid mediators. Allergic diseases include but are not limited to rhinitis (hay fever) asthma, urticaria and atopic dermatitis.

A subject having an allergy is a subject that is currently experiencing or has previously experienced an allergic reaction in response to an allergen.

A subject at risk of developing an allergy or asthma is a subject that has been identified as having an allergy or asthma in the past but who is not currently experiencing the active disease as well as a subject that is considered to be at risk of developing asthma or allergy because of genetic or environmental factors. A subject at risk of developing allergy or asthma can also include a subject who has any risk of exposure to an allergen or a risk of developing asthma, i.e. someone who has suffered from an asthmatic attack previously or has a predisposition to asthmatic attacks. For instance, a subject at risk may be a subject who is planning to travel to an area where a particular type of allergen or asthmatic initiator is found or it may even be any subject living in an area where an allergen has been identified. If the subject develops allergic responses to a particular antigen and the subject may be exposed to the antigen, i.e., during pollen season, then that subject is at risk of exposure to the antigen.

Currently, allergic diseases are generally treated by the injection of small doses of antigen followed by subsequent increasing dosage of antigen. It is believed that this procedure induces tolerization to the allergen to prevent further allergic reactions. These methods, however, can take several years to be effective and are associated with the risk of side effects such as anaphylactic shock. The methods of the invention avoid these problems.

Allergies are generally caused by IgE antibody generation against harmless allergens. The cytokines that are induced by systemic or mucosal administration of imidazoquinoline agents are predominantly of a class called Th1 (examples are IL-12, IFN-alpha and IFN-gamma) and these induce both humoral and cellular immune responses. The types of antibodies associated with a Th1 response are generally more protective because they have high neutralization and opsonization capabilities. The other major type of immune response, which is associated with the production of IL-4, IL-5 and IL-10 cytokines, is termed a Th2 immune response. Th2 responses involve predominately antibodies and these have less protective effect against infection and some Th2 isotypes (e.g., IgE) are associated with allergy. In general, it appears that allergic diseases are mediated by Th2 type immune responses while Th1 responses provide the best protection against infection, although excessive Th1 responses are associated with autoimmune disease. Based on the ability of the imidazoquinoline agents to shift the immune response in a subject to a Th1 response (which is protective against allergic reactions), an effective dose for inducing an immune response of a imidazoquinoline agent can be administered to a subject to treat or prevent an allergy.

The generic name for molecules that cause an allergic reaction is allergen. There are numerous species of allergens. The allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in man. Histamine and the other vasoactive amines are normally stored in mast cells and basophil leukocytes. The mast cells are dispersed throughout animal tissue and the basophils circulate within the vascular system. These cells manufacture and store histamine within the cell unless the specialized sequence of events involving IgE binding occurs to trigger its release.

The symptoms of the allergic reaction vary, depending on the location within the body where the IgE reacts with the antigen. If the reaction occurs along the respiratory epithelium the symptoms are sneezing, coughing and asthmatic reactions. If the interaction occurs in the digestive tract, as in the case of food allergies, abdominal pain and diarrhea are common. Systematic reactions, for example following a bee sting, can be severe and often life threatening.

Delayed type hypersensitivity, also known as type IV allergy reaction is an allergic reaction characterized by a delay period of at least 12 hours from invasion of the antigen into the allergic subject until appearance of the inflammatory or immune reaction. The T lymphocytes (sensitized T lymphocytes) of individuals in an allergic condition react with the antigen, triggering the T lymphocytes to release lymphokines (macrophage migration inhibitory factor (MIF), macrophage activating factor (MAF), mitogenic factor (MF), skin-reactive factor (SRF), chemotactic factor, neovascularization-accelerating factor, etc.), which function as inflammation mediators, and the biological activity of these lymphokines, together with the direct and indirect effects of locally appearing lymphocytes and other inflammatory immune cells, give rise to the type IV allergy reaction. Delayed allergy reactions include tuberculin type reaction, homograft rejection reaction, cell-dependent type protective reaction, contact dermatitis hypersensitivity reaction, and the like, which are known to be most strongly suppressed by steroidal agents. Consequently, steroidal agents are effective against diseases which are caused by delayed allergy reactions. Long-term use of steroidal agents at concentrations currently being used can, however, lead to the serious side-effect known as steroid dependence. The methods of the invention solve some of these problems, by providing for lower and fewer doses to be administered.

Immediate hypersensitivity (or anaphylactic response) is a form of allergic reaction which develops very quickly, i.e. within seconds or minutes of exposure of the patient to the causative allergen, and it is mediated by IgE antibodies made by B lymphocytes. In nonallergic patients, there is no IgE antibody of clinical relevance; but, in a person suffering with allergic diseases, IgE antibody mediates immediate hypersensitivity by sensitizing mast cells which are abundant in the skin, lymphoid organs, in the membranes of the eye, nose and mouth, and in the respiratory tract and intestines.

Mast cells have surface receptors for IgE, and the IgE antibodies in allergy-suffering patients become bound to them. As discussed briefly above, when the bound IgE is subsequently contacted by the appropriate allergen, the mast cell is caused to degranulate and to release various substances called bioactive mediators, such as histamine, into the surrounding tissue. It is the biologic activity of these substances which is responsible for the clinical symptoms typical of immediate hypersensitivity; namely, contraction of smooth muscle in the airways or the intestine, the dilation of small blood vessels and the increase in their permeability to water and plasma proteins, the secretion of thick sticky mucus, and in the skin, redness, swelling and the stimulation of nerve endings that results in itching or pain.

The imidazoquinoline agents have significant therapeutic utility in the treatment of allergic and non-allergic conditions such as asthma, particularly when used in combination with other therapeutic agents (e.g., those used to regulate levels of proinflammatory cytokines). Th2 cytokines, especially IL-4 and IL-5 are elevated in the airways of asthmatic subjects. These cytokines promote important aspects of the asthmatic inflammatory response, including IgE isotope switching, eosinophil chemotaxis and activation and mast cell growth. Th1 cytokines, especially IFN-gamma and IL-12, can suppress the formation of Th2 clones and production of Th2 cytokines. Asthma refers to a disorder of the respiratory system characterized by inflammation, narrowing of the airways and increased reactivity of the airways to inhaled agents. Asthma is frequently, although not exclusively associated with atopic or allergic symptoms. In some of the preceding aspects of the invention related to asthma and allergy, the imidazoquinoline agents of the invention are not administered directly to the lungs of the subject.

Symptoms of asthma include recurrent episodes of wheezing, breathlessness, and chest tightness, and coughing, resulting from airflow obstruction. Airway inflammation associated with asthma can be detected through observation of a number of physiological changes, such as, denudation of airway epithelium, collagen deposition beneath basement membrane, edema, mast cell activation, inflammatory cell infiltration, including neutrophils, eosinophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, subbasement membrane fibrosis may occur, leading to persistent abnormalities in lung function.

Research over the past several years has revealed that asthma likely results from complex interactions among inflammatory cells, mediators, and other cells and tissues resident in the airway. Mast cells, eosinophils, epithelial cells, macrophage, and activated T-cells all play an important role in the inflammatory process associated with asthma (Djukanovic et al., Am. Rev. Respir. Dis; 142:434-457; 1990). It is believed that these cells can influence airway function through secretion of preformed and newly synthesized mediators which can act directly or indirectly on the local tissue. It has also been recognized that subpopulations of T-lymphocytes (Th2) play an important role in regulating allergic inflammation in the airway by releasing selective cytokines and establishing disease chronicity (Robinson, et al. N. Engl. J. Med.; 326:298-304; 1992).

Asthma is a complex disorder which arises at different stages in development and can be classified based on the degree of symptoms of acute, subacute or chronic. An acute inflammatory response is associated with an early recruitment of cells into the airway. The subacute inflammatory response involves the recruitment of cells as well as the activation of resident cells causing a more persistent pattern of inflammation. Chronic inflammatory response is characterized by a persistent level of cell damage and an ongoing repair process, which may result in permanent abnormalities in the airway.

A “subject having asthma” is a subject that has a disorder of the respiratory system characterized by inflammation, narrowing of the airways and increased reactivity of the airways to inhaled agents. Asthma is frequently, although not exclusively associated with atopic or allergic symptoms. An “initiator” as used herein refers to a composition or environmental condition which triggers asthma. Initiators include, but are not limited to, allergens, cold temperatures, exercise, viral infections, SO₂.

In another aspect the invention provides methods for treating or preventing a disorder in a hypo-responsive subject. As used herein, a hypo-responsive subject is one who has previously failed to respond to a treatment directed at treating or preventing the disorder or one who is at risk of not responding to such a treatment.

Other subjects who are hypo-responsive include those who are refractory to a disorder-specific medicament. As used herein, the term “refractory” means resistant or failure to yield to treatment. Such subjects may be those who never responded to the medicament (i.e., subjects who are non-responders), or alternatively, they may be those who at one time responded to the medicament, but have since that time have become refractory to it. In some embodiments, the subject is one who is refractory to a subset of medicaments. A subset of medicaments is at least one medicament. In some embodiments, a subset refers to 2, 3, 4, 5, 6, 7, 8, 9, or 10 medicaments.

In other embodiments, hypo-responsive subjects are elderly subjects, regardless of whether they have or have not previously responded to a treatment directed at treating or preventing the disorder. Elderly subjects, even those who have previously responded to such treatment, are considered to be at risk of not responding to a future administration of this treatment. Similarly, neonatal subjects are also considered to be at risk of not responding to treatment directed at treating or preventing the disorder. In important embodiments, the disorder is asthma or allergy.

In some aspects, the methods of the invention include exposing the subject to be treated with an antigen prior to, concurrently with, or subsequent to the administration of an imidazoquinoline agent.

As used herein, the term “exposed to” refers to either the active step of contacting the subject with an antigen or the passive exposure of the subject to the antigen in vivo. Methods for the active exposure of a subject to an antigen are well-known in the art. In general, an antigen is administered directly to the subject by any means such as intravenous, intramuscular, oral, transdermal, mucosal, intranasal, intratracheal, or subcutaneous administration. The antigen can be administered systemically or locally. Methods for administering the antigen and the imidazoquinoline agents are described in more detail below.

A subject is passively exposed to an antigen if an antigen becomes available for exposure to the immune cells in the body. A subject may be passively exposed to an antigen, for instance, by entry of a foreign pathogen into the body or by the development of a tumor cell expressing a foreign antigen on its surface.

The methods in which a subject is passively exposed to an antigen can be particularly dependent on timing of administration of the imidazoquinoline agents. For instance, in a subject at risk of developing a cancer or an infectious disease or an allergic or asthmatic response, the subject may be administered the imidazoquinoline agents on a regular basis when that risk is greatest, i.e., during allergy season or after exposure to a cancer causing agent. Additionally the imidazoquinoline agents may be administered to travelers before they travel to foreign lands where they are at risk of exposure to infectious agents. Likewise the imidazoquinoline agents may be administered to soldiers or civilians at risk of exposure to biowarfare to induce a systemic or mucosal immune response to the antigen when and if the subject is exposed to it.

In some cases it is desirable to administer an antigen with the imidazoquinoline agent and in other cases no antigen is delivered. An antigen is a molecule capable of provoking an immune response. The term antigen broadly includes any type of molecule that is recognized by a host system as being foreign. Antigens include but are not limited to microbial antigens, cancer antigens, and allergens.

Antigens include, but are not limited to, cells, cell extracts, proteins, polypeptides, peptides, polysaccharides, polysaccharide conjugates, peptide and non-peptide mimics of polysaccharides and other molecules, small molecules, lipids, glycolipids, and carbohydrates. Many antigens are protein or polypeptide in nature, as proteins and polypeptides are generally more antigenic than carbohydrates or fats.

The term substantially purified as used herein refers to a polypeptide which is substantially free of other proteins, lipids, carbohydrates or other materials with which it is naturally associated. One skilled in the art can purify viral or bacterial polypeptides using standard techniques for protein purification. The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the viral or bacterial polypeptide can also be determined by amino-terminal amino acid sequence analysis. Other types of antigens not encoded by a nucleic acid vector such as polysaccharides, small molecule, mimics etc are described above, and included within the invention.

A microbial antigen as used herein is an antigen of a microorganism and includes but is not limited to virus, bacteria, parasites, and fungi. Such antigens include the intact organism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

Polypeptides of bacterial pathogens include but are not limited to an iron-regulated outer membrane protein, (IROMP), an outer membrane protein (OMP), and an A-protein of Aeromonis salmonicida which causes furunculosis, p57 protein of Renibacterium salmoninarum which causes bacterial kidney disease (BKD), major surface associated antigen (msa), a surface expressed cytotoxin (mpr), a surface expressed hemolysin (ish), and a flagellar antigen of Yersiniosis; an extracellular protein (ECP), an iron-regulated outer membrane protein (IROMP), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia.

Polypeptides of a parasitic pathogen include but are not limited to the surface antigens of Ichthyophthirius.

Other microbial antigens that can be used together with the imidazoquinoline agents are provided in U.S. Non Provisional patent application Ser. No. 09/801,839, filed Mar. 8, 2001.

A cancer antigen as used herein is a compound, such as a peptide or protein, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

The terms “cancer antigen” and “tumor antigen” are used interchangeably and refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Examples of tumor antigens include MAGE, MART-1/Melan-A, gp100, Dipeptidyl peptidase IV (DPPIV), adenosine deaminase-binding protein (ADAbp), cyclophilin b, Colorectal associated antigen (CRC)—C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, alpha-fetoprotein, E-cadherin, alpha-catenin, beta-catenin and gamma-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

Cancers or tumors and tumor-antigens associated with such tumors (but not exclusively), include acute lymphoblastic leukemia (etv6; aml1; cyclophilin b), B cell lymphoma (Ig-idiotype), glioma (E-cadherin; alpha-catenin; beta-catenin; gamma-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)—C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkins lymphoma (lmp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (lmp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins), testicular cancer (NY-ESO-1), T cell leukemia (HTLV-1 epitopes), and melanoma (Melan-A/MART-1; cdc27; MAGE-3; p21ras; gp100^{Pmel117}).

Examples of tumor antigens which bind to either or both MHC class I and MHC class II molecules are known in the art. These antigens as well as others are disclosed in PCT Application PCT/US98/18601.

Other cancer antigens that can be used together with the imidazoquinoline agents are provided in U.S. Non-Provisional patent application Ser. No.09/800,266, filed Mar. 5, 2001.

An “allergen” as used herein is a molecule capable of provoking an immune response characterized by production of IgE. An allergen is a substance that can induce an allergic or asthmatic response in a susceptible subject. Thus, in the context of this invention, the term allergen means a specific type of antigen which can trigger an allergic response which is mediated by IgE antibody. The method and preparations of this invention extend to a broad class of such allergens and fragments of allergens or haptens acting as allergens. The list of allergens is enormous and can include pollens, insect venoms, animal dander dust, fungal spores and drugs (e.g. penicillin).

Other allergens that can be used together with the imidazoquinoline agents are provided in U.S. Non-Provisional patent application Ser. No.09/776, 479, filed Feb. 2, 2001.

The antigen may be an antigen that is encoded by a nucleic acid vector or it may not be encoded in a nucleic acid vector. In the former case the nucleic acid vector is administered to the subject and the antigen is expressed in vivo. In the latter case the antigen may be administered directly to the subject. An antigen not encoded in a nucleic acid vector as used herein refers to any type of antigen that is not a nucleic acid. For instance, in some aspects of the invention the antigen not encoded in a nucleic acid vector is a peptide or a polypeptide. Minor modifications of the primary amino acid sequences of peptide or polypeptide antigens may also result in a polypeptide which has substantially equivalent antigenic activity as compared to the unmodified counterpart polypeptide. Such modifications may be deliberate, as by site-directed mutagenesis, or may be spontaneous. All of the polypeptides produced by these modifications are included herein as long as antigenicity still exists. The peptide or polypeptide may be, for example, virally derived. The antigens useful in the invention may be any length, ranging from small peptide fragments of a full length protein or polypeptide to the full length form. For example, the antigen may be less than 5, less than 8, less than 10, less than 15, less than 20, less than 30, less than 50, less than 70, less than 100, or more amino acid residues in length, provided it stimulates a specific immune response when used in combination with the imidazoquinoline agents and/or other agents of the invention.

The nucleic acid encoding the antigen is operatively linked to a gene expression sequence which directs the expression of the antigen nucleic acid within a eukaryotic cell. The gene expression sequence is any regulatory nucleotide sequence, such as a promoter sequence or promoter-enhancer combination, which facilitates the efficient transcription and translation of the antigen nucleic acid to which it is operatively linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, b-actin promoter and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined antigen nucleic acid. The gene expression sequences optionally include enhancer sequences or upstream activator sequences as desired.

The antigen nucleic acid is operatively linked to the gene expression sequence. As used herein, the antigen nucleic acid sequence and the gene expression sequence are said to be operably linked when they are covalently linked in such a way as to place the expression or transcription and/or translation of the antigen coding sequence under the influence or control of the gene expression sequence. Two DNA sequences are said to be operably linked if induction of a promoter in the 5′ gene expression sequence results in the transcription of the antigen sequence and if the nature of the linkage between the two DNA sequences does not (1) result in the introduction of a frame-shift mutation, (2) interfere with the ability of the promoter region to direct the transcription of the antigen sequence, or (3) interfere with the ability of the corresponding RNA transcript to be translated into a protein. Thus, a gene expression sequence would be operably linked to an antigen nucleic acid sequence if the gene expression sequence were capable of effecting transcription of that antigen nucleic acid sequence such that the resulting transcript is translated into the desired protein or polypeptide.

The antigen nucleic acid of the invention may be delivered to the immune system alone or in association with a vector. In its broadest sense, a vector is any vehicle capable of facilitating the transfer of the antigen nucleic acid to the cells of the immune system so that the antigen can be expressed and presented on the surface of the immune cell. The vector generally transports the nucleic acid to the immune cells with reduced degradation relative to the extent of degradation that would result in the absence of the vector. The vector optionally includes the above-described gene expression sequence to enhance expression of the antigen nucleic acid in immune cells. In general, the vectors useful in the invention include, but are not limited to, plasmids, phagemids, viruses, other vehicles derived from viral or bacterial sources that have been manipulated by the insertion or incorporation of the antigen nucleic acid sequences. Viral vectors are a preferred type of vector and include, but are not limited to, nucleic acid sequences from the following viruses: retrovirus, such as Moloney murine leukemia virus, Harvey murine sarcoma virus, murine mammary tumor virus, and Rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known in the art.

Preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., Gene Transfer and Expression, A Laboratory Manual W. H. Freeman C. O., New York (1990) and Murray, E. J. Methods in Molecular Biology, vol. 7, Humana Press, Inc., Cliffton, N.J. (1991).

A preferred virus for certain applications is the adeno-associated virus, a double-stranded DNA virus. The adeno-associated virus can be engineered to be replication -deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, wild-type adeno-associated virus manifest some preference for integration sites into human cellular DNA, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Recombinant adeno-associated viruses that lack the replicase protein apparently lack this integration sequence specificity.

Other vectors include plasmid vectors. Plasmid vectors have been extensively described in the art and are well-known to those of skill in the art. See e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC18, pUC19, pRc/CMV, SV40, and pBlueScript. Other plasmids are well-known to those of ordinary skill in the art. Additionally, plasmids may be custom designed using restriction enzymes and ligation reactions to remove and add specific fragments of DNA.

It has recently been discovered that gene carrying plasmids can be delivered to the immune system using bacteria. Modified forms of bacteria such as Salmonella can be transfected with the plasmid and used as delivery vehicles. The bacterial delivery vehicles can be administered to a host subject orally or by other administration means. The bacteria deliver the plasmid to immune cells, e.g. B cells, dendritic cells, likely by passing through the gut barrier. High levels of immune protection have been established using this methodology. Such methods of delivery are useful for the aspects of the invention utilizing systemic delivery of antigen, imidazoquinoline agents and/or other therapeutic agent.

In some aspects of the invention, the imidazoquinoline agents are administered along with therapeutic agents such as disorder-specific medicaments. As used herein, a disorder-specific medicament is a therapy or agent that is used predominately in the treatment or prevention of a disorder. In one aspect, the imidazoquinoline agents may be administered to a subject with an anti-microbial agent. An anti-microbial agent, as used herein, refers to a naturally-occurring or synthetic compound which is capable of killing or inhibiting infectious organisms. The type of anti-microbial agent useful according to the invention will depend upon the type of organism with which the subject is infected or at risk of becoming infected.

In one aspect, the invention provides a method for treating or preventing a disorder. The method involves the administration of a synergistic combination of an imidazoquinoline agent and a disorder-specific medicament in an effective amount to prevent or treat the disorder to a subject having in need of such treatment.

In one aspect, the combination of imidazoquinoline agents and disorder-specific such treatment medicaments allows for the administration of higher doses of disorder-specific medicaments without as, many side effects as are ordinarily experienced at those high doses. In another aspect, the combination of imidazoquinoline agents and disorder-specific medicaments allows for the administration of lower, sub-therapeutic doses of either compound, but with higher efficacy than would otherwise be achieved using such low doses. As one example, by administering a combination of an imidazoquinoline agent and a medicament, it is possible to achieve an effective response even though the medicament is administered at a dose which alone would not provide a therapeutic benefit (i.e., a sub-therapeutic dose). As another example, the combined administration achieves a response even though the imidazoquinoline agent is administered at a dose which alone would not provide a therapeutic benefit.

The imidazoquinoline agents can also be administered on fixed schedules or in different temporal relationships to one another. The various combinations have many advantages over the prior art methods of modulating immune responses or preventing or treating disorders, particularly with regard to decreased non-specific toxicity to normal tissues.

The invention encompasses the administration of the imidazoquinoline agents along with a disorder-specific medicament in order to provide a synergistic effect useful in the prevention and/or treatment of a disorder. The beneficial effects of the imidazoquinoline agents are due, in part, to the modulation and stimulation of Th1 immune responses by these agents. The imidazoquinolines of the invention may provide the synergistic response via a number of mechanisms, including but not so limited to stimulation of hemopoietic recovery during or following cancer therapy, anti-microbial infection activity, enhancement of uptake of disorder-specific medicaments by immune cells and non-immune cells (depending upon the nature of the medicament), and inhibition or prevention of allergic responses to allergens in general and more specifically to the medicament.

The imidazoquinoline agents function to enhance defense mechanisms against bacterial, fungal, parasitic and viral infections. The prevention and control of such infections in immunocompromised cancer patients is a major challenge in the treatment and management of the disease. Such infections can usually disadvantageously delay or alter the course of treatment for cancer patients. The cellular and humoral immune responses stimulated by the nucleic acids reflect the body's own natural defense system against invading pathogens. The imidazoquinoline agents perform this function through the activation of innate immunity which is known to be most effective in the elimination of microbial infections. Enhancement of innate immunity occurs, inter alia, via increased IFN-alpha production and increased NK cell activity, both of which are effective in the treatment of microbial infections. The imidazoquinoline agents also function by enhancement of antibody-dependent cell cytotoxicity. This latter mechanism provides long-lasting effects of the nucleic acids, thereby reducing dosing regimes, improving compliance and maintenance therapy, reducing emergency situations; and improving quality of life. Some examples of common opportunistic infections in cancer patients are caused by Listeria monocytogenes, Pneumocystis carinii, cytomegalovirus, Mycobacterium tuberculosis, Staphylococcus aureus, Streptococcus pneumoniae, Haemophilus influenzae, Escherichia coli, Klebsiella pneumoniae, Pseudomonas aeruginosa, Nocardia, Candida, Aspergillus, and herpes viruses such as herpes simplex virus.

It is sometimes the case that subjects undergoing cancer treatment experience an adverse allergic reaction to the cancer medicament formulation being administered. The reaction may be specific to the cancer medicament itself or to other substances included in the cancer medicament formulation (e.g., the carrier substance, stabilizing agents, or sterilizing agents within the formulation). An example of a medicament which often triggers an allergic reaction upon administration is a formulation of Taxol. Such a reaction makes the use of such a medicament less desirable, and at the very least, may lead to the administration of the medicament at lower than therapeutic doses in order to avoid the allergic reaction. The present invention provides a method for avoiding such an adverse reaction through the administration of an imidazoquinoline agent. Reducing or eliminating the allergic reaction altogether may also allow for administration of disorder-specific medicaments in doses greater than the therapeutic dose, or at least greater than the doses currently administered.

The imidazoquinoline agents of the invention are also useful in the regulation of adverse allergic reactions in subjects undergoing transfusions. Subjects undergoing cancer treatment often require transfusions of red cells and/or platelets. Either due to incomplete separation of these cell types from others or due to differences in minor histocompatibility loci between the donor and the recipient of these blood products, subjects being infused may experience an acute allergic reaction to the transfusion. To counter this reaction which is primarily a Th2 type response, patients are administered allergy medication such as anti-histamines. Since imidazoquinoline agents elicit a Th1 response the subject may be administered an imidazoquinoline agent prior to or at the time of the transfusion in order to prevent or diminish the Th2 allergic reaction which might otherwise occur.

The imidazoquinoline agents when combined with the asthma/allergy medicaments have many advantages over each composition alone for the treatment of asthma and allergy. The imidazoquinoline agent functions in some aspects by simultaneously suppressing Th2-type immune responses (IL-4, IgE production, histamine release) that can result in airway inflammation and bronchial spasm, and/or inducing Th1-type immune responses (IFN-gamma and IL-12 production) that promote harmless antibody and cellular responses. This creates an environment inside the body that safely and effectively prevents hypersensitive reactions from occurring, thereby eliminating symptoms.

The imidazoquinoline agents when used in the methods of the invention can eliminate/reduce bronchial hyper-reactivity, bronchoconstriction, bronchial obstruction, airway inflammation and atopy (which improves asthma control, normalizes lung function, prevents irreversible airway injury); and may also inhibit acute response to exercise, cold dry air, and SO₂ The imidazoquinoline agents provide long-lasting effects, thus reducing dosing regimes, improving compliance and maintenance therapy, reducing emergency situations; and improving quality of life. These compounds are also useful because they provide early anti-infective activity, which leads to decreasing infectious episodes, which further reduces hyper-reactive immune responses. This is especially true in subjects like children or immuno-compromised subjects. Furthermore, use of the imidazoquinoline agents reduces/eliminates use of inhalers, which can exacerbate hypersensitive reactions by providing simpler and safer delivery and by allowing less drugs to be used.

Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents, which can be isolated from natural sources or synthesized, are useful for killing or inhibiting viruses. Anti-fungal agents are used to treat superficial fungal infections as well as opportunistic and primary systemic fungal infections. Anti-parasite agents kill or inhibit parasites.

One of the problems with anti-infective therapies is the side effects occurring in the host that is treated with the anti-infective. For instance, many anti-infectious agents can kill or inhibit a broad spectrum of microorganisms and are not specific for a particular type of species. Treatment with these types of anti-infectious agents results in the killing of the normal microbial flora living in the host, as well as the infectious microorganism. The loss of the microbial flora can lead to disease complications and predispose the host to infection by other pathogens, since the microbial flora compete with and function as barriers to infectious pathogens. Other side effects may arise as a result of specific or non-specific effects of these chemical entities on non-microbial cells or tissues of the host.

Another problem with wide-spread use of anti-infectants is the development of antibiotic resistant strains of microorganisms. Already, vancomycin-resistant enterococci, penicillin-resistant pneumococci, multi-resistant S. aureus, and multi-resistant tuberculosis strains have developed and are becoming major clinical problems. Widespread use of anti-infectants will likely produce many antibiotic-resistant strains of bacteria. As a result, new anti-infective strategies will be required to combat these microorganisms.

A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics.

Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors. Cell wall synthesis inhibitors inhibit a step in the process of cell wall synthesis, and in general in the synthesis of bacterial peptidoglycan. Cell wall synthesis inhibitors include beta-lactam antibiotics, natural penicillins, semi-synthetic penicillins, ampicillin, clavulanic acid, cephalolsporins, and bacitracin.

The beta-lactams are antibiotics containing a four-membered beta-lactam ring which inhibits the last step of peptidoglycan synthesis. The beta-lactam antibiotics produced by penicillium are the natural penicillins, such as penicillin G or penicillin V. The natural penicillins have a narrow spectrum of activity and are generally effective against Streptococcus, Gonococcus, and Staphylococcus. Other types of natural penicillins, which are also effective against gram-positive bacteria, include penicillins F, X, K, and O.

Semi-synthetic penicillins are generally modifications of the molecule 6-aminopenicillanic acid produced by a mold. The 6-aminopenicillanic acid can be modified by addition of side chains which produce penicillins having broader spectrums of activity than natural penicillins or various other advantageous properties. Some types of semi-synthetic penicillins have broad spectrums against gram-positive and gram-negative bacteria, but are inactivated by penicillinase. These semi-synthetic penicillins include ampicillin, carbenicillin, oxacillin, azlocillin, mezlocillin, and piperacillin. Other types of semi-synthetic penicillins have narrower activities against gram-positive bacteria, but have developed properties such that they are not inactivated by penicillinase. These include, for instance, methicillin, dicloxacillin, and nafcillin. Some of the broad spectrum semi-synthetic penicillins can be used in combination with beta-lactamase inhibitors, such as clavulamic acids and sulbactam. The beta-lactamase inhibitors do not have anti-microbial action but they function to inhibit penicillinase, thus protecting the semi-synthetic penicillin from degradation.

One of the serious side effects associated with penicillins, both natural and semi-synthetic, is penicillin-allergy. Penicillin allergies are very serious and can cause death rapidly. In a subject that is allergic to penicillin, the beta-lactam molecule will attach to a serum protein which initiates an IgE-mediated inflammatory response. The inflammatory response leads to anaphylaxis and possibly death.

Another type of beta-lactam antibiotic is the cephalolsporins. They are sensitive to degradation by bacterial beta-lactamases, and thus, are not always effective alone. Cephalolsporins, however, are resistant to penicillinase. They are effective against a variety of gram-positive and gram-negative bacteria. Cephalolsporins include, but are not limited to, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, and moxalactam.

Bacitracin is another class of antibiotics which inhibit cell wall synthesis. Although bacitracin is effective against gram-positive bacteria, its use is limited in general to topical administration because of its high toxicity. Since lower effective doses of bacitracen can be used when the compound is administered with the imidazoquinoline agents of the invention, this compound can be used systemically and the toxicity reduced.

Carbapenems are another broad spectrum beta-lactam antibiotic, which is capable of inhibiting cell wall synthesis. Examples of carbapenems include, but are not limited to, imipenems. Monobactems are also broad spectrum beta-lactam antibiotics, and include, euztreonam. An antibiotic produced by streptomyces, vancomycin, is also effective against gram-positive bacteria by inhibiting cell membrane synthesis.

Another class of anti-bacterial agents is the anti-bacterial agents that are cell membrane inhibitors. These compounds disorganize the structure or inhibit the function of bacterial membranes. One problem with anti-bacterial agents that are cell membrane inhibitors is that they can produce effects in eukaryotic cells as well as bacteria because of the similarities in phospholipids in bacterial and eukaryotic membranes. Thus these compounds are rarely specific enough to permit these compounds to be used systemically and prevent the use of high doses for local administration.

One clinically cell membrane inhibitor is Polymyxin. Polymyxin is effective mainly against Gram-negative bacteria and is generally used in severe Pseudomonas infections or Pseudomonas infections that are resistant to less toxic antibiotics. The severe side effects associated with systemic administration of this compound include damage to the kidney and other organs.

Other cell membrane inhibitors include Amphotericin B and Nystatin which are also anti-fungal agents used predominantly in the treatment of systemic fungal infections and Candida yeast infections respectively. Imidazoles are another class of antibiotic that is a cell membrane inhibitor. Imidazoles are used as bacterial agents as well as anti-fungal agents, e.g., used for treatment of yeast infections, dermatophytic infections, and systemic fungal infections. Imidazoles include but are not limited to clotrimazole, miconazole, ketoconazole, itraconazole, and fluconazole.

Many anti-bacterial agents are protein synthesis inhibitors. These compounds prevent bacteria from synthesizing structural proteins and enzymes and thus cause inhibition of bacterial cell growth or function or cell death. Anti-bacterial agents that block transcription include but are not limited to Rifampins and Ethambutol. Rifampins, which inhibit the enzyme RNA polymerase, have a broad spectrum activity and are effective against gram-positive and gram-negative bacteria as well as Mycobacterium tuberculosis. Ethambutol is effective against Mycobacterium tuberculosis.

Anti-bacterial agents which block translation include but are not limited to tetracyclines, chloramphenicol, the macrolides (e.g., erythromycin) and the aminoglycosides (e.g., streptomycin).

The aminoglycosides are a class of antibiotics which are produced by the bacterium Streptomyces, such as, for instance streptomycin, kanamycin, tobramycin, amikacin, and gentamicin. Aminoglycosides have been used against a wide variety of bacterial infections caused by Gram-positive and Gram-negative bacteria. Streptomycin has been used extensively as a primary drug in the treatment of tuberculosis. Gentamicin is used against many strains of Gram-positive and Gram-negative bacteria, including Pseudomonas infections, especially in combination with Tobramycin. Kanamycin is used against many Gram-positive bacteria, including penicillin-resistant Staphylococci. One side effect of aminoglycosides that has limited their use clinically is that at dosages which are essential for efficacy, prolonged use has been shown to impair kidney function and cause damage to the auditory nerves leading to deafness.

Another type of translation inhibitor anti-bacterial agent is the tetracyclines. The tetracyclines are a class of antibiotics that are broad-spectrum and are effective against a variety of gram-positive and gram-negative bacteria. Examples of tetracyclines include tetracycline, minocycline, doxycycline, and chlortetracycline. They are important for the treatment of many types of bacteria but are particularly important in the treatment of Lyme disease. As a result of their low toxicity and minimal direct side effects, the tetracyclines have been overused and misused by the medical community, leading to problems. For instance, their overuse has led to wide-spread development of resistance. When used in combination with the imidazoquinoline agents of the invention, these problems can be minimized and tetracyclines can be effectively used for the broad spectrum treatment of many bacteria.

Anti-bacterial agents such as the macrolides bind reversibly to the 50s ribosomal subunit and inhibit elongation of the protein by peptidyl transferase or prevent the release of uncharged tRNA from the bacterial ribosome or both. These compounds include erythromycin, roxithromycin, clarithromycin, oleandomycin, and azithromycin. Erythromycin is active against most Gram-positive bacteria, Neisseria, Legionella and Haemophilus, but not against the Enterobacteriaceae. Lincomycin and clindamycin, which block peptide bond formation during protein synthesis, are used against gram-positive bacteria.

Another type of translation inhibitor is chloramphenicol. Chloramphenicol binds the 70S ribosome inhibiting the bacterial enzyme peptidyl transferase thereby preventing the growth of the polypeptide chain during protein synthesis. One serious side effect associated with chloramphenicol is aplastic anemia. Aplastic anemia develops at doses of chloramphenicol which are effective for treating bacteria in a small proportion (1/50,000) of patients. Chloramphenicol which was once a highly prescribed antibiotic is now seldom uses as a result of the deaths from anemia. Because of its effectiveness it is still used in life-threatening situations (e.g. typhoid fever). By combining chloramphenicol with the imidazoquinoline agents these compounds can again be used as anti-bacterial agents because the immunostimulatory agents allow a lower dose of the chloramphenicol to be used, a dose that does not produce side effects.

Some anti-bacterial agents disrupt nucleic acid synthesis or function, e.g., bind to DNA or RNA so that their messages cannot be read. These include but are not limited to quinolones and co-trimoxazole, both synthetic chemicals and rifamycins, a natural or semi-synthetic chemical. The quinolones block bacterial DNA replication by inhibiting the DNA gyrase, the enzyme needed by bacteria to produce their circular DNA. They are broad spectrum and examples include norfloxacin, ciprofloxacin, enoxacin, nalidixic acid and temafloxacin. Nalidixic acid is a bactericidal agent that binds to the DNA gyrase enzyme (topoisomerase) which is essential for DNA replication and allows supercoils to be relaxed and reformed, inhibiting DNA gyrase activity. The main use of nalidixic acid is in treatment of lower urinary tract infections (UTI) because it is effective against several types of Gram-negative bacteria such as E. coli, Enterobacter aerogenes, K. pneumoniae and Proteus species which are common causes of UTI. Co-trimoxazole is a combination of sulfamethoxazole and trimethoprim, which blocks the bacterial synthesis of folic acid needed to make DNA nucleotides. Rifampicin is a derivative of rifamycin that is active against Gram-positive bacteria (including Mycobacterium tuberculosis and meningitis caused by Neisseria meningitidis) and some Gram-negative bacteria. Rifampicin binds to the beta subunit of the polymerase and blocks the addition of the first nucleotide which is necessary to activate the polymerase, thereby blocking mRNA synthesis.

Another class of anti-bacterial agents is compounds that function as competitive inhibitors of bacterial enzymes. The competitive inhibitors are mostly all structurally similar to a bacterial growth factor and compete for binding but do not perform the metabolic function in the cell. These compounds include sulfonamides and chemically modified forms of sulfanilamide which have even higher and broader antibacterial activity. The sulfonamides (e.g. gantrisin and trimethoprim) are useful for the treatment of Streptococcus pneumoniae, beta-hemolytic streptococci and E. coli, and have been used in the treatment of uncomplicated UTI caused by E. coli, and in the treatment of meningococcal meningitis.

Other anti-bacterial agents that can be used in the methods and compositions of the invention are listed in U.S. Non-Provisional patent application Ser. No. 09/801,839, filed Mar. 8, 2001.

Anti-viral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogues), maturation of new virus proteins (e.g. protease inhibitors), and budding and release of the virus.

Another category of anti-viral agents are nucleotide analogues. Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine).

Another class of anti-viral agents are cytokines such as interferons. The interferons are cytokines which are secreted by virus-infected cells as well as immune cells. The interferons function by binding to specific receptors on cells adjacent to the infected cells, causing the change in the cell which protects it from infection by the virus. Alpha and beta-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Immunoglobulin therapy is used for the prevention of viral infection. Immunoglobulin therapy for viral infections is different than bacterial infections, because rather than being antigen-specific, the immunoglobulin therapy functions by binding to extracellular virions and preventing them from attaching to and entering cells which are susceptible to the viral infection. The therapy is useful for the prevention of viral infection for the period of time that the antibodies are present in the host. In general there are two types of immunoglobulin therapies, normal immunoglobulin therapy and hyper-immunoglobulin therapy. Normal immune globulin therapy utilizes a antibody product which is prepared from the serum of normal blood donors and pooled. This pooled product contains low titers of antibody to a wide range of human viruses, such as hepatitis A, parvovirus, enterovirus (especially in neonates). Hyper-immune globulin therapy utilizes antibodies which are prepared from the serum of individuals who have high titers of an antibody to a particular virus. Those antibodies are then used against a specific virus. Examples of hyper-immune globulins include zoster immune globulin (useful for the prevention of varicella in immuno-compromised children and neonates), human rabies immunoglobulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncitial virus infections).

Another type of immunoglobulin therapy is active immunization. This involves the administration of antibodies or antibody fragments to viral surface proteins. Two types of vaccines which are available for active immunization of hepatitis B include serum-derived hepatitis B antibodies and recombinant hepatitis B antibodies. Both are prepared from HBsAg. The antibodies are administered in three doses to subjects at high risk of infection with hepatitis B virus, such as health care workers, sexual partners of chronic carriers, and infants.

The combination of imidazoquinoline agents with immunoglobulin therapy also provides benefit via the ability of imidazoquinoline agents to enhance ADCC as discussed herein.

Other anti-viral agents that can be used in the methods and compositions of the invention are listed in U.S. Non-Provisional patent application Ser. No. 09/801,839, filed Mar. 8, 2001.

Anti-fungal agents are useful for the treatment and prevention of infective fungi. Anti-fungal agents are sometimes classified by their mechanism of action. Some anti-fungal agents function as cell wall inhibitors by inhibiting glucose synthase. Other anti-fungal agents function by destabilizing membrane integrity.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, imidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

Other anti-fungal agents that can be used in the methods and compositions of the invention are listed in U.S. Non-Provisional patent application Ser. No. 09/801,839, filed Mar. 8, 2001.

Anti-parasitic agents that can be used in the methods and compositions of the invention are listed in U.S. Non-Provisional patent application Ser. No. 09/306,281, filed May 6, 1999.

The imidazoquinoline agents may also be administered in conjunction with an anti-cancer therapy. Anti-cancer therapies include cancer medicaments, radiation and surgical procedures. As used herein, a “cancer medicament” refers to an agent which is administered to a subject for the purpose of treating a cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers.

Cancer is currently treated using a variety of modalities including surgery, radiation therapy and chemotherapy. The choice of treatment modality will depend upon the type, location and dissemination of the cancer. For example, surgery and radiation therapy may be more appropriate in the case of solid well-defined tumor masses and less practical in the case of non-solid tumor cancers such as leukemia and lymphoma. One of the advantages of surgery and radiation therapy is the ability to control to some extent the impact of the therapy, and thus to limit the toxicity to normal tissues in the body. However, surgery and radiation therapy are often followed by chemotherapy to guard against any remaining or radio-resistant cancer cells. Chemotherapy is also the most appropriate treatment for disseminated cancers such as leukemia and lymphoma as well as metastases.

Chemotherapy refers to therapy using chemical and/or biological agents to attack cancer cells. Unlike localized surgery or radiation, chemotherapy is generally administered in a systemic fashion and thus toxicity to normal tissues is a major concern. Because many chemotherapy agents target cancer cells based on their proliferative profiles, tissues such as the gastrointestinal tract and the bone marrow which are normally proliferative are also susceptible to the effects of the chemotherapy. One of the major side effects of chemotherapy is myelosuppression (including anemia, neutropenia and thrombocytopenia) which results from the death of normal hemopoietic precursors.

Many chemotherapeutic agents have been developed for the treatment of cancer. Not all tumors, however, respond to chemotherapeutic agents and others although initially responsive to chemotherapeutic agents may develop resistance. As a result, the search for effective anti-cancer drugs has intensified in an effort to find even more effective agents with less non-specific toxicity.

Cancer medicaments function in a variety of ways. Some cancer medicaments work by targeting physiological mechanisms that are specific to tumor cells. Examples include the targeting of specific genes and their gene products (i.e., proteins primarily) which are mutated in cancers. Such genes include but are not limited to oncogenes (e.g., Ras, Her2, bcl-2), tumor suppressor genes (e.g., EGF, p53, Rb), and cell cycle targets (e.g., CDK4, p21, telomerase). Cancer medicaments can alternately target signal transduction pathways and molecular mechanisms which are altered in cancer cells. Targeting of cancer cells via the epitopes expressed on their cell surface is accomplished through the use of monoclonal antibodies. This latter type of cancer medicament is generally referred to herein as immunotherapy.

Other cancer medicaments target cells other than cancer cells. For example, some medicaments prime the immune system to attack tumor cells (i.e., cancer vaccines). Still other medicaments, called angiogenesis inhibitors, function by attacking the blood supply of solid tumors. Since the most malignant cancers are able to metastasize (i.e., exist the primary tumor site and seed a distal tissue, thereby forming a secondary tumor), medicaments that impede this metastasis are also useful in the treatment of cancer. Angiogenic mediators include basic FGF, VEGF, angiopoietins, angiostatin, endostatin, TNFα, TNP-470, thrombospondin-1, platelet factor 4, CAI, and certain members of the integrin family of proteins. One category of this type of medicament is a metalloproteinase inhibitor, which inhibits the enzymes used by the cancer cells to exist the primary tumor site and extravasate into another tissue.

Some cancer cells are antigenic and thus can be targeted by the immune system. In one aspect, the combined administration of imidazoquinoline agents and cancer medicaments, particularly those which are classified as cancer immunotherapies, is useful for stimulating a specific immune response against a cancer antigen.

The theory of immune surveillance is that a prime function of the immune system is to detect and eliminate neoplastic cells before a tumor forms. A basic principle of this theory is that cancer cells are antigenically different from normal cells and thus elicit immune reactions that are similar to those that cause rejection of immunologically incompatible allografts. Studies have confirmed that tumor cells differ, either qualitatively or quantitatively, in their expression of antigens. For example, “tumor-specific antigens” are antigens that are specifically associated with tumor cells but not normal cells. Examples of tumor specific antigens are viral antigens in tumors induced by DNA or RNA viruses. “Tumor-associated” antigens are present in both tumor cells and normal cells but are present in a different quantity or a different form in tumor cells. Examples of such antigens are oncofetal antigens (e.g., carcinoembryonic antigen), differentiation antigens (e.g., T and Tn antigens), and oncogene products (e.g., HER/neu).

Different types of cells that can kill tumor targets in vitro and in vivo have been identified: natural killer cells (NK cells), cytolytic T lymphocytes (CTLs), lymphokine-activated killer cells (LAKs), and activated macrophages. NK cells can kill tumor cells without having been previously sensitized to specific antigens, and the activity does not require the presence of class I antigens encoded by the major histocompatibility complex (MHC) on target cells. NK cells are thought to participate in the control of nascent tumors and in the control of metastatic growth. In contrast to NK cells, CTLs can kill tumor cells only after they have been sensitized to tumor antigens and when the target antigen is expressed on the tumor cells that also express MHC class I. CTLs are thought to be effector cells in the rejection of transplanted tumors and of tumors caused by DNA viruses. LAK cells are a subset of null lymphocytes distinct from the NK and CTL populations. Activated macrophages can kill tumor cells in a manner that is not antigen dependent nor MHC restricted once activated. Activated macrophages are through to decrease the growth rate of the tumors they infiltrate. In vitro assays have identified other immune mechanisms such as antibody-dependent, cell-mediated cytotoxic reactions and lysis by antibody plus complement. However, these immune effector mechanisms are thought to be less important in vivo than the function of NK, CTLs, LAK, and macrophages in vivo (for review see Piessens, W. F., and David, J., “Tumor Immunology”, In: Scientific American Medicine, Vol. 2, Scientific American Books, N.Y., pp. 1-13, 1996.

The goal of immunotherapy is to augment a patient's immune response to an established tumor. One method of immunotherapy includes the use of adjuvants. Adjuvant substances derived from microorganisms, such as bacillus Calmette-Guerin, heighten the immune response and enhance resistance to tumors in animals.

Immunotherapeutic agents are medicaments which derive from antibodies or antibody fragments which specifically bind or recognize a cancer antigen. Antibody-based immunotherapies may function by binding to the cell surface of a cancer cell and thereby stimulate the endogenous immune system to attack the cancer cell. Another way in which antibody-based therapy functions is as a delivery system for the specific targeting of toxic substances to cancer cells. Antibodies are usually conjugated to toxins such as ricin (e.g., from castor beans), calicheamicin and maytansinoids, to radioactive isotopes such as Iodine-131 and Yttrium-90, to chemotherapeutic agents (as described herein), or to biological response modifiers. In this way, the toxic substances can be concentrated in the region of the cancer and non-specific toxicity to normal cells can be minimized. In addition to the use of antibodies which are specific for cancer antigens, antibodies which bind to vasculature, such as those which bind to endothelial cells, are also useful in the invention. This is because generally solid tumors are dependent upon newly formed blood vessels to survive, and thus most tumors are capable of recruiting and stimulating the growth of new blood vessels. As a result, one strategy of many cancer medicaments is to attack the blood vessels feeding a tumor and/or the connective tissues (or stroma) supporting such blood vessels.

The use of imidazoquinoline agents in conjunction with immunotherapeutic agents such as monoclonal antibodies is able to increase long-term survival through a number of mechanisms including significant enhancement of ADCC (as discussed above), activation of natural killer (NK) cells and an increase in IFN alpha levels. The imidazoquinoline agents when used in combination with monoclonal antibodies serve to reduce the dose of the antibody required to achieve a biological result.

Cancer vaccines are medicaments which are intended to stimulate an endogenous immune response against cancer cells. Currently produced vaccines predominantly activate the humoral immune system (i.e., the antibody-dependent immune response). Other vaccines currently in development are focused on activating the cell-mediated immune system including cytotoxic T lymphocytes which are capable of killing tumor cells. Cancer vaccines generally enhance the presentation of cancer antigens to both antigen presenting cells (e.g., macrophages and dendritic cells) and/or to other immune cells such as T cells, B cells, and NK cells.

Although cancer vaccines may take one of several forms, as discussed infra, their purpose is to deliver cancer antigens and/or cancer associated antigens to antigen presenting cells (APC) in order to facilitate the endogenous processing of such antigens by APC and the ultimate presentation of antigen presentation on the cell surface in the context of MHC class I molecules. One form of cancer vaccine is a whole cell vaccine which is a preparation of cancer cells which have been removed from a subject, treated ex vivo and then reintroduced as whole cells in the subject. Lysates of tumor cells can also be used as cancer vaccines to elicit an immune response. Another form cancer vaccine is a peptide vaccine which uses cancer-specific or cancer-associated small proteins to activate T cells. Cancer-associated proteins are proteins which are not exclusively expressed by cancer cells (i.e., other normal cells may still express these antigens). However, the expression of cancer-associated antigens is generally consistently upregulated with cancers of a particular type. Yet another form of cancer vaccine is a dendritic cell vaccine which includes whole dendritic cells which have been exposed to a cancer antigen or a cancer-associated antigen in vitro. Lysates or membrane fractions of dendritic cells may also be used as cancer vaccines. Dendritic cell vaccines are able to activate antigen-presenting cells directly. Other cancer vaccines include ganglioside vaccines, heat-shock protein vaccines, viral and bacterial vaccines, and nucleic acid vaccines.

The use of imidazoquinoline agents in conjunction with cancer vaccines provides an improved antigen-specific humoral and cell mediated immune response, in addition to activating NK cells and endogenous dendritic cells, and increasing IFN alpha levels. This enhancement allows a vaccine with a reduced antigen dose to be used to achieve the same beneficial effect. In some instances, cancer vaccines may be used along with adjuvants, such as those described above.

Other vaccines take the form of dendritic cells (DCs) which have been exposed to cancer antigens in vitro, have processed the antigens and are able to express the cancer antigens at their cell surface in the context of MHC molecules for effective antigen presentation to other immune system cells. In one embodiment, the imidazoquinoline agent and the DC vaccine are mixed upon re-injection into a subject. Alternatively, the imidazoquinoline agent can be used in the in vitro preparation of the vaccine for example in the culture, maturation or activation of DCs. Monocytic DCs (mDCs) in particular can benefit from the combined use of imidazoquinoline agents. Synergy when using mixed populations of CDs (i.e., combinations of plasmacytoid DCs (pDCs) and mDCs) is also envisioned.

The imidazoquinoline agents are used in one aspect of the invention in conjunction with cancer vaccines which are dendritic cell based. A dendritic cell is a professional antigen presenting cell. Dendritic cells form the link between the innate and the acquired immune system by presenting antigens and through their expression of pattern recognition receptors which detect microbial molecules like LPS in their local environment. Dendritic cells efficiently internalize, process, and present soluble specific antigen to which it is exposed. The process of internalizing and presenting antigen causes rapid upregulation of the expression of major histocompatibility complex (MHC) and costimulatory molecules, the production of cytokines, and migration toward lymphatic organs where they are believed to be involved in the activation of T cells.

As used herein, chemotherapeutic agents embrace all other forms of cancer medicaments which do not fall into the categories of immunotherapeutic agents or cancer vaccines. Chemotherapeutic agents as used herein encompass both chemical and biological agents. These agents function to inhibit a cellular activity which the cancer cell is dependent upon for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. Combination chemotherapy and imidazoquinoline agent administration increases the maximum tolerable dose of chemotherapy.

Further examples of cancer medicaments that can be used in the methods and compositions of the present invention are listed in U.S. Non-Provisional patent application Ser. No. 09/800,266, filed Mar. 5, 2001.

The imidazoquinoline agents may also be administered in conjunction with an asthma or allergy medicament. An “asthma/allergy medicament” as used herein is a composition of matter which reduces the symptoms, inhibits the asthmatic or allergic reaction, or prevents the development of an allergic or asthmatic reaction. Various types of medicaments for the treatment of asthma and allergy are described in the Guidelines For The Diagnosis and Management of Asthma, Expert Panel Report 2, NIH Publication No. 97/4051, Jul. 19, 1997, the entire contents of which are incorporated herein by reference. The summary of the medicaments as described in the NIH publication is presented below. In most embodiments the asthma/allergy medicament is useful to some degree for treating both asthma and allergy.

Medications for the treatment of asthma are generally separated into two categories, quick-relief medications and long-term control medications. Asthma patients take the long-term control medications on a daily basis to achieve and maintain control of persistent asthma. Long-term control medications include anti-inflammatory agents such as corticosteroids, chromolyn sodium and medacromil; long-acting bronchodilators, such as long-acting β₂-agonists and methylxanthines; and leukotriene modifiers. The quick-relief medications include short-acting β₂ agonists, anti-cholinergics, and systemic corticosteroids. There are many side effects associated with each of these drugs and none of the drugs alone or in combination is capable of preventing or completely treating asthma.

Asthma medicaments include, but are not limited, PDE-4 inhibitors, Bronchodilator/beta-2 agonists, K+ channel openers, VLA-4 antagonists, Neurokin antagonists, TXA2 synthesis inhibitors, Xanthanines, Arachidonic acid antagonists, 5 lipoxygenase inhibitors, Thromboxin A2 receptor antagonists, Thromboxane A2 antagonists, Inhibitor of 5-lipox activation proteins, and Protease inhibitors.

Bronchodilator/beta-2 agonists are a class of compounds which cause bronchodilation or smooth muscle relaxation. Bronchodilator/beta-2 agonists include, but are not limited to, salmeterol, salbutamol, albuterol, terbutaline, D2522/formoterol, fenoterol, bitolterol, pirbuerol methylxanthines and orciprenaline. Long-acting β₂ agonists and bronchodilators are compounds which are used for long-term prevention of symptoms in addition to the anti-inflammatory therapies. Long-acting β₂ agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. Dose-related acute toxicities are a particular problem with these types of compounds. As a result, routine serum concentration must be monitored in order to account for the toxicity and narrow therapeutic range arising from individual differences in metabolic clearance. Side effects include tachycardia, nausea and vomiting, tachyarrhythmias, central nervous system stimulation, headache, seizures, hematemesis, hyperglycemia and hypokalemia. Short-acting β₂ agonists include, but are not limited to, albuterol, bitolterol, pirbuterol, and terbutaline. Some of the adverse effects associated with the administration of short-acting β₂ agonists include tachycardia, skeletal muscle tremor, hypokalemia, increased lactic acid, headache, and hyperglycemia.

Conventional methods for treating or preventing allergy have involved the use of anti-histamines or desensitization therapies. Anti-histamines and other drugs which block the effects of chemical mediators of the allergic reaction help to regulate the severity of the allergic symptoms but do not prevent the allergic reaction and have no effect on subsequent allergic responses. Desensitization therapies are performed by giving small doses of an allergen, usually by injection under the skin, in order to induce an IgG-type response against the allergen. The presence of IgG antibody helps to neutralize the production of mediators resulting from the induction of IgE antibodies, it is believed. Initially, the subject is treated with a very low dose of the allergen to avoid inducing a severe reaction and the dose is slowly increased. This type of therapy is dangerous because the subject is actually administered the compounds which cause the allergic response and severe allergic reactions can result.

Allergy medicaments include, but are not limited to, anti-histamines, steroids, and prostaglandin inducers. Anti-histamines are compounds which counteract histamine released by mast cells or basophils. These compounds are well known in the art and commonly used for the treatment of allergy. Anti-histamines include, but are not limited to, loratidine, cetirizine, buclizine, ceterizine analogues, fexofenadine, terfenadine, desloratadine, norastemizole, epinastine, ebastine, ebastine, astemizole, levocabastine, azelastine, tranilast, terfenadine, mizolastine, betatastine, CS 560, and HSR 609. Prostaglandin inducers are compounds which induce prostaglandin activity. Prostaglandins function by regulating smooth muscle relaxation. Prostaglandin inducers include, but are not limited to, S-5751.

The asthma/allergy medicaments useful in combination with the imidazoquinoline agents also include steroids and immunomodulators. The steroids include, but are not limited to, beclomethasone, fluticasone, tramcinolone, budesonide, corticosteroids and budesonide.

Corticosteroids include, but are not limited to, beclomethasome dipropionate, budesonide, flunisolide, fluticaosone, propionate, and triamcinoone acetonide. Although dexamethasone is a corticosteroid having anti-inflammatory action, it is not regularly used for the treatment of asthma/allergy in an inhaled form because it is highly absorbed, it has long-term suppressive side effects at an effective dose. Dexamethasone, however, can be used according to the invention for the treating of asthma/allergy because when administered in combination with imidazoquinoline agents it can be administered at a low dose to reduce the side effects. Additionally, the imidazoquinoline agents can be administered to reduce the side effects of dexamethasone at higher concentrations. Some of the side effects associated with corticosteroid include cough, dysphonia, oral thrush (candidiasis), and in higher doses, systemic effects, such as adrenal suppression, osteoporosis, growth suppression, skin thinning and easy bruising. (Barnes & Peterson, Am. Rev. Respir. Dis.; 148:S1-S26, 1993; and Kamadaet al., Am. J. Respir. Crit Care Med.; 153:1739-48, 1996).

Systemic corticosteroids include, but are not limited to, methylprednisolone, prednisolone and prednisone. Cortosteroids are associated with reversible abnormalities in glucose metabolism, increased appetite, fluid retention, weight gain, mood alteration, hypertension, peptic ulcer, and rarely asceptic necrosis of femur. These compounds are useful for short-term (3-10 days) prevention of the inflammatory reaction in inadequately controlled persistent asthma. They also function in a long-term prevention of symptoms in severe persistent asthma to suppress and control and actually reverse inflammation. Some side effects associated with longer term use include adrenal axis suppression, growth suppression, dermal thinning, hypertension, diabetes, Cushing's syndrome, cataracts, muscle weakness, and in rare instances, impaired immune function. It is recommended that these types of compounds be used at their lowest effective dose (guidelines for the diagnosis and management of asthma; expert panel report to; NIH Publication No. 97-4051; July 1997).

The immunomodulators include, but are not limited to, the group consisting of anti-inflammatory agents, leukotriene antagonists, IL-4 muteins, soluble IL-4 receptors, immunosuppressants (such as tolerizing peptide vaccine), anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and, and downregulators of IgE.

Leukotriene modifiers are often used for long-term control and prevention of symptoms in mild persistent asthma. Leukotriene modifiers function as leukotriene receptor antagonists by selectively competing for LTD-4 and LTE-4 receptors. These compounds include, but are not limited to, zafirlukast tablets and zileuton tablets. Zileuton tablets function as 5-lipoxygenase inhibitors. These drugs have been associated with the elevation of liver enzymes and some cases of reversible hepatitis and hyperbilirubinemia. Leukotrienes are biochemical mediators that are released from mast cells, eosinophils, and basophils that cause contraction of airway smooth muscle and increase vascular permeability, mucous secretions and activate inflammatory cells in the airways of patients with asthma.

Other immunomodulators include neuropeptides that have been shown to have immunomodulating properties. Functional studies have shown that substance P, for instance, can influence lymphocyte function by specific receptor mediated mechanisms. Substance P also has been shown to modulate distinct immediate hypersensitivity responses by stimulating the generation of arachidonic acid-derived mediators from mucosal mast cells. J. McGillies, et al., Substance P and Immunoregulation, Fed. Proc. 46:196-9 (1987). Substance P is a neuropeptide first identified in 1931 by Von Euler and Gaddum. An unidentified depressor substance in certain tissue extracts, J. Physiol. (London) 72:74-87 (1931). Its amino acid sequence, Arg-Pro-Lys-Pro-Gln-Gln-Phe-Phe-Gly-Leu-Met-NH.sub.2 (Sequence Id. No. 1) was reported by Chang et al. in 1971. Amino acid sequence of substance P, Nature (London) New Biol. 232:86-87 (1971). The immunoregulatory activity of fragments of substance P has been studied by Siemion, et al. Immunoregulatory Activity of Substance P Fragments, Molec. Immunol. 27:887-890 (1990).

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. Anti-IgE is being developed by Genentech. One of skill in the art could prepare functionally active antibody fragments of binding peptides which have the same function. Other types of IgE downregulators are polypeptides capable of blocking the binding of the IgE antibody to the Fc receptors on the cell surfaces and displacing IgE from binding sites upon which IgE is already bound.

One problem associated with downregulators of IgE is that many molecules do not have a binding strength to the receptor corresponding to the very strong interaction between the native IgE molecule and its receptor. The molecules having this strength tend to bind irreversibly to the receptor. However, such substances are relatively toxic since they can bind covalently and block other structurally similar molecules in the body. Of interest in this context is that the a chain of the IgE receptor belongs to a larger gene family where i.e. several of the different IgG Fe receptors are contained. These receptors are absolutely essential for the defense of the body against i.e. bacterial infections. Molecules activated for covalent binding are, furthermore, often relatively unstable and therefore they probably have to be administered several times a day and then in relatively high concentrations in order to make it possible to block completely the continuously renewing pool of IgE receptors on mast cells and basophilic leukocytes.

These types of asthma/allergy medicaments are sometimes classified as long-term control medications or quick-relief medications. Long-term control medications include compounds such as corticosteroids (also referred to as glucocorticoids), methylprednisolone, prednisolone, prednisone, cromolyn sodium, nedocromil, long-acting β₂-agonists, methylxanthines, and leukotriene modifiers. Quick relief medications are useful for providing quick relief of symptoms arising from allergic or asthmatic responses. Quick relief medications include short-acting β₂ agonists, anticholinergics and systemic corticosteroids.

Chromolyn sodium and medocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from eosinophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratrapoium bromide. These compounds reverse only cholinerigically-mediated bronchospasm and do not modify any reaction to antigen. Side effects include drying of the mouth and respiratory secretions, increased wheezing in some individuals, blurred vision if sprayed in the eyes.

In addition to standard asthma/allergy medicaments other methods for treating asthma/allergy have been used either alone or in combination with established medicaments. One preferred, but frequently impossible, method of relieving allergies is allergen or initiator avoidance. Another method currently used for treating allergic disease involves the injection of increasing doses of allergen to induce tolerance to the allergen and to prevent further allergic reactions.

Allergen injection therapy (allergen immunotherapy) is known to reduce the severity of allergic rhinitis. This treatment has been theorized to involve the production of a different form of antibody, a protective antibody which is termed a “blocking antibody”. Cooke, RA et al., Serologic Evidence of Immunity with Coexisting Sensitization in a Type of Human Allergy, Exp. Med. 62:733 (1935). Other attempts to treat allergy involve modifying the allergen chemically so that its ability to cause an immune response in the patient is unchanged, while its ability to cause an allergic reaction is substantially altered.

These methods, however, can take several years to be effective and are associated with the risk of side effects such as anaphylactic shock. The use of an imidazoquinoline agent and asthma/allergy medicament in combination with an allergen avoids many of the side effects etc. Other asthma/allegy medicaments that can be used in the methods and compositions of the invention are listed in U.S. Non-Provisional patent application Ser. No. 09/776,479, filed Feb. 2, 2001.

Imidazoquinoline agents can be combined with still other therapeutic agents such as adjuvants to enhance immune responses. The imidazoquinoline agent and other therapeutic agent may be administered simultaneously or sequentially. When the other therapeutic agents are administered simultaneously they can be administered in the same or separate formulations, but are administered at the same time. The other therapeutic agents are administered sequentially with one another and with imidazoquinoline agents, when the administration of the other therapeutic agents and the imidazoquinoline agent is temporally separated. The separation in time between the administration of these compounds may be a matter of minutes or it may be longer. Other therapeutic agents include but are not limited to adjuvants, cytokines, antibodies, antigens, etc.

The imidazoquinoline agents are useful as adjuvants for inducing a systemic immune response. Thus either can be delivered to a subject exposed to an antigen to produce an enhanced immune response to the antigen.

In addition to the imidazoquinoline agents, the compositions of the invention may also be administered with non-nucleic acid adjuvants. A non-nucleic acid adjuvant is any molecule or compound except for the imidazoquinoline agents described herein which can stimulate the humoral and/or cellular immune response. Non-nucleic acid adjuvants include, for instance, adjuvants that create a depot effect, immune stimulating adjuvants, and adjuvants that create a depot effect and stimulate the immune system.

An adjuvant that creates a depot effect as used herein is an adjuvant that causes the antigen to be slowly released in the body, thus prolonging the exposure of immune cells to the antigen. This class of adjuvants includes but is not limited to alum (e.g., aluminum hydroxide, aluminum phosphate); emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.); poly-arginine or poly lysine.

An immune stimulating adjuvant is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21^{st} peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Adjuvants that create a depot effect and stimulate the immune system are those compounds which have both of the above- identified functions. This class of adjuvants includes but is not limited to ISCOMS (Immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The imidazoquinoline agents are also useful as mucosal adjuvants.

Other mucosal adjuvants (including nucleic and non-nucleic acid mucosal adjuvants) may also be administered with the imidazoquinoline agents. A non-nucleic acid mucosal adjuvant as used herein is an adjuvant other than a immunostimulatory nucleic acid that is capable of inducing a mucosal immune response in a subject when administered to a mucosal surface in conjunction with an antigen. Mucosal adjuvants include but are not limited to Bacterial toxins e.g., Cholera toxin (CT), CT derivatives including but not limited to CTB subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); and CTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler B D, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi, outer membrane protine of Neisseria meningitidis) (Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worcester, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micelle-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

Immune responses can also be induced or augmented by the co-administration or co-linear expression of cytokines (Bueler & Mulligan, 1996; Chow et al., 1997; Geissler et al., 1997; Iwasaki et al., 1997; Kim et al., 1997) or B-7 co-stimulatory molecules (Iwasaki et al., 1997; Tsuji et al., 1997) with the Imidazoquinoline agents. The cytokines can be administered directly with Imidazoquinoline agents or may be administered in the form of a nucleic acid vector that encodes the cytokine, such that the cytokine can be expressed in vivo. In one embodiment, the cytokine is administered in the form of a plasmid expression vector. The term cytokine is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), IFN-alpha, IFN-gamma, tumor necrosis factor (TNF), transforming growth factor beta (TGF-beta), FLT-3 ligand, and CD40 ligand.

The compositions and methods of the invention can be used to modulate an immune response. The ability to modulate an immune response allows for the prevention and/or treatment of particular disorders that can be affected via immune system modulation.

Treatment after a disorder has started aims to reduce, ameliorate or altogether eliminate the disorder, and/or its associated symptoms, or prevent it from becoming worse. Treatment of subjects before a disorder has started (i.e., prophylactic treatment) aims to reduce the risk of developing the disorder. As used herein, the term “prevent” refers to the prophylactic treatment of patients who are at risk of developing a disorder (resulting in a decrease in the probability that the subject will develop the disorder), and to the inhibition of further development of an already established disorder.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular imidazoquinoline agent or other therapeutic agent being administered (e.g., in the case of an immunostimulatory nucleic acid, the type of nucleic acid, i.e., a CpG nucleic acid, the number of unmethylated CpG motifs or their location in the nucleic acid, the degree of modification of the backbone to the oligonucleotide, etc.), the size of the subject, or the severity of the disease or condition. One of ordinary skill in the art can empirically determine the effective amount of a particular imidazoquinoline agent and/or other therapeutic agent without necessitating undue experimentation.

The term “effective amount” of an imidazoquinoline agent refers to the amount necessary or sufficient to realize a desired biologic effect. In general, an effective amount of an imidazoquinoline agent is that amount necessary to cause activation of the immune system, resulting potentially in the development of an antigen specific immune response. In some embodiments, the imidazoquinoline agent are administered in an effective amount to stimulate or induce a Th1 immune response or a general immune response. An effective amount to stimulate a Th1 immune response may be defined as that amount which stimulates the production of one or more Th1-type cytokines such as interleukin 2 (IL-2), IL-12, tumor necrosis factor (TNF-alpha) and interferon gamma (IFN-gamma), and/or production of one or more Th1-type antibodies.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

The imidazoquinoline agents vary greatly in their potency, so the dose that would be used in the methods described herein may vary over several orders of magnitude and will probably be dependent upon the other therapeutic agent used and the therapeutic benefit desired. As an example, the previously described compound S-28463 (Tomai et al., Antiviral Res. 28:253, 1995) will be effective at inducing ADCC in a human subject when administered at doses between approximately 0.1 to 1.0 mg/kg. Since S-28463 (Resiquimod) is an enhanced version of Imiquimod, other agent within this class could be less potent for immunostimulation, but nevertheless still useful and possibly more useful as therapeutic agents. Alternatively, other imidazoquinoline agents may be several orders of magnitude more potent than S-28463.

Doses of the compounds described herein for parenteral delivery for the purpose of inducing an innate immune response or for increasing ADCC or for inducing an antigen specific immune response when the imidazoquinoline agents are administered in combination with other therapeutic agents or in specialized delivery vehicles typically range from about 0.1 μg to 10 mg per administration, which depending on the application could be given daily, weekly, or monthly and any other amount of time therebetween. More typically parenteral doses for these purposes range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

According to some aspects of the invention, an effective amount is that amount of an imidazoquinoline agent and that amount of another therapeutic agent, such as an antibody, an antigen, an immunostimulatory nucleic acid or a disorder-specific medicament which when combined or co-administered, results in a synergistic response. A synergistic amount is that amount which produces a response that is greater than the sum of the individual effects of the imidazoquinoline agent and the other therapeutic(s) alone.

As an example, a synergistic combination of an imidazoquinoline agent and a cancer medicament provides a biological effect which is greater than the combined biological effect which could have been achieved using each of the components (i.e., the agent and the medicament) separately. The biological effect may be the amelioration and or absolute elimination of symptoms resulting from the cancer. In another embodiment, the biological effect is the complete abrogation of the cancer, as evidenced for example, by the absence of a tumor or a biopsy or blood smear which is free of cancer cells.

As another example, an effective amount of an imidazoquinoline agent and an asthma/allergy medicament is that amount necessary to prevent the development of IgE, or to cause a reduction in IgE levels, or to cause the shift to a Th1 response, in response to an allergen or initiator. In other embodiments, the physiological result is a shift from Th2 cytokines, such as IL-4 and IL-5, to Th1 cytokines, such as IFN-gamma and IL-12.

In order to determine the effective amount of imidazoquinoline agent can be determined using in vitro stimulation assays. The stimulation index of the imidazoquinoline agent can be compared to that of previously tested immunostimulatory acids. The stimulation index can be used to determine an effective amount of the particular imidazoquinoline agent for the particular subject, and the dosage can be adjusted upwards or downwards to achieve the desired levels in the subject. Effective amounts of imidazoquinoline agents can also be determined from animal models, or from human clinical trials using imidazoquinoline agents and for compounds which are known to exhibit similar pharmacological activities, such as immunostimulatory nucleic acids and adjuvants, e.g., LT and other antigens for vaccination purposes.

In some instances, a sub-therapeutic dosage of either the imidazoquinoline agent or the other therapeutic agent, or a sub-therapeutic dosage of both, is used in the treatment of a subject having, or at risk of developing, a disorder. As an example, it has been discovered according to the invention, that when the two classes of drugs are used together, the medicament can be administered in a sub-therapeutic dose and still produce a desirable therapeutic result. A “sub-therapeutic dose” as used herein refers to a dosage which is less than that dosage which would produce a therapeutic result in the subject if administered in the absence of the other agent. Therapeutic doses of certain medicaments are well known in the field of medicine and these dosages have been extensively described in references such as Remington's Pharmaceutical Sciences, 18th ed., 1990; as well as many other medical references relied upon by the medical profession as guidance. Therapeutic dosages of imidazoquinoline agents have also been described in the art and methods for identifying therapeutic dosages in subjects are described in more detail herein.

In other aspects, the method of the invention involves administering a high dose of a disorder-specific medicament to a subject, without inducing side effects. Ordinarily, when a medicament is administered in a high dose, a variety of side effects can occur, as discussed in more detail above, as well as in the medical literature. As a result of these side effects, the medicament is not administered in such high doses, no matter what therapeutic benefits are derived. It was discovered, according to the invention, that such high doses of medicaments which ordinarily induce side effects can be administered without inducing the side effects as long as the subject also receives an imidazoquinoline agent. The type and extent of the side effects ordinarily induced by the medicament will depend on the particular medicament used.

Administration of the imidazoquinoline agent can occur prior to, concurrently with, or following administration of the antibody. If the imidazoquinoline agent is administered prior to the antibody, typically there is a 1 to 7 day interval between the administrations. If the imidazoquinoline agent is administered following the antibody, typically there is a 2-3 day interval between the administrations.

In embodiments of the invention in which the imidazoquinoline agent is administered on a routine schedule. The other therapeutic agents including antibodies, antigens, immunostimulatory nucleic acids and disorder-specific medicaments may also be administered on a routine schedule, but alternatively, may be administered as symptoms arise.

A “routine schedule” as used herein, refers to a predetermined designated period of time. The routine schedule may encompass periods of time which are identical or which differ in length, as long as the schedule is predetermined. For instance, the routine schedule may involve administration on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration on a daily basis for the first week, followed by a monthly basis for several months, and then every three months after that. Any particular combination would be covered by the routine schedule as long as it is determined ahead of time that the appropriate schedule involves administration on a certain day.

In methods particularly directed at subjects at risk of developing a disorder, timing of the administration of the imidazoquinoline agent and the disorder-specific medicament may also be particularly important. For instance, in a subject with a genetic predisposition to cancer, the imidazoquinoline agent and the cancer medicament, preferably in the form of an immunotherapy or a cancer medicament, may be administered to the subject on a regular basis.

In some aspects of the invention, the imidazoquinoline agent is administered to the subject in anticipation of an asthmatic or allergic event in order to prevent an asthmatic or allergic event. The asthmatic or allergic event may be, but need not be limited to, an asthma attack, seasonal allergic rhinitis (e.g., hay-fever, pollen, ragweed hypersensitivity) or perennial allergic rhinitis (e.g., hypersensitivity to allergens such as those described herein). In some instances, the imidazoquinoline agent is administered substantially prior to an asthmatic or an allergic event. As used herein, “substantially prior” means at least six months, at least five months, at least four months, at least three months, at least two months, at least one month, at least three weeks, at least two weeks, at least one week, at least 5 days, or at least 2 days prior to the asthmatic or allergic event.

Similarly, the asthma/allergy medicament may be administered immediately prior to the asthmatic or allergic event (e.g., within 48 hours, within 24 hours, within 12 hours, within 6 hours, within 4 hours, within 3 hours, within 2 hours, within 1 hour, within 30 minutes or within 10 minutes of an asthmatic or allergic event), substantially simultaneously with the asthmatic or allergic event (e.g., during the time the subject is in contact with the allergen or is experiencing the asthma or allergy symptoms) or following the asthmatic or allergic event.

The compositions of the invention may be delivered to a particular tissue or cell type or to the immune system or both. In its broadest sense, a “vector” is any vehicle capable of facilitating the transfer of the compositions to the target cells. The vector generally transports the imidazoquinoline agent, antibody, antigen, immunostimulatory nucleic acid and/or disorder-specific medicament to the target cells with reduced degradation relative to the extent of degradation that would result in the absence of the vector.

In general, the vectors useful in the invention are divided into two classes: biological vectors and chemical/physical vectors. Biological vectors and chemical/physical vectors are useful in the delivery and/or uptake of therapeutic agents of the invention.

Most biological vectors are used for delivery of nucleic acids and this would be most appropriate in the delivery of imidazoquinoline agents and targeting agents that are immunostimulatory nucleic acids.

In addition to the biological vectors discussed herein, chemical/physical vectors may be used to deliver imidazoquinoline agents and targeting agents, antibodies, antigens, and disorder specific medicaments. As used herein, a “chemical/physical vector” refers to a natural or synthetic molecule, other than those derived from bacteriological or viral sources, capable of delivering the nucleic acid and/or a cancer medicament.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA and intact virions can be encapsulated within the aqueous interior and be delivered to cells in a biologically active form (Fraley, et al., Trends Biochem. Sci., (1981) 6:77).

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to the cancer by coupling it to a one of the immunotherapeutic antibodies discussed earlier. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2,3 dioleyloxy)-propyl]-N, N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one embodiment, the vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System”. PCT/US/0307 describes a biocompatible, preferably biodegradable polymeric matrix for containing an exogenous gene under the control of an appropriate promoter. The polymeric matrix can be used to achieve sustained release of the imidazoquinoline agent and/or the cancer medicament in the subject.

The polymeric matrix preferably is in the form of a microparticle such as a microsphere (wherein the imidazoquinoline agent and/or the other therapeutic agent is dispersed throughout a solid polymeric matrix) or a microcapsule (wherein the imidazoquinoline agent and/or the other therapeutic agent is stored in the core of a polymeric shell). Other forms of the polymeric matrix for containing the imidazoquinoline agent and/or the other therapeutic agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid and/or the other therapeutic agent are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the imidazoquinoline agents are administered to the subject via an implant while the other therapeutic agent is administered acutely. Biocompatible microspheres that are suitable for delivery, such as oral or mucosal delivery are disclosed in Chickering et al., Biotech. And Bioeng., (1996) 52:96-101 and Mathiowitz et al., Nature, (1997) 386:.410-414 and PCT Patent Application WO97/03702.

Both non-biodegradable and biodegradable polymeric matrices can be used to deliver the imidazoquinoline agent and/or other therapeutic agent to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable, particularly for the imidazoquinoline agents. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

If the therapeutic agent is a nucleic acid, the use of compaction agents may also be desirable. Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake of a nucleic acid include calcium phosphate and other chemical mediators of intracellular transport, microinjection compositions, electroporation and homologous recombination compositions (e.g., for integrating a nucleic acid into a preselected location within the target cell chromosome).

The compounds may be administered alone (e.g. in saline or buffer) or using any delivery vectors known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The imidazoquinoline agents useful in the invention may be delivered in mixtures with additional adjuvant(s), other therapeutics, or antigen(s). A mixture may consist of several adjuvants in addition to the imidazoquinoline agent or several antigens or other therapeutics.

The imidazoquinoline agents and other compounds can be administered by any ordinary route for administering medications. A variety of administration routes are available. The particular mode selected will depend, of course, upon the particular adjuvants or antigen selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed herein. For use in therapy, an effective amount of the imidazoquinoline agent can be administered to a subject by any mode that delivers the agent to the desired surface, e.g., mucosal, systemic.

Administering the pharmaceutical composition of the present invention may be accomplished by any means known to the skilled artisan. Preferred routes of administration include but are not limited to oral, parenteral, intramuscular, intranasal, intratracheal, inhalation, ocular, vaginal, and rectal. For the treatment or prevention of asthma or allergy, such compounds are preferably inhaled, ingested or administered by systemic routes. Systemic routes include oral and parenteral. Inhaled medications are preferred in some embodiments because of the direct delivery to the lung, the site of inflammation, primarily in asthmatic patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

For oral administration, the compounds (i.e., imidazoquinoline agents, antigens, antibodies, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The imidazoquinoline agents and optionally other therapeutics and/or antigens may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories. For treatment of a patient, depending on activity of the compound, manner of administration, purpose of the immunization (i.e., prophylactic or therapeutic), nature and severity of the disorder, age and body weight of the patient, different doses may be necessary. The administration of a given dose can be carried out both by single administration in the form of an individual dose unit or else several smaller dose units. Multiple administration of doses at specific intervals of weeks or months apart is usual for boosting the antigen-specific responses.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In other aspects of the invention, a composition is provided. The composition includes an imidazoquinoline agent and another therapeutic agent formulated in a pharmaceutically-acceptable carrier and present in the composition in an effective amount.

In other aspects, the invention relates to kits. One kit of the invention includes a sustained release vehicle containing an imidazoquinoline agent and a container housing another therapeutic agent and instructions for timing of administration of the compounds. A sustained release vehicle is used herein in accordance with its prior art meaning of any device which slowly releases the compound contained therein.

The container may be a single container housing all of a medicament together or it may be multiple containers or chambers housing individual dosages of the medicament, such as a blister pack. The kit also has instructions for timing of administration of the medicament. The instructions would direct the subject to take the medicament at the appropriate time. For instance, the appropriate time for delivery of the medicament may be as the symptoms occur. Alternatively, the appropriate time for administration of the medicament may be on a routine schedule such as monthly or yearly.

Another kit of the invention includes at least one container housing an imidazoquinoline agent and at least one container housing another therapeutic agent and instructions for administering the compositions in effective amounts for inducing a synergistic immune response in the subject. The instructions in the kit may direct the subject to take compounds in amounts which will produce a synergistic immune response. The drugs may be administered simultaneously or separately as long as they are administered close enough in time to produce a synergistic response.

R-848 (Resiquimod) and R-847 (Imiquimod) belong to the family of imidazoquinolines, a class of immune response modifiers shown to possess antiviral and antitumor activities. Imiquimod is already clinically approved for treatment of human papilloma virus (HPV)-related genital warts. R-848 and R-847 are potent inducers of cytokines, including IFN-alpha, IL-12 and IFN-gamma. Like the CpG ODN 2006, they enhance Th1-mediated immune responses while inhibiting Th2 responses. Both R-848 and CpG ODN activate macrophages and DCs to secrete many of the same cytokines. However, R-848 and CpG ODN induce nearly the same cytokines with different kinetics and relative amounts as shown in studies in mice. Vasilakos JP et al. (2000) Cell Immunol 204:64-74. The present inventors have now shown that R-848 induces substantially more of the proinflammatory cytokines TNF-alpha and IL-6 in PBMC than CpG ODN 2006.

Mechanisms of R-848 activation and CpG ODN activation appear to be different. While chloroquine can completely abolish the effects of CpG ODN 2006, chloroquine appears to be able to dampen but not abolish R-848-mediated signaling. It has been shown that CpG ODN 2006 activates two cell types directly, B-cells and pDCs. Krug A et al. (2001) unpublished observations. Studies of Ahonen et al. revealed that R-848 can activate mDCs directly. Ahonen CL et al. (1999) Cell Immunol 197:62-72.

Although both R-848 and CpG-ODN stimulate NF-kappa B activation, the mechanism of activation appears to be different. CpG-ODN activate Toll-like receptor 9 (hTLR9). Hemmi H et al. (2000) Nature 408:740-5; Bauer S et al. (2001) Proc Natl Acad Sci USA 98:9237-42. TLR9 belongs to a family of immune receptors which function as mediators of innate immunity for recognition of pathogen-derived ligands. To date, there are ten TLR proteins known. The ligands of some, but not all, the various TLRs are also characterized. For example, lipopolysaccharide (LPS), a component of gram-negative bacteria, is recognized by TLR4. Chow J C et al. (1999) J Biol Chem 274:10689-92. Expression patterns of all known TLR proteins is complex. While hTLR1 is ubiquitously expressed, hTLR2, hTLR4 and hTLR5 are present in monocytes, polymorphonuclear phagocytes and dendritic cells. Muzio M et al. (2000) J Leukoc Biol 67:450-6. Research done in the group of G. Hartmann (Krug A et al. (2001); Homung V et al. (2001), both unpublished observations) showed that hTLR7 and hTLR9 are present in B cells and pDCs, while mDCs express hTLR7 and hTLR8 but not hTLR9. Human TLR8, however, appears not to be expressed in pDCs.

According to one aspect of the instant invention, applicants have discovered that R-848-mediated NF-kappa B activation in human embryo kidney cells is mediated through a member of the human Toll-like receptor family, hTLR8. 293T cells transiently transfected with a hTLR8 cDNA expression vector activated NF-kappa B signaling in response to R-848, but not CpG-ODN. Activation through hTLR8 was observed to vary with R-848 in a dose-dependent manner.

Applicants also observed activation of NF-kappa B signaling when 293T cells transiently transfected with a hTLR7 cDNA expression vector were contacted with R-848. In contrast to the situation with TLR8, the activation through TLR7 was observed to be concentration-independent, suggesting that (1) hTLR7 might be even more sensitive to R-848 than hTLR8, and (2) the concentrations examined were enough to saturate hTLR7 signaling. While NF-kappa B activation by CpG ODN 2006 is mediated through hTLR9, R-848 appeared not to activate any NF-kappa B signaling in cells expressing hTLR9 alone. 293T cells expressing hTLR8 also produced IL-8 in response to R-848.

The identification by the applicants of TLR8 and TLR7 as receptors for the imidazoquinoline R-848 forms part of the basis for the screening methods described herein. The screening methods of the present invention take advantage of the fact that binding of imidazoquinoline by TLR8 or TLR7 gives rise to TLR-mediated signaling activity. The TLR7 or TLR8 signaling activity of an imidazoquinoline can be used as a reference response against which TLR signaling activity of test compound can be compared in various screening assays described herein.

A basis for certain of the screening assays is the presence of a functional TLR, e.g., TLR7, TLR8, or TLR9. A functional TLR is a full-length TLR polypeptide or a fragment thereof capable of inducing a signal in response to interaction with a TLR ligand. For example, TLR4 and other TLRs have a cytoplasmic Toll/IL-1 receptor (TIR) homology domain. This domain communicates with a similar domain on an adapter protein (MyD88) that interacts with TLR4 by means of a like:like interaction of TIR domains. The next interaction is between the adapter and a kinase, through their respective “death domains.” The kinase in turn interacts with tumor necrosis factor (TNF) receptor-associated factor-6 (TRAF6). Medzhitov R et al., Mol Cell 2:253 (1998); Kopp EB et al., Curr Opin Immunol 11:15 (1999). After TRAF6, two sequential kinase activation steps lead to phosphorylation of the inhibitory protein I kappa B and its dissociation from NF-kappa B. The first kinase is a mitogen-activated kinase kinase kinase (MAPKKK) known as NIK, for NF-kappa B-inducing kinase. The target of this kinase is another kinase made up of two chains, called I kappa B kinase alpha (IKK alpha) and I kappa B kinase beta (IKK beta), that together form a heterodimer of IKKalpha:IKKbeta, which phosphorylates I kappa B. NF-kappa B translocates to the nucleus to activate genes with kappa B binding sites in their promoters and enhancers such as the genes encoding interleukin-1 beta (IL-1 beta), IL-6, IL-8, the p40 subunit of IL-12, and the costimulatory molecules CD80 and CD86.

The functional TLR in some instances is naturally expressed by a cell. In other instances, expression of the functional TLR can involve introduction or reconstitution of a species-specific TLR into a cell or cell line that otherwise lacks the TLR or lacks responsiveness to a recognized ligand of the TLR, resulting in a cell or cell line capable of activating the TLR/IL-1R signaling pathway in response to contact with a suitable ligand. Examples of cell lines lacking TLR9 or immunostimulatory nucleic acid responsiveness include, but are not limited to, 293 fibroblasts (ATCC CRL-1573), MonoMac-6, THP-1, U937, CHO, and any TLR9 knock-out. The introduction of the species-specific TLR into the cell or cell line is preferably accomplished by transient or stable transfection of the cell or cell line with a TLR-encoding nucleic acid sequence operatively linked to a gene expression sequence.

The functional TLR, including TLR7, TLR8, and TLR9, is not limited to a human TLR, but rather can include a TLR derived from human or non-human sources. Examples of non-human sources include, but are not limited to, murine, bovine, canine, feline, ovine, porcine, and equine. Other species include chicken and fish, e.g., aquaculture species.

The functional TLR, including TLR7, TLR8, and TLR9, also is not limited to native TLR polypeptides. In certain embodiments the TLR can be, e.g., a chimeric TLR in which the extracellular domain and the cytoplasmic domains are derived from TLR polypeptides from different species. Such chimeric TLR polypeptides, as described above, can include, for example, a human TLR extracellular domain and a murine TLR cytoplasmic domain, each domain derived from the corresponding TLR7, TLR8, or TLR9 of each species. In alternative embodiments, such chimeric TLR polypeptides can include chimeras created with different TLR splice variants or allotypes. Other chimeric TLR polypeptides useful for the purposes of screening ISNA mimics, agonists and antagonists can include chimeric polypeptides created with a TLR of a first type, e.g., TLR9, and another TLR, e.g., TLR7 or TLR8, of the same or another species as the TLR of the first type. Also contemplated are chimeric polypeptides which incorporate sequences derived from more than two polypeptides, e.g., an extracellular domain, a transmembrane domain, and a cytoplasmic domain all derived from different polypeptide sources, provided at least one such domain derives from a TLR7, TLR8, or TLR9 polypeptide. As a further example, also contemplated are constructs such as include an extracellular domain of one TLR9, an intracellular domain of another TLR9, and a non-TLR reporter such as luciferase, GFP, etc. Those of skill in the art will recognize how to design and generate DNA sequences coding for such chimeric TLR polypeptides.

The screening assays can have any of a number of possible readout systems based upon either TLR/IL-1R signaling pathway or other assays suitable for assaying TLR signaling activity. In preferred embodiments, the readout for the screening assay is based on the use of native genes or, alternatively, cotransfected or otherwise co-introduced reporter gene constructs which are responsive to the TLR/IL-1R signal transduction pathway involving MyD88, TRAF6, p38, and/or ERK. Häcker H et al., EMBO J 18:6973-6982 (1999). These pathways activate kinases including kappa B kinase complex and c-Jun N-terminal kinases. Thus reporter genes and reporter gene constructs particularly useful for the assays can include a reporter gene operatively linked to a promoter sensitive to NF-kappa B. Examples of such promoters include, without limitation, those for NF-kappa B, IL-1beta, IL-6, IL-8, IL-12 p40, CD80, CD86, and TNF-alpha. The reporter gene operatively linked to the TLR7-, TLR8-, or TLR9-sensitive promoter can include, without limitation, an enzyme (e.g., luciferase, alkaline phosphatase, beta-galactosidase, chloramphenicol acetyltransferase (CAT), etc.), a bioluminescence marker (e.g., green-fluorescent protein (GFP, U.S. Pat. No. 5,491,084), etc.), a surface-expressed molecule (e.g., CD25), and a secreted molecule (e.g., IL-8, IL-12 p40, TNF-alpha). In preferred embodiments the reporter is selected from IL-8, TNF-alpha, NF-kappa B-luciferase (NF-kappa B-luc; Häcker H et al., EMBO J 18:6973-6982 (1999)), IL-12 p40-luc (Murphy TL et al., Mol Cell Biol 15:5258-5267 (1995)), and TNF-luc (Häcker H et al., EMBO J 18:6973-6982 (1999)). In assays relying on enzyme activity readout, substrate can be supplied as part of the assay, and detection can involve measurement of chemiluminescence, fluorescence, color development, incorporation of radioactive label, drug resistance, or other marker of enzyme activity. For assays relying on surface expression of a molecule, detection can be accomplished using FACS analysis or functional assays. Secreted molecules can be assayed using enzyme-linked immunosorbent assay (ELISA) or bioassays. Many such readout systems are well known in the art and are commercially available.

As mentioned above, the invention in one aspect provides a screening method for comparing TLR signaling activity or a test compound against corresponding TLR signaling activity of a reference imidazoquinoline. The methods generally involve contacting a functional TLR selected from the group consisting of TLR7 and TLR8 with a reference imidazoquinoline and detecting a reference response mediated by a TLR signal transduction pathway; contacting a functional TLR selected from the group consisting of TLR7 and TLR8 with a test compound and detecting a test response mediated by a TLR signal transduction pathway; and comparing the test response with the reference response to compare the TLR signaling activity of the test compound with the imidazoquinoline. Assays in which the test compound and the reference imidazoquinoline contact the TLR independently may be used to identify test compounds that are imidazoquinoline mimics. Assays in which the test compound and the reference imidazoquinoline contact the TLR concurrently may be used to identify test compounds that are imidazoquinoline agonists and imidazoquinoline antagonists.

An imidazoquinoline mimic as used herein is a compound which causes a response mediated by a TLR signal transduction pathway. As used herein the term “response mediated by a TLR signal transduction pathway” refers to a response which is characteristic of an imidazoquinoline-TLR interaction. As demonstrated herein responses which are characteristic of imidazoquinoline-TLR interactions include the induction of a gene under control of an imidazoquinoline-specific promoter such as a NF-kappa B promoter, increases in Th1 cytokine levels, etc. The gene under the control of the NF-kappa B promoter may be a gene which naturally includes an NF-kappa B promoter or it may be a gene in a construct in which an NF-kappa B promoter has been inserted. Genes which naturally include the NF-kappa B promoter include but are not limited to IL-8, IL-12 p40, NF-kappa B-luc, IL-12 p40-luc, and TNF-luc. Increases in Th1 cytokine levels is another measure characteristic of an imidazoquinoline-TLR interaction. Increases in Th1 cytokine levels may result from increased production or increased stability or increased secretion of the Th1 cytokines in response to the imidazoquinoline-TLR interaction. Th1 cytokines include but are not limited to IL-2, IFN-alpha, and IL-12. Other responses which are characteristic of an imidazoquinoline-TLR interaction include but are not limited to a reduction in Th2 cytokine levels. Th2 cytokines include but are not limited to IL-4, IL-5, IL-10, and IL-13.

The response which is characteristic of an imidazoquinoline-TLR interaction may be a direct response or an indirect response. A direct response is a response that arises directly as a result of the imidazoquinoline-TLR interaction. An indirect response is a response which involves the modulation of other parameters prior to its occurrence.

An imidazoquinoline agonist as used herein is a compound which causes an enhanced response to an imidazoquinoline mediated by a TLR signal transduction pathway. Thus an imidazoquinoline agonist as used herein is a compound which causes an increase in at least one aspect of an immune response that is ordinarily induced by the reference imidazoquinoline. For example, an immune response that is ordinarily induced by an imidazoquinoline can specifically include TLR7- or TLR8-mediated signal transduction in response to an imidazoquinoline. An imidazoquinoline agonist will in some embodiments compete with imidazoquinoline for binding to TLR7 or TLR8. In other embodiments an imidazoquinoline agonist will bind to a site on TLR7 or TLR8 that is distinct from the site for binding imidazoquinoline. In yet other embodiments an imidazoquinoline agonist will act via another molecule or pathway distinct from TLR7 or TLR8.

An imidazoquinoline antagonist as used herein is a compound which causes a decreased response to an imidazoquinoline mediated by a TLR signal transduction pathway. Thus an imidazoquinoline antagonist as used herein is a compound which causes a decrease in at least one aspect of an immune response that is ordinarily induced by the reference imidazoquinoline. For example, an immune response that is ordinarily induced by an imidazoquinoline can specifically include TLR7- or TLR8-mediated signal transduction in response to an imidazoquinoline. An imidazoquinoline antagonist will in some embodiments compete with imidazoquinoline for binding to TLR7 or TLR8. In other embodiments an imidazoquinoline antagonist will bind to a site on TLR7 or TLR8 that is distinct from the site for binding imidazoquinoline. In yet other embodiments an imidazoquinoline antagonist will act via another molecule or pathway distinct from TLR7 or TLR8.

The screening methods for comparing TLR signaling activity of a test compound with signaling activity of an imidazoquinoline involve contacting at least one test compound with a functional TLR selected from TLR7 and TLR8 under conditions which, in the absence of a test compound, permit a reference imidazoquinoline to induce at least one aspect of an immune response. The functional TLR may be expressed by a cell or it may be part of a cell-free system. A cell expressing a functional TLR is a cell that either naturally expresses the TLR, or is a cell into which has been introduced a TLR expression vector, or is a cell manipulated to express TLR in a manner that allows the TLR to be expressed by the cell and to transduce a signal under conditions which normally permit signal transduction by the signal transducing portion of the TLR. The TLR can be a native TLR or it can be a fragment or variant thereof, as described above. According to these methods, the test compound is contacted with a functional TLR or TLR-expressing cell before, after, or simultaneously with contacting a reference imidazoquinoline with the functional TLR or TLR-expressing cell. A response of the functional TLR or TLR-expressing cell is measured and compared with the corresponding response that results or would result under the same conditions in the absence of the test compound. Where it is appropriate, the response in the absence of the test compound can be determined as a concurrent or historical control. Examples of such responses include, without limitation, a response mediated through the TLR signal transduction pathway, secretion of a cytokine, cell proliferation, and cell activation. In a preferred embodiment, the measurement of a response involves the detection of IL-8 secretion (e.g., by ELISA). In another preferred embodiment, the measurement of the response involves the detection of luciferase activity (e.g., NF-kappa B-luc, IL-12 p40-luc, or TNF-luc).

Test compounds can include but are not limited to peptide nucleic acids (PNAs), antibodies, polypeptides, carbohydrates, lipids, hormones, and small molecules including, in particular, imidazoquinolines other than R-484 and R-487. Test compounds can further include variants of a reference imidazoquinoline. Test compounds can be generated as members of a combinatorial library of compounds.

In preferred embodiments, the methods for screening test compounds, test nucleic acid molecules, test imidazoquinolines, and candidate pharmacological agents can be performed on a large scale and with high throughput by incorporating, e.g., an array-based assay system and at least one automated or semi-automated step. For example, the assays can be set up using multiple-well plates in which cells are dispensed in individual wells and reagents are added in a systematic manner using a multiwell delivery device suited to the geometry of the multiwell plate. Manual and robotic multiwell delivery devices suitable for use in a high throughput screening assay are well known by those skilled in the art. Each well or array element can be mapped in a one-to-one manner to a particular test condition, such as the test compound. Readouts can also be performed in this multiwell array, preferably using a multiwell plate reader device or the like. Examples of such devices are well known in the art and are available through commercial sources. Sample and reagent handling can be automated to further enhance the throughput capacity of the screening assay, such that dozens, hundreds, thousands, or even millions of parallel assays can be performed in a day or in a week. Fully robotic systems are known in the art for applications such as generation and analysis of combinatorial libraries of synthetic compounds. See, for example, U.S. Pat. Nos. 5,443,791 and 5,708,158.

### EXAMPLES

Methods

Except where otherwise indicated, the following general methods were used.

Cells used for transfections were 293T (human embryo kidney cells, T-antigen transfected) or 293-TLR9-Luc (stable transfectants, human embryo kidney cells expressing the human TLR9 receptor and containing a genomic NF-kappa B-luciferase cassette).

Transfections were performed in six-well plates. Cells were plated the day before transfection at 4×10⁵/well in DMEM+10% FCS. Transfection was performed using cationic lipids (EFFECTENE™ reagent, QIAGEN) according to manufacturer's suggestion using 1 μg of DNA and 10 μl EFFECTENE™ per well.

Constructs: TLR cDNAs were cloned into pcDNA3.1. NF-kappa B activation was measured by using an 5×NF-kappa B-Luciferase construct (Stratagene). Transfection efficiency was determined by using a beta-galactosidase (beta-gal) reporter construct (p beta-Gal-Control, Clontech).

Stimulation was performed 24 h after transfection. Medium of the cells was reduced to 1 ml (without medium change) and cells were stimulated with indicated amounts of R-848, LPS, ODN 8954, 2006 and IL-1beta for 16 h.

Cell extracts were prepared by lysing the cells in 100 μl reporter lysis buffer using the freeze-thaw method. NF-kappa B stimulation was measured through luciferase activity (Promega). All data were normalized for beta-gal expression. Stimulation indices were calculated in reference to luciferase activity of medium without addition of ODN.

### Example 1

R-848 Does Not Stimulate hTLR9-Mediated NF-kappa B Activation

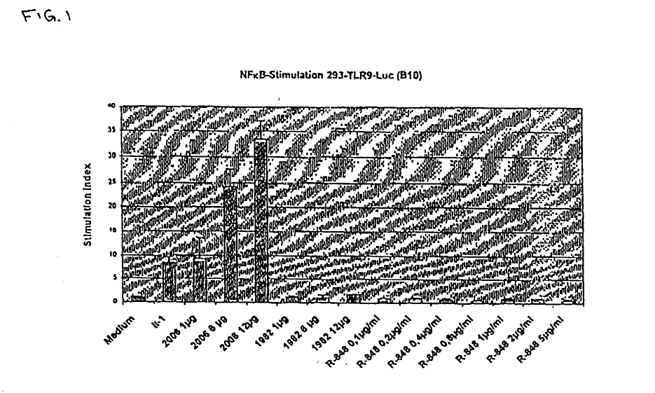

Since R-848 has immune modulatory properties, this experiment examined whether R-848-mediated immune responses are hTLR9-dependent. Cells stably transfected with hTLR9 and a NF-kappa B reporter construct (293-TLR-Luc cells) were incubated for 16 hours with IL-1, CpG ODN 2006, control non-CpG ODN 1982 (5′ TCCAGGACTTCTCTCAGGTT 3′, SEQ ID NO:3), or increasing amounts of R-848. NF-kappa B activation was determined by measurement of luciferase activity. Results are presented in FIG. 1. Activity is given in x-fold activation compared to luciferase activity in medium control. While CpG-ODN 2006 at concentrations ranging from 1 to 12 μg/ml stimulated NF-kappa B activation 10- to 30-fold, R-848 at 5 μg/ml did not yield any NF-kappa B activation.

### Example 2

Activation of NF-kappa B in 293T Cells by R-848 is Mediated through TLR8 and TLR7

293T cells, stably transfected with a NF-kappa B-luciferase reporter construct, were transiently transfected with plasmids (pcDNA3.1 constructs) coding for full length hTLR2, hTLR7, hTLR8 and hTLR9. All transfections were normalized to beta-galactosidase activity. Twenty-four hours following transfection, cells were stimulated with R-848, LPS, CpG ODN 8954 (5′ GGGGACGACGTCGTGGGGGGG 3′, SEQ ID NO:4), CpG ODN 2006, or IL-1 and then assayed for luciferase activity 16 h after stimulation. Each experiment was done at least twice with similar results.

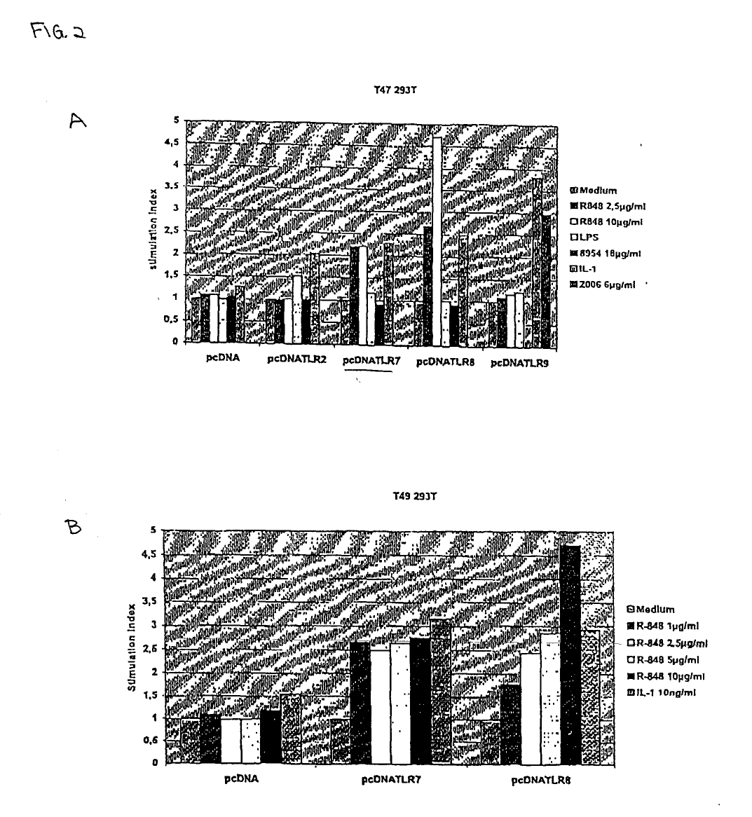

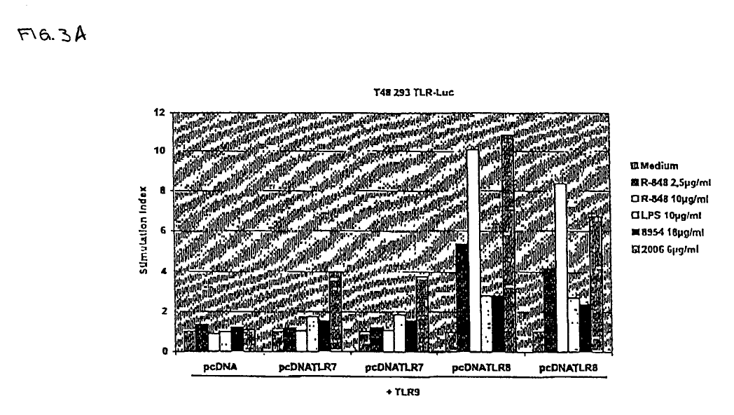

As shown in FIG. 2A, R-848 stimulated NF-kappa B-dependent transcription of the luciferase reporter gene 2.5- to 4.5-fold. The positive control IL-1activated the NF-kappa B luciferase reporter gene in a TLR-independent manner. Positive control for transfection of hTLR9 was addition of 2006, which stimulated NF-kappa B activation 3-fold. A response to R-848 was also seen in cells transfected with hTLR7. Neither LPS nor the CpG ODN 8954 appeared to activate hTLR7 or hTLR8. As a further control, hTLR2-transfected 293T cells were activated by LPS, consistent with earlier studies done by Chow et al. Chow J C et al. (1999) J Biol Chem 274:10689-92.

FIG. 2B shows hTLR8-mediated NF-kappa B activation varied in a dose-dependent manner with the concentration of R-848. Cells were stimulated 24 h after transfection and assayed 16 h later for luciferase activity. Increasing amounts (1 to 10 μg/ml) R-848 yielded an increase in stimulation ranging from 1.8- to 4.7-fold. In contrast, R-848-mediated hTLR7-activation did not appear to be concentration-dependent in this range, suggesting that hTLR7-signaling is saturated at the examined concentrations of R-848. The same results were obtained with R-848 which was purified by filtration.

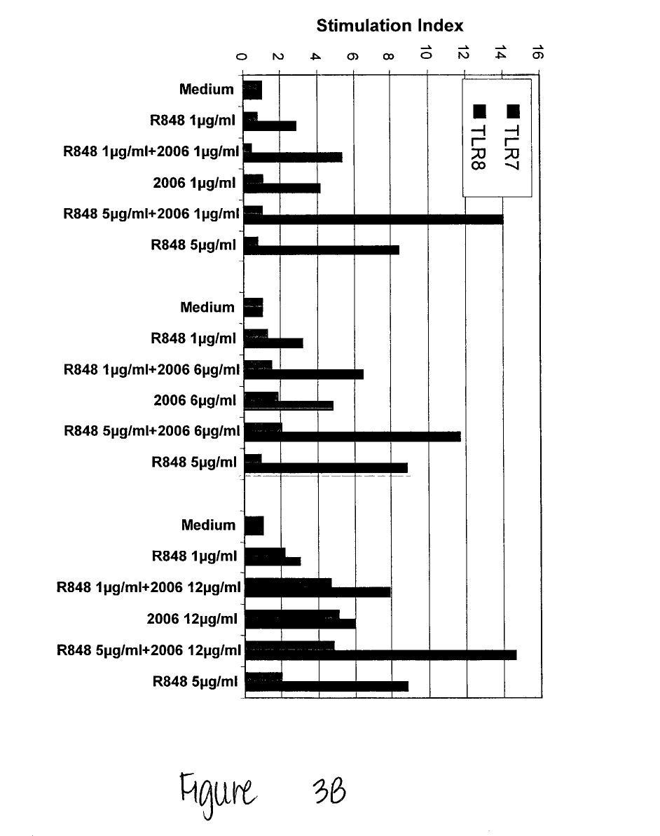

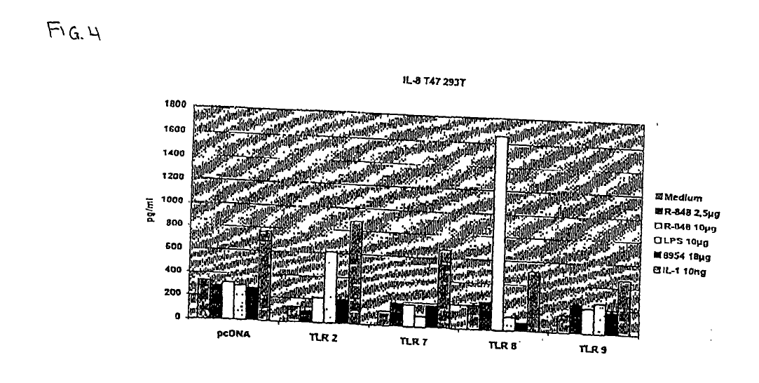

To confirm the role of hTLR8 in R-848-mediated activation, stably transfected 293-TLR9-Luc cells were transfected with hTLR-cDNA constructs and luciferase activity determined. 293-TLR9-Luc cells contain a genomic copy of the hTLR9 open reading frame and the NF-kappa B-luciferase cassette. Experiments were performed in duplicate, and CpG ODN 2006 was used as a positive control since the cells constitutively express TLR9. As shown in FIG. 3A, cells transfected with a construct expressing hTLR8 yielded in NF-kappa B activation in response to R-848. In contrast, cells transfected with empty vector or a construct expressing hTLR7 did not yield NF-kappa B activation in response to R-848 in this experiment.

Similar to the results of transient transfection experiments described above (FIG. 2), the observed response was R-848 concentration-dependent. Stimulation with 2.5 μg/ml R-848 resulted in a 5-fold increase in activation, while stimulation with 10 μg/ml R-848 resulted in a 10-fold increase of activity. Positive control in these experiments was stimulation with 6 μg/ml CpG ODN 2006 since the cells constitutively expressed hTLR9. In these experiments, hTLR7 unexpectedly appeared to be inactive upon R-848 stimulation. In all transfection experiments hTLR2 and hTLR6 were also examined, but neither of these showed any response to R-848. As already shown in FIG. 1, hTLR9 did not react to R-848 since transfection of 293-TLR-Luc with pcDNA (empty vector) alone did not result in any activation.

In still other experiments, the combined effect of R-848 and CpG ODN was tested on cells expressing TLR9 and either TLR7 or TLR8. (See FIG. 3B.) Co-stimulation was measured by NF-kappaB activation in 293-TLR9-Luc cells transfected with hTLR9 and hTLR7 (first bar of each pair), or hTLR9 and hTLR8 (second bar of each pair). As described above, activation in response to R-848 was concentration dependent in cells co-expressing hTLR8 and hTLR9. Cells expressing hLTR7 and hTLR9 were not activated in response to R-848. The addition of R-848 and CpG ODN #2006 resulted in activation levels greater than with either compound alone in hTLR8 but not hTLR7 expressing cells.

### Example 3

R-848 Induces IL-8 Production in the Presence of hTLR8

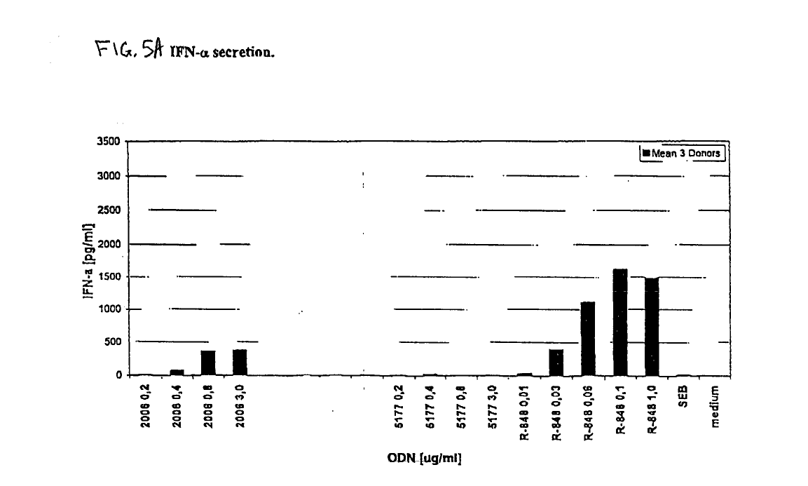

It is known that CpG ODN can induce IL-8 production in 293 cells transfected with hTLR9. Bauer S et al. (2001) Proc Natl Acad Sci USA 98:9237-42. The same was observed in this experiment in which 293T cells transfected with hTLR8 were stimulated with R-848. Cells were stimulated with R-848, LPS, ODN 8954, or IL-1 24 h after transfection. Supernatants were collected 16 h after stimulation, and the amount of IL-8 in the supernatants was determined by ELISA (OptELA, Becton-Dickinson). As shown in FIG. 4, stimulation of hTLR8-transfected 293T cells with 10 μg/ml R-848 resulted in greater than 1600 pg/ml IL-8 16 h after stimulation. Transfection with hTLR7 resulted in a slight increase of IL-8 production compared to background.

### Example 4

R-848 Induces IFN-alpha

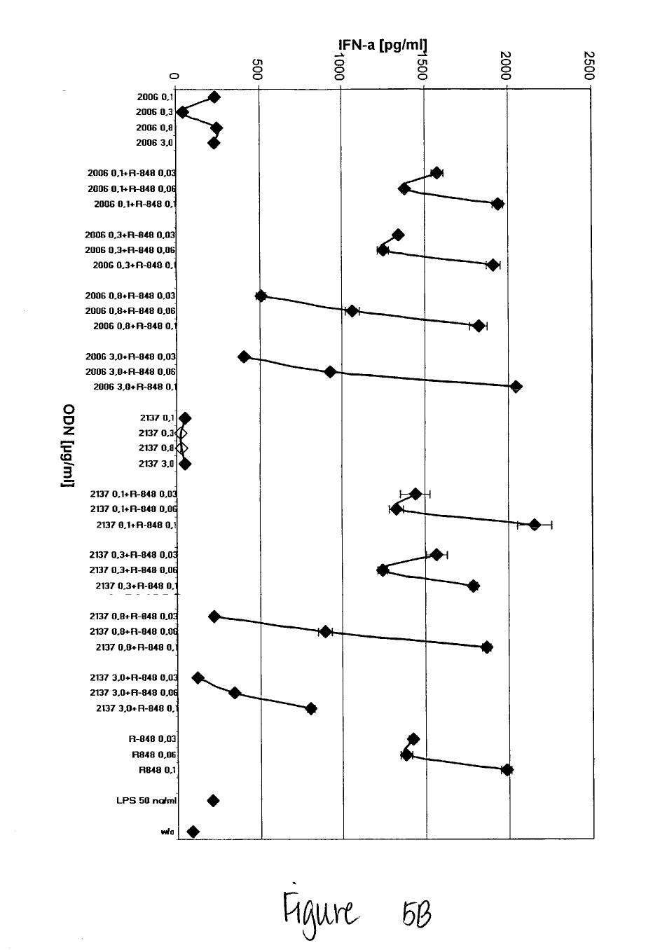

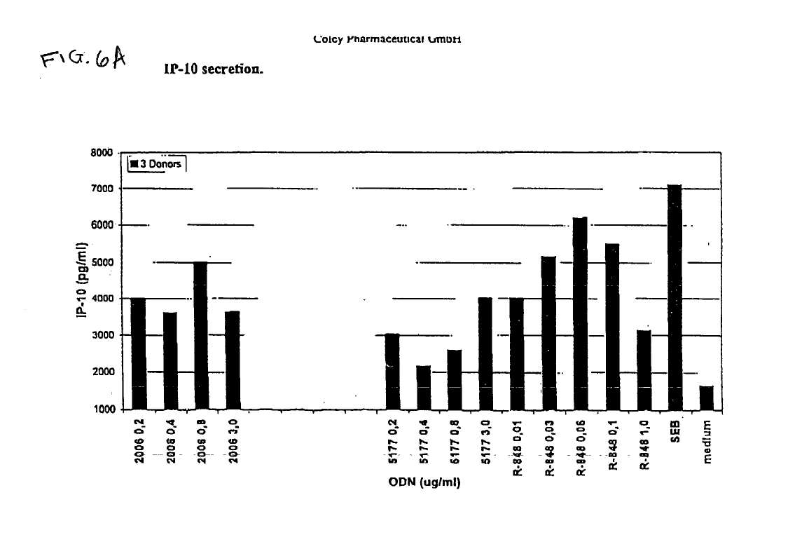

R-848 has been described to induce IFN-alpha in monocyte-derived dendritic cells (mDCs), whereas CpG ODNs have been described to induce the secretion of IFN-alpha from plasmacytoid dendritic cells (pDCs) (Krug A et al. (2001) Eur J Immunol 31:2154-63. In this experiment unfractionated human PBMC, containing mDCs and pDCs, were incubated for 48 hours in the presence of varying concentrations of R-848 (0.01-1.0 μg/ml), varying concentrations of CpG ODN 2006 (0.2-3.0 μg/ml), varying concentrations of negative control ODN 5177 (5′ TCCGCCTGTGACATGCATT 3′; SEQ ID NO:5; 0.2-3.0 μg/ml), Staphylococcal enterotoxin B (SEB, 50 ng/ml), or media alone, and then the concentration of IFN-alpha in the supernatant was measured by ELISA. R-848 induced higher amounts of IFN-alpha upon incubation of human PBMC than type B CpG ODN 2006 (FIG. 5A).

In FIG. 5B, the combined effects of R-848 and CpG ODN (e.g., #2006) on IFN-alpha secretion are shown. Human PBMCs from three different donors were incubated for 48 hours with the indicated concentrations of ODNs and R-848, either individually or together. Supernatants were harvested and IFN-alpha was measured by ELISA. The data represent mean cytokine amounts. The data suggest that a dose-dependent negative effect of IFN-alpha secretion results from the use of certain CpG ODNs together with R-848.

### Example 5

R-848 Induces IP-10 and IFN-gamma

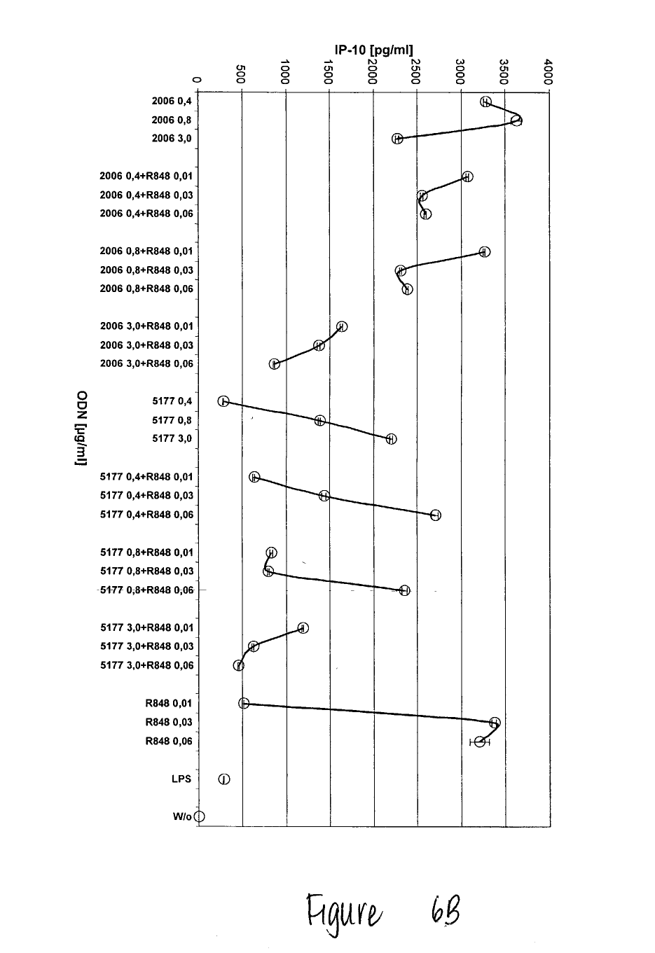

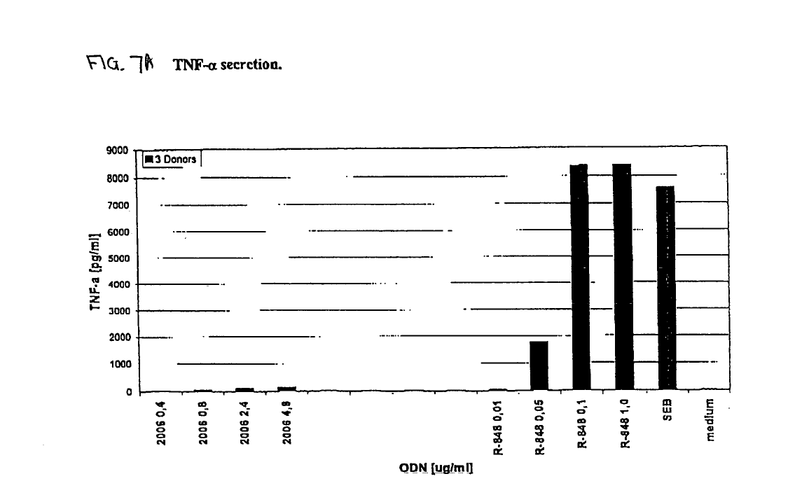

This experiment investigated the induction of the Th1 cytokine IFN-gamma as well as the Th1-related chemokine IP-10 (IFN-gamma inducible protein). Unfractionated human PBMC from three donors were incubated for 48 hours in the presence of varying concentrations of R-848 (0.01-1.0 μg/ml), varying concentrations of CpG ODN 2006 (0.2-3.0 μg/ml), varying concentrations of negative control ODN 5177 (0.2-3.0 μg/ml), SEB (50 ng/ml), or media alone, and then the concentrations of IP-10 and IFN-gamma in the supernatant were measured by ELISA. CpG ODN 2006 but not the negative control ODN 5177 induced similar amounts of IP-10 compared to R-848 (FIG. 6A). The same result was obtained for IFN-gamma (not shown).

In FIG. 6B, the combined effects of R-848 and CpG ODN (e.g., #2006) on IP-10 secretion are shown. Human PBMCs from three different donors were incubated for 48 hours with the indicated concentrations of ODNs and R-848, either individually or together. Supernatants were harvested and IP-10 was measured by ELISA. The data represent mean cytokine amounts. The data suggest that a dose-dependent negative effect of IP-10 secretion results from the use of certain CpG ODNs together with R-848.

### Example 6

R-848 is a More Potent Inducer of Pro-inflammatory Cytokines than CpG ODN

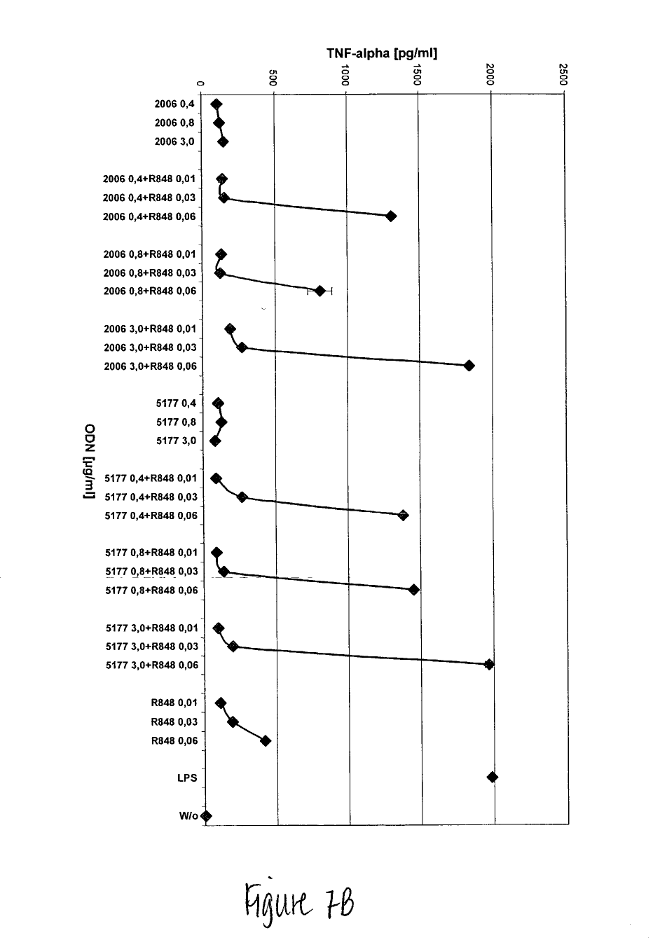

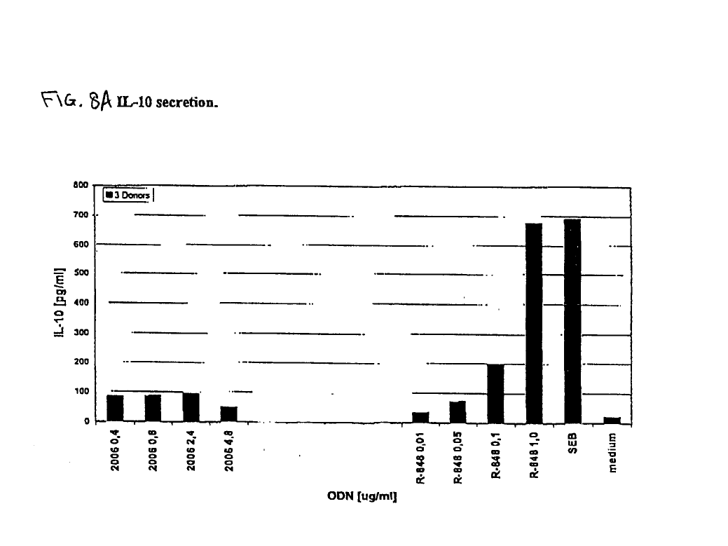

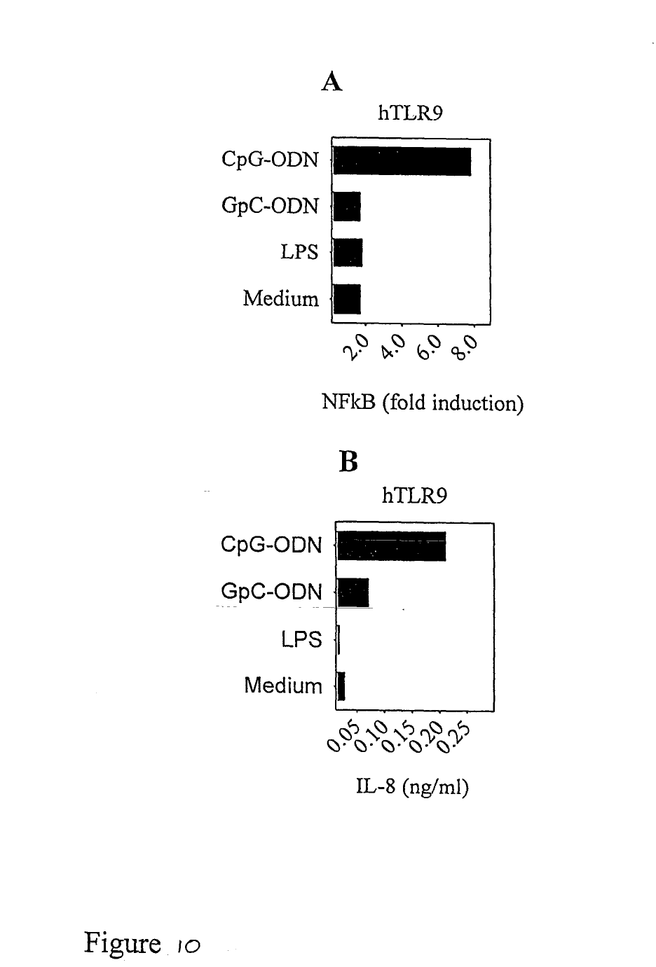

CpG ODNs are described to induce low but significant amounts of pro-inflammatory cytokines such as TNF-alpha and IL-6. Unfractionated human PBMC from three donors were incubated for 48 hours in the presence of varying concentrations of R-848 (0.01-1.0 μg/ml), varying concentrations of CpG ODN 2006 (0.4-4.8 μg/ml), SEB (50 ng/ml), or media alone, and then the concentrations of TNF-alpha and IL-6 in the supernatant were measured by ELISA. R-848 was much more potent than any CpG ODN in inducing very high amounts of TNF-alpha (FIG. 7A) and also high amounts of IL-6 (FIG. 9). This feature represents a significant difference in the activities of CpG ODNs and imidazoquinolines.

In FIG. 7B, the combined effects of R-848 and CpG ODN (e.g., #2006) on TNF-alpha secretion are shown. Human PBMCs from two different donors were incubated for 16 hours with the indicated concentrations of ODNs and R-848, either individually or together. Supernatants were harvested and TNF-alpha was measured by ELISA. The data represent mean cytokine amounts. The data suggest that a synergistic response, as the amounts of TNF-alpha secreted following incubation with both CpG ODNs and R-848 is greater than the additive amount secreted with either compound alone.

### Example 7

R-848 Induces IL-10

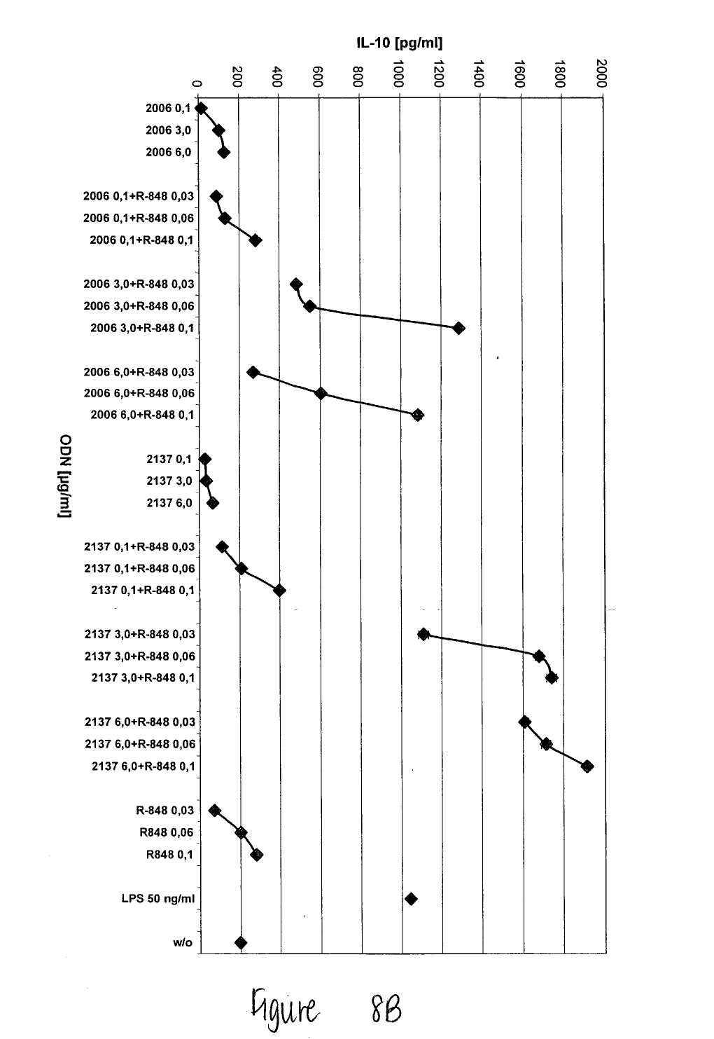

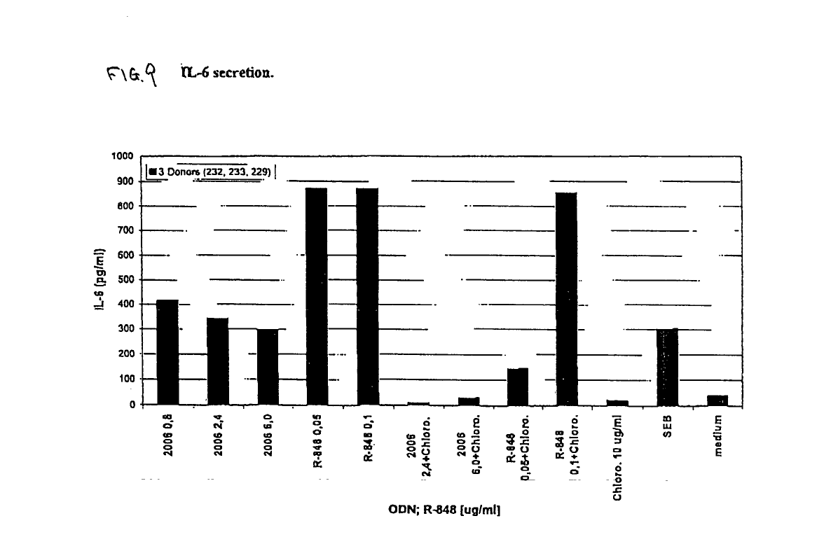

IL-10 represents a putative negative regulator of immunostimulation and is widely believed to antagonize the production of the Th1 cytokines IFN-gamma and IL-12. Unfractionated human PBMC from three donors were incubated for 48 hours in the presence of varying concentrations of R-848 (0.01-1.0 μg/ml), varying concentrations of CpG ODN 2006 (0.4-4.8 μg/ml), SEB (50 ng/ml), or media alone, and then the concentrations of IL-10 in the supernatant was measured by ELISA. R-848 induced higher amounts of IL-10 than CpG ODN 2006 (FIG. 8A).

In FIG. 8B, the combined effects of R-848 and CpG ODN (e.g., #2006) on IL-10 secretion are shown. Human PBMCs from two different donors were incubated for 48 hours with the indicated concentrations of ODNs and R-848, either individually or together. Supernatants were harvested and IL-10 was measured by ELISA. The data represent mean cytokine amounts. The data suggest that a synergistic response, as the amounts of IL-10 secreted following incubation with both CpG ODNs and R-848 is greater than the additive amount secreted with either compound alone.

### Example 8

Type B CpG ODN, But Not R-848, Can Be Fully Inhibited by Chloroquine

Vasilakos et al. reported that the activity of R-848 can not be inhibited by chloroquine, a compound blocking endosomal maturation. Vasilakos J P et al. (2000) Cell Immunol 204:64-74. Human PBMC (n=3) were cultured for 24 h with varying concentrations of R-848 (0.050.1 μg/ml), varying concentrations of CpG ODN 2006 (0.8-6.0 μg/ml), SEB (50 ng/ml), or media alone. In addition, PBMC were incubated with R-848 or ODN in the presence of 10 μg/ml chloroquine. IL-6 in the supernatants was measured by ELISA. Chloroquine blocked more than 90% of the activity of type B CpG ODNs, that interact with TLR9. TLR9 is believed to have intracellular expression only. These results, in contrast to the report of Vasilakos et al., demonstrate that the activity of R-848 can be strongly but not fully inhibited by chloroquine, dependent on the R-848 concentration (FIG. 9). A similar result was also obtained for B cell activation (not shown).

### Example 9

Reconstitution of TLR9 Signaling in 293 Fibroblasts

Methods for cloning murine and human TLR9 have been described in pending U.S. patent application Ser. No. 09/954,987, filed Sep. 17, 2001, and published PCT application PCT/US01/29229, the contents of which are incorporated by reference. Human TLR9 cDNA (SEQ ID NO:6, GenBank Accession No. AF245704) and murine TLR9 cDNA (SEQ ID NO:8, GenBank Accession No. AF348140) in pT-Adv vector (from Clontech) were individually cloned into the expression vector pcDNA3.1 (−) from Invitrogen using the EcoRI site. Utilizing a “gain of function” assay it was possible to reconstitute human TLR9 (hTLR9) and murine TLR9 (mTLR9) signaling in CpG-DNA non-responsive human 293 fibroblasts (ATCC, CRL-1573). The expression vectors mentioned above were transfected into 293 fibroblast cells using the calcium phosphate method. The amino acid sequence of human TLR9 is provided as SEQ ID NO:7 (GenBank Accession No. AAF78037). The amino acid sequence of murine TLR9 is provided as SEQ ID NO:9 (GenBank Accession No. AAK29625).

Since NF-kappa B activation is central to the IL-1/TLR signal transduction pathway (Medzhitov R et al. (1998) Mol Cell 2:253-258 (1998); Muzio M et al. (1998) J Exp Med 187:2097-101), cells were transfected with hTLR9 or co-transfected with hTLR9 and an NF-kappa B-driven luciferase reporter construct. Human fibroblast 293 cells were transiently transfected with (FIG. 10A) hTLR9 and a six-times NF-kappa B-luciferase reporter plasmid (NF-kappa B-luc, kindly provided by Patrick Baeuerle, Munich, Germany) or (FIG. 10B) with hTLR9 alone. After stimulus with CpG-ODN (2006, 2 μM, TCGTCGTTTTGTCGTTTTGTCGTT, SEQ ID NO:1), GpC-ODN (2006-GC, 2 μM, TGCTGCTTTTGTGCTTTTGTGCTT, SEQ ID NO:10), LPS (100 ng/ml) or media, NF-kappa B activation by luciferase readout (8 h, FIG. 10A) or IL-8 production by ELISA (48 h, FIG. 10B) were monitored. Results are representative of three independent experiments. FIG. 10 shows that cells expressing hTLR9 responded to CpG-DNA but not to LPS.

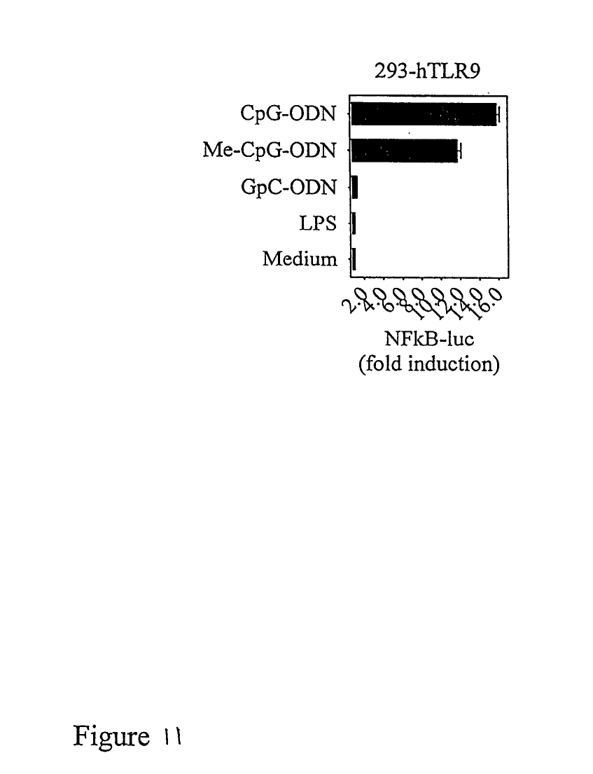

FIG. 11 demonstrates the same principle for the transfection of mTLR9. Human fibroblast 293 cells were transiently transfected with mTLR9 and the NF-kappa B-luc construct (FIG. 11). Similar data was obtained for IL-8 production (not shown). Thus expression of TLR9 (human or mouse) in 293 cells results in a gain of function for CpG-DNA stimulation similar to hTLR4 reconstitution of LPS responses.

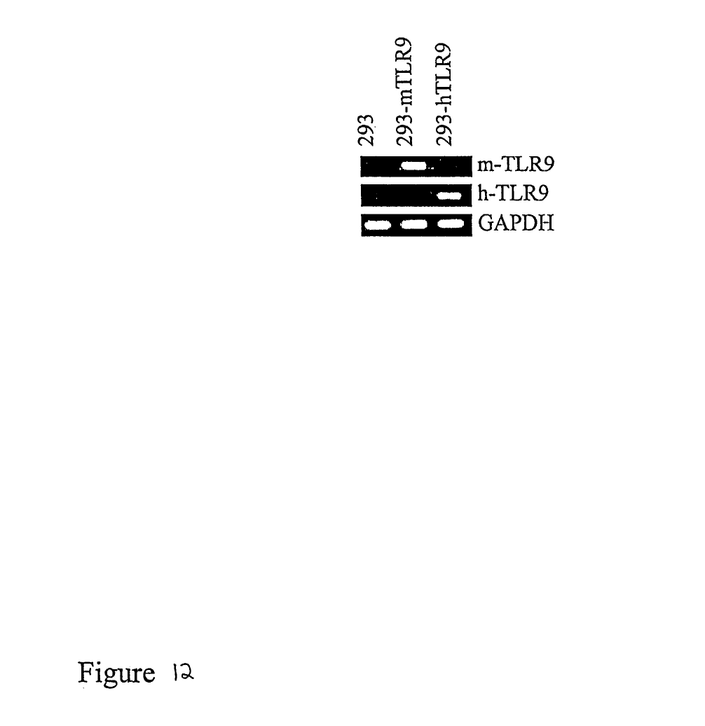

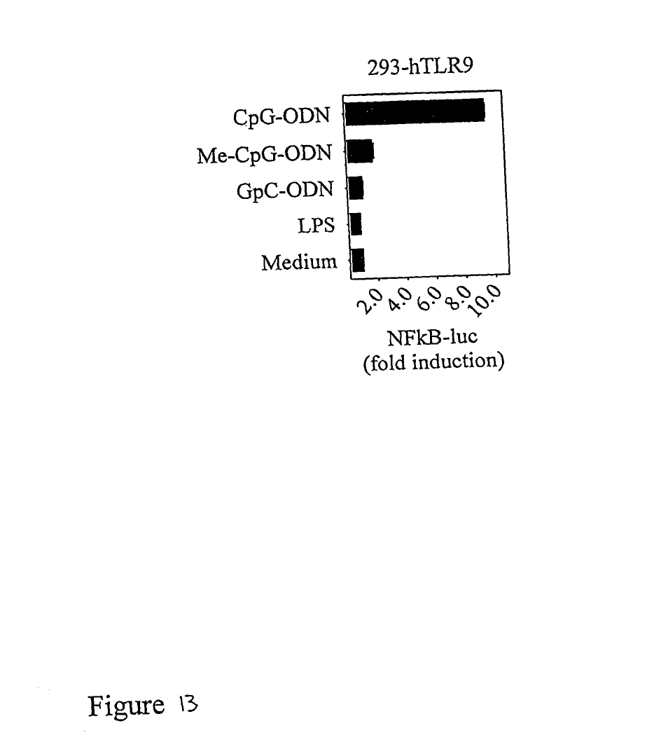

To generate stable clones expressing human TLR9, murine TLR9, or either TLR9 with the NF-kappa B-luc reporter plasmid, 293 cells were transfected in 10 cm plates (2×10⁶ cells/plate) with 16 μg of DNA and selected with 0.7 mg/ml G418 (PAA Laboratories GmbH, Cölbe, Germany). Clones were tested for TLR9 expression by RT-PCR, for example as shown in FIG. 12. The clones were also screened for IL-8 production or NF-kappa B-luciferase activity after stimulation with ODN. Four different types of clones were generated.

293-hTLR9-Luc: expressing human TLR9 and 6-fold NF-kappa B-luciferase reporter

293-mTLR9-Luc: expressing murine TLR9 and 6-fold NF-kappa B-luciferase reporter

293-hTLR9: expressing human TLR9

293-mTLR9: expressing murine TLR9

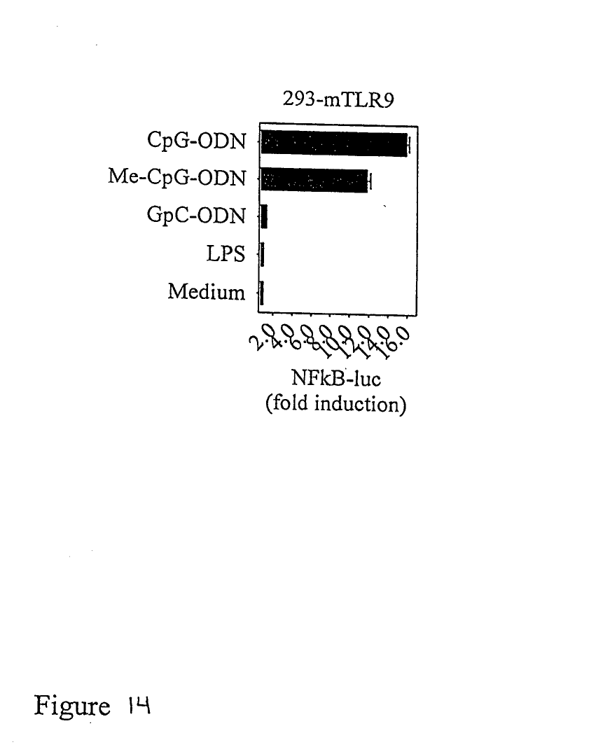

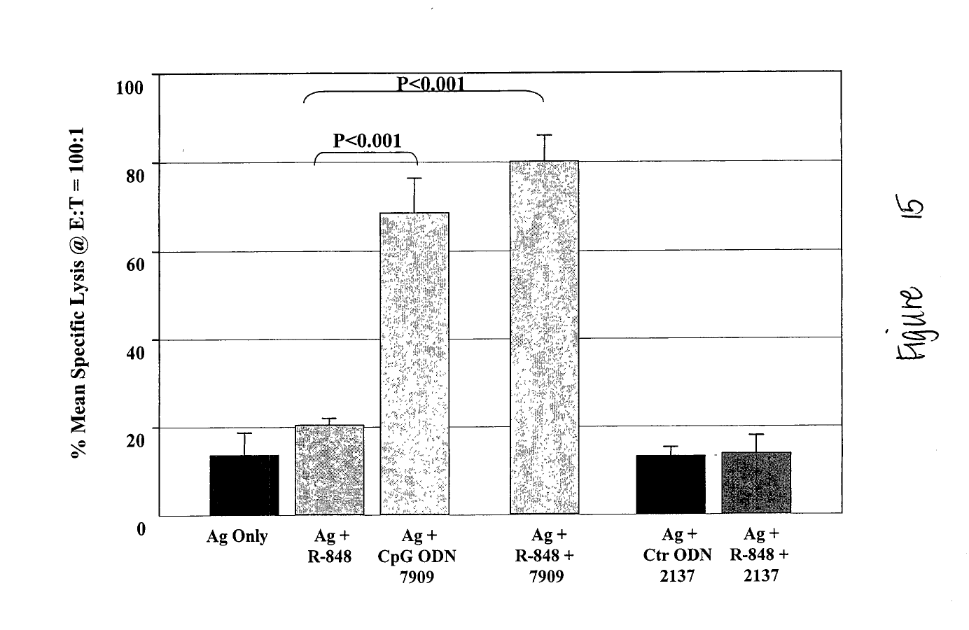

FIG. 13 demonstrates the responsiveness of a stable 293-hTLR9-Luc clone after stimulation with CpG-ODN (2006, 2 μM), GpC-ODN (2006-GC, 2 μM), Me-CpG-ODN (2006 methylated, 2 μM; TZGTZGTTTTGTZGTTTTGTZGTT, Z=5-methylcytidine, SEQ ID NO: 11), LPS (100 ng/ml) or media, as measured by monitoring NF-kappa B activation. Similar results were obtained utilizing IL-8 production with the stable clone 293-hTLR9. 293-mTLR9-Luc were also stimulated with CpG-ODN (1668, 2 μM; TCCATGACGTTCCTGATGCT, SEQ ID NO:12), GpC-ODN (1668-GC, 2 μM; TCCATGAGCTTCCTGATGCT, SEQ ID NO:13), Me-CpG-ODN (1668 methylated, 2 μM; TCCATGAZGTTCCTGATGCT, Z=5-methylcytidine, SEQ ID NO:14), LPS (100 ng/ml) or media, as measured by monitoring NF-kappa B activation (FIG. 14). Similar results were obtained utilizing IL-8 production with the stable clone 293-mTLR9. Results are representative of at least two independent experiments. These results demonstrate that CpG-DNA non-responsive cell lines can be stably genetically complemented with TLR9 to become responsive to CpG-DNA in a motif-specific manner. These cells can be used for screening of optimal ligands for innate immune responses driven by TLR9 in multiple species.

### Example 10

Method of Cloning Human TLR7

Two accession numbers in the GenBank database, AF245702 and AF240467, describe the DNA sequence for human TLR7. To create an expression vector for human TLR7, human TLR7 cDNA was amplified from a cDNA made from human peripheral mononuclear blood cells (PBMC) using the primers 5′-CACCTCTCATGCTCTGCTCTCTTC-3′ (SEQ ID NO:15) and 5′-GCTAGACCGTTTCCTTGAACACCTG-3′ (SEQ ID NO:16). The fragment was cloned into pGEM-T Easy vector (Promega), cut with the restriction enzyme NotI and ligated into a NotI-digested pCDNA3.1 expression vector (Invitrogen). The insert was fully sequenced and translated into protein. The cDNA sequence for hTLR7 is provided as SEQ ID NO:17. The open reading frame starts at base 124, ends at base 3273, and codes for a protein of 1049 amino acids (SEQ ID NO:18, Table 6).

The protein sequence of the cloned hTLR7 cDNA matches the sequence described under the GenBank accession number AF240467. The sequence deposited under GenBank accession number AF245702 contains two amino acid changes at position 725 (L to H) and 738 (L to P).

### Example 11

Method of Cloning Murine TLR7

Alignment of human TLR7 protein sequence with mouse EST database using tfasta yielded 4 hits with mouse EST sequences BB116163, AA266744, BB210780 and AA276879. Two primers were designed that bind to AA266744 sequence for use in a RACE-PCR to amplify 5′ and 3′ ends of the murine TLR7 cDNA. The library used for the RACE PCR was a mouse spleen marathon-ready cDNA commercially available from Clontech. A 5′ fragment with a length of 3000 bp obtained by this method was cloned into Promega pGEM-T Easy vector. After sequencing of the 5′ end, additional primers were designed for amplification of the complete murine TLR7 cDNA. The primer for the 5′ end was obtained from the sequence of the 5′ RACE product whereas the primer for the 3′ end was selected from the mouse EST sequence aa266744.

Three independent PCR reactions were set up using a murine macrophage RAW264.7 (ATCC TIB-71) cDNA as a template with the primers 5′-CTCCTCCACCAGACCTCTTGATTCC-3′ (SEQ ID NO:19) and 5′-CAAGGCATGTCCTAGGTGGTGACATTC-3′ (SEQ ID NO:20). The resulting amplification products were cloned into pGEM-T Easy vector and fully sequenced (SEQ ID NO:21). The open reading frame of mTLR7 starts at base 49, ends at base 3201 and codes for a protein of 1050 amino acids (SEQ ID NO:22). To create an expression vector for murine TLR7 cDNA, pGEM-T Easy vector plus mTLR7 insert was cut with NotI, the fragment isolated and ligated into a NotI digested pCDNA3.1 expression vector (Invitrogen).

### Example 12

Method of Cloning Human TLR8

Two accession numbers in the GenBank database, AF245703 and AF246971, describe the DNA sequence for human TLR8. To create an expression vector for human TLR8, human TLR8 cDNA was amplified from a cDNA made from human peripheral mononuclear blood cells (PBMC) using the primers 5′-CTGCGCTGCTGCAAGTTACGGAATG-3′ (SEQ ID NO:23) and 5′-GCGCGAAATCATGACTTAACGTCAG-3 (SEQ ID NO:24). The fragment was cloned into pGEM-T Easy vector (Promega), cut with the restriction enzyme NotI and ligated into a NotI-digested pCDNA3.1 expression vector (Invitrogen). The insert was fully sequenced and translated into protein. The cDNA sequence for hTLR8 is provided as SEQ ID NO:25. The open reading frame starts at base 83, ends at base 3208, and codes for a protein of 1041 amino acids (SEQ ID NO:26).

The protein sequence of the cloned hTLR8 cDNA matches the sequence described under the GenBank accession number AF245703. The sequence deposited under GenBank accession number AF246971 contains an insertion at the N-terminus of 15 amino acids (MKESSLQNSSCSLGKETKK; SEQ ID NO:27) and three single amino acid changes at positions 217 (P to S), 266 (L to P) and 867 (V to I).

### Example 13

Method of Cloning Murine TLR8

Alignment of human TLR8 protein sequence with mouse EST database using tfasta yielded 1 hit with mouse EST sequence BF135656. Two primers were designed that bind to BF135656 sequence for use in a RACE-PCR to amplify 5′ and 3′ ends of the murine TLR8 cDNA. The library used for the RACE PCR was a mouse spleen marathon-ready cDNA commercially available from Clontech. A 5′ fragment with a length of 2900 bp and a 3′ fragment with a length of 2900 bp obtained by this method were cloned into Promega pGEM-T Easy vector. After sequencing of the 5′ end and 3′ end of each fragment, partial sequences of mTLR8 were obtained and allowed the design of primers for amplification of the complete murine TLR8 cDNA.

Three independent PCR reactions were set up using a spleen murine cDNA from Clontech as a template with the primers 5′-GAGAGAAACAAACGTTTTACCTTC-3′ (SEQ ID NO:28) and 5′-GATGGCAGAGTCGTGACTTCCC-3′ (SEQ ID NO:29). The resulting amplification products were cloned into pGEM-T Easy vector, fully sequenced, translated into protein, and aligned to the human TLR8 protein sequence (GenBank accession number AF245703). The cDNA sequence for mTLR8 is provided as SEQ ID NO:30. The open reading frame of mTLR8 starts at base 59, ends at base 3157, and codes for a protein of 1032 amino acids (SEQ ID NO:31). To create an expression vector for murine TLR8, cDNA pGEM-T Easy vector with the mTLR8 insert was cut with NotI, the fragment isolated, and ligated into a NotI-digested pCDNA3.1 expression vector (Invitrogen).

### Example 14

Transient Transfectants Expressing TLR8 and TLR7

The cloned human TLR7 and human TLR8 cDNA were cloned into the expression vector pCDNA3.1(−) from Invitrogen using the NotI site. Utilizing a “gain of function” assay, hTLR7 and hTLR8 expression vectors were transiently expressed in human 293 fibroblasts (ATCC, CRL-1573) using the calcium phosphate method. Activation was monitored by IL-8 production after stimulus with CpG-ODN (2006 or 1668, 2 μM) or LPS (100 ng/ml). None of the stimuli used activated 293 cells transfected with either hTLR7 or hTLR8.

### Example 15

In Vivo Comparisons of CpG ODNs and R-848

CpG ODN (e.g., #7909) and imidazoquinoline compounds (e.g., R-848) were compared for their ability to augment antigen specific immune responses. Imidazoquinoline compounds Imiquimod (R-847) and Resiquimod (R-848) are shown to be topically active immune response modifiers and have been shown to induce production of IFN-α, IFN-γ, TNF-α and IL-12 in cultured human blood mononuclear cells. They have also been shown to possess both anti viral and anti tumor properties. A recent study by Vasilakos et al. (2000) has shown that R-848 is a strong Th1 biased adjuvant and, like CpG ODN, can re-direct Th2 biased immune responses established by alum. This study was aimed at comparing CpG ODN (7909) and R-848 for their potential use as vaccine adjuvants and to determine whether it is possible to obtain stronger immune responses by combining the 2 adjuvants. The study used HBsAg as a model antigen and evaluated the augmentation of both antigen specific humoral (i.e., antibody) and cell mediated (i.e., CTL, IFN-γ secretion) immune responses

Nucleic Acids and Imidazoquinoline Compounds: CpG ODN 7909 (GMP quality) and the non CpG control ODN 2137 were used. All ODN were re-suspended in sterile, endotoxin free TE at pH 8.0 (OmniPer®; EM Science, Gibbstown, N.J.) and stored and handled under aseptic conditions to prevent both microbial and endotoxin contamination. R-848 was manufactured by GL synthesis (Boston, Mass.) and was dissolved in TE buffer (pH 8.0) containing 10% DMSO. Dilution of ODNs and R-848 for assays was carried out in sterile, endotoxin free PBS a pH 7.2 (Sigma Chemical Company, St. Lois, Mo.).

Animals: Female BALB/c mice (6-8 weeks of age) were used for all experiments.

Animals were purchased from Charles River Canada (Quebec, Canada) and housed in micro-isolators at the animal care facility of the Ottawa Hospital Research Institute, Civic Site.

Immunization of mice: BALB/c mice (n=10/group) were immunized with 1 μg HBsAg sub type ad (International Enzymes, Calif.) alone, or in combination with CpG ODN 7909 (10 μg), control ODN 2137 (10 μg), R-848 (0.1, 1.0, 10 or 20 μg), or combinations of R-848 (20 μg)+ODN (10 μg). Animals were bled and boosted at 4 weeks post-primary immunization. At this time, 5 animals from each group were euthanized and spleens removed for CTL assays. Animals were also bled at 2 weeks post boost.

Determination of antibody responses: Antibodies (total IgG, IgG1 and IgG2a) specific to HBsAg (anti-HBs) were detected and quantified by endpoint dilution ELISA assay, which was performed in triplicate on samples from individual animals. Davis et al. J. Immunol 160: 870 (1998). End-point titers were defined as the highest plasma dilution that resulted in an absorbance value (OD 450) two times greater than that of non-immune plasma with a cut-off value of 0.05. These were reported as group mean titers±SEM.

Evaluation of CTL responses. CTL assays were conducted as previously described. McCluskie et al. J. Immunol 161:4463 (1998). Briefly, spleens were removed at 4 weeks post immunization and homogenized into single cell suspension in RPMI 1640 (Life Technologies, Grand Island, N.Y.) tissue culture medium supplemented with 10% fetal bovine serum (Life Technologies), penicillin-streptomycin solution (final concentration of 1000 U/ml and 1 mg/ml respectively; Sigma, Irvine, UK), and 5×10⁻⁵ M β-mercaptoethanol (Sigma) (Complete RPMI 1640). HBsAg specific lymphocytes in splenocyte suspensions (3×10⁶ cells/ml) were re-stimulated for 5 days by incubating with a murine cell line (p815-S) expressing HBsAg. Following re-stimulation, the potential of the lymphocytes to kill cells expressing HBsAg was determined by using ⁵¹Cr release assay. The results are presented as % specific lysis at different effector: target (E:T) ratios.

Cytokine secretion profile: Cytokine secretion profiles were measured following antigen re-stimulation of splenocytes from immunized animals. Spleen cell suspensions were prepared and adjusted to a final concentration of 5×10⁶ cells per ml in RPMI 1640 (Life Technologies, Grand Island, N.Y.) tissue culture medium supplemented with 2% normal mouse serum (Cedarlane Laboratories, Ontario, Canada), penicillin-streptomycin solution (final concentration of 1000 U/ml and 1 mg/ml respectively; Sigma, Irvine, UK), and 5×10⁻⁵ M β-mercaptoethanol (Sigma) (Complete RPMI 1640). Splenocyte suspension was plated onto 96-well U-bottom tissue culture plates (100 μl/well) along with 100 μl of each stimulant diluted to appropriate concentrations in Complete RPMI 1640. The stimulant used was HBsAg at 5 and 2.5 μg/ml. Concanavalin A (10 μg/ml, Sigma) was used as a positive control and cells cultured with media alone were used as negative controls. Each splenocyte sample was plated in triplicate and the cells were incubated in a humidified 5% CO₂ incubator at 37° C. for 48 and 72 hr. At the end of the incubation period, the 96-well plates were centrifuged for 5 min at 1200 rpm and culture supernatants harvested and stored at −80° C. until assayed. Commercially available assay kits (mouse IL-4 OptEIA, and mouse IFN-γ OptEIA; PharMingen, Mississauga, ON) were used according to manufacturer's instructions to assay cytokine levels in culture supernatants taken at 48 hr (IL-4) and 72 hr (IFN-γ).

Statistical analysis: Statistical analysis was performed using InStat program (Graph PAD Software, San Diego). The statistical difference between groups were determined by Student's t test (for two groups) or by 1-factor ANOVA followed by Tukey's test (for three or more groups) on raw data or transformed data (log10, for heteroscedastic populations).

Results:

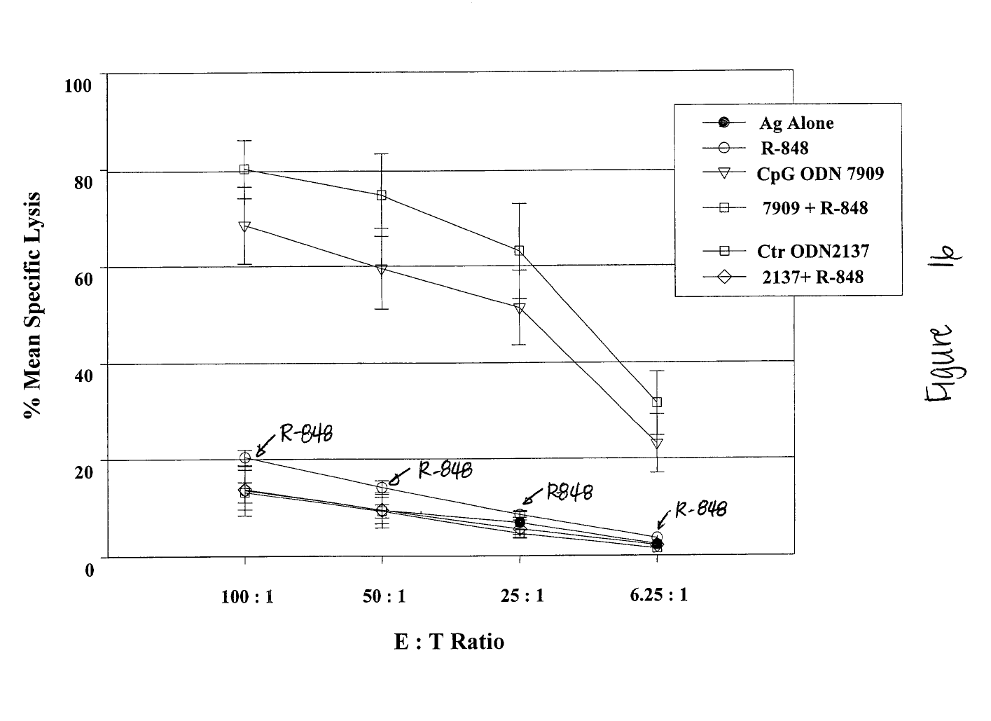

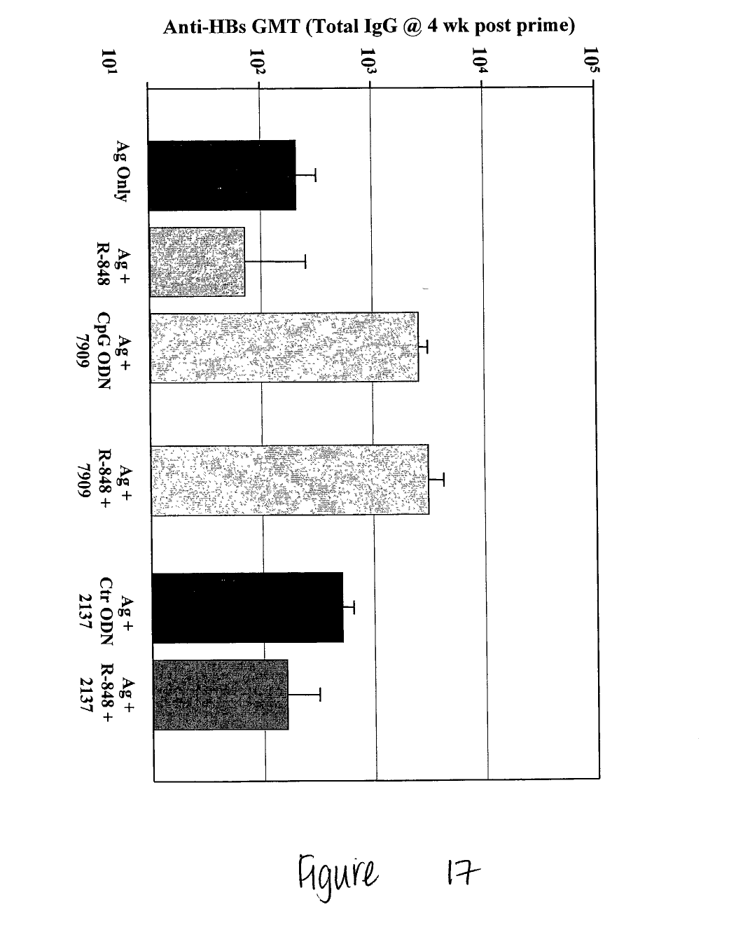

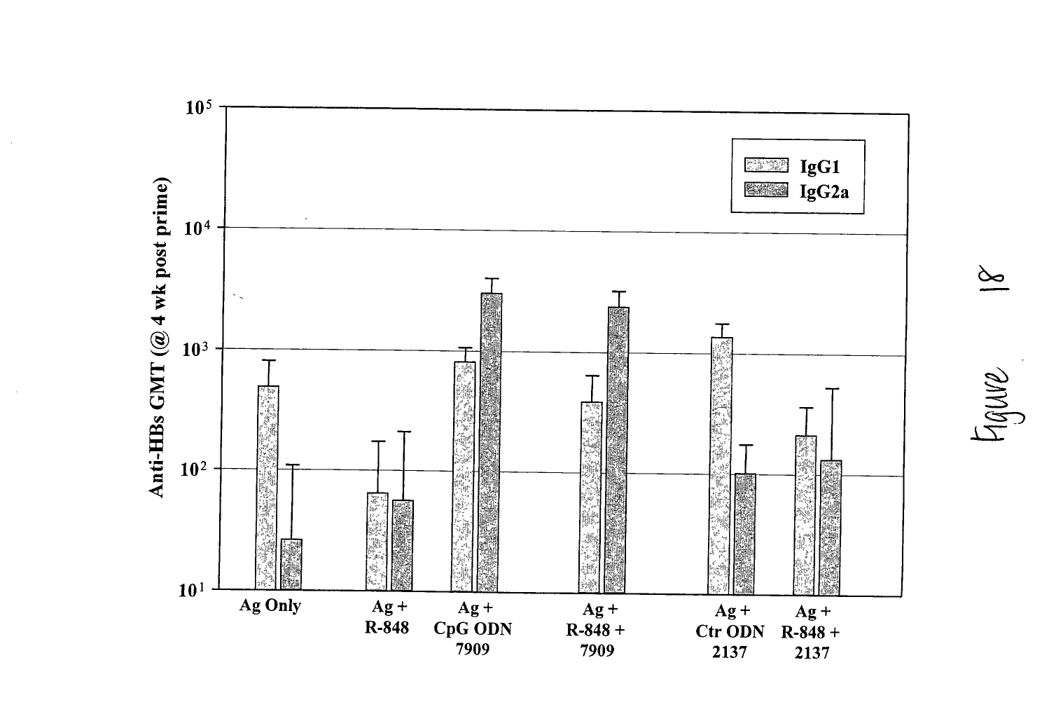

CpG ODNs and R-848 were tested either together or individually for their ability to augment a cytolytic T lymphocyte response against antigen (e.g., HbsAg) in vivo. CTL activity was measured at 4 weeks post prime. R-848 was able to augment the CTL response over antigen alone, however it was not as effective as CpG ODN (e.g., #7909). The combination of R-848 and CpG ODN together resulted in at least an additive effect. No augmentation of the CTL response over antigen alone was observed using control ODN either alone or with R-848. (See FIG. 15.) The data of FIG. 15 are plotted as a function of effector to target ratios in FIG. 16.

CpG ODNs and R-848 were tested either together or individually for their ability to augment an antibody response against antigen (e.g., HbsAg) in vivo. Anti-HbsAg antibody levels were measured at 4 weeks post prime. The antibody response in the presence of CpG ODN either with or without R-848 was similar.

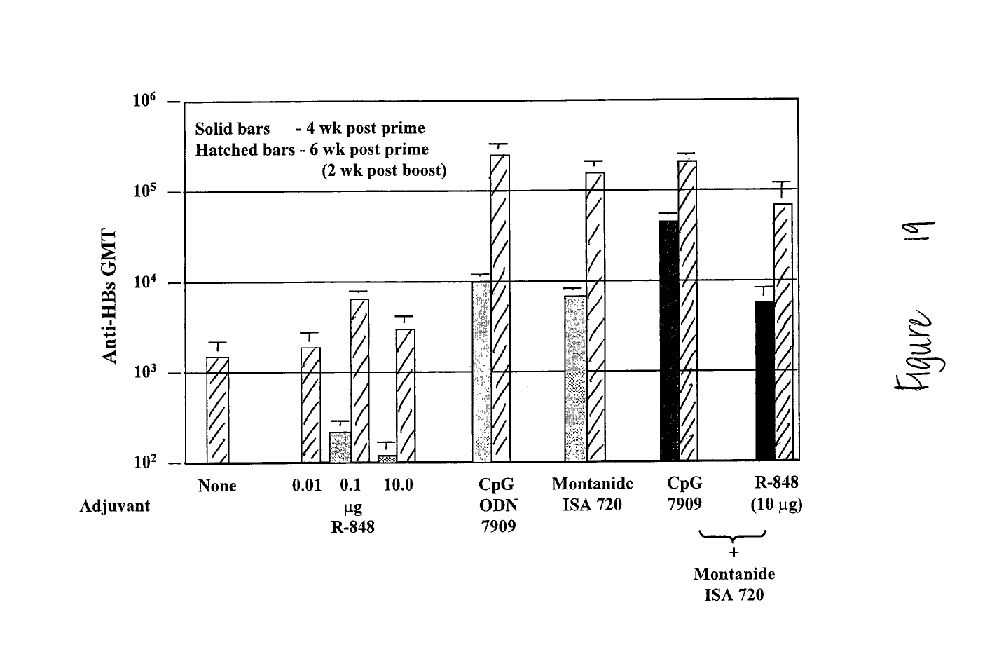

In FIG. 18, the distribution of antibody isotype is shown. While antigen alone produced higher levels of IgG1 antibody (as did control ODN with antigen), CpG ODN produced higher levels of IgG2a antibodies regardless of whether R-848 was present. R-848 appeared to increase the level of IgG2a and decrease the level of IgG1 as compared to the antigen alone response. A higher IgG2A/IgG1 ratio was observed at 6 weeks post prime using higher doses of R-848 (e.g., comparing 0.01 μg to 0.1 μg to 10.0 μg) (data not shown).

Splenocytes from immunized animals were assayed for antigen specific secretion of IFN-γ (Th1 like) and IL-4 (Th2 like) cytokines. No IL-4 was detected from any of the splenocyte cultures. However, splenocytes from animals immunized with HBsAg using CpG ODN 7909 as adjuvant induced high levels of IFN-γ secretion (data not shown).

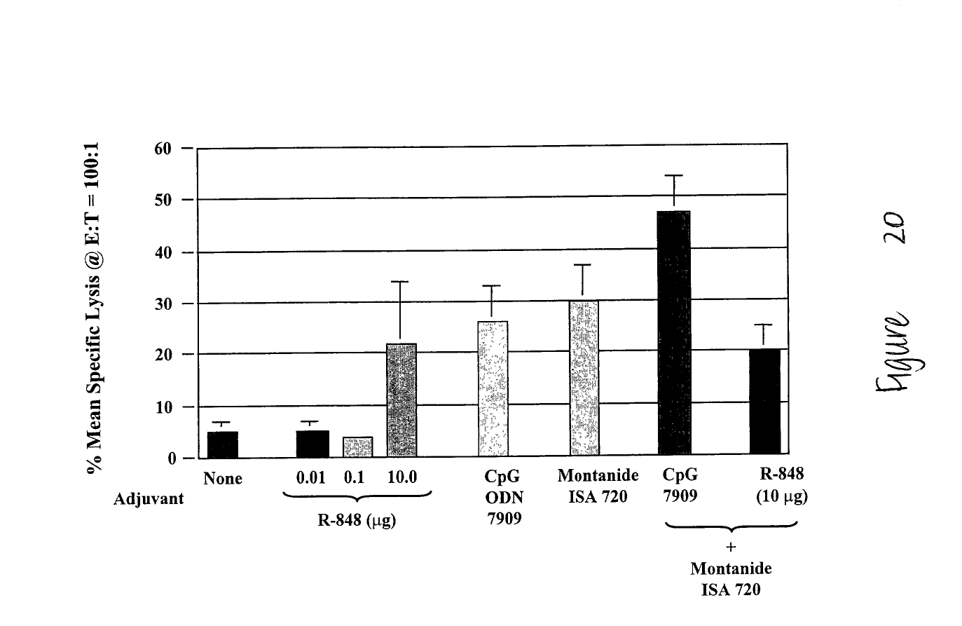

In FIG. 19, the effect of R-848, montanide ISA 720 and CpG ODN on augmentation of antibody responses against antigen (e.g., HbsAg) is compared. 6-8 week old BALB/c mice were immunized with 1 μg HbsAg alone or in combination with increasing doses of R-848, 10 μg CpG ODN, 70:30 (v/v) of antigen:montanide ISA 720, montanide and CpG ODN, or montanide and R-848. Anti-HbsAg levels were measured at 4 weeks post prime and at 2 weeks post boost (i.e., 6 weeks post prime). Montanide ISA 720 did not appear to augment the CpG ODN effect. The presence of R-848 did not appear to augment the montanide ISA 720 response.

In FIG. 20, the effect of R-848, montanide ISA 720 and CpG ODN on augmentation of CTL responses against antigen (e.g., HbsAg) is compared. 6-8 week old BALB/c mice were immunized with 1 μg HbsAg alone or in combination with increasing doses of R-848, 10 μg CpG ODN, 70:30 (v/v) of antigen:montanide ISA 720, montanide and CpG ODN, or montanide and R-848. CTL levels were measured at 4 weeks post prime. The montanide ISA 720 response was decreased in the presence of R-848. Montanide ISA 720 augmented the CpG ODN response slightly.

Recent studies have shown that imidazoquinoline compounds R-848 and R-847 activate cells of the immune system via the Toll-like receptors 7 and 8 (TLR7 and TLR8). Jurk et al. Nat. Immunol. 3:499 (2002). CpG ODN has been shown to act via TLR9. Takeshita et al. J. Immunol. 167:3555 (2001); Chuang et al. J. Leukoc biol 71:538 (2002). In humans, TLR 7 and 9 are localized on plasmacytoid dendritic cells (PDC) whereas TLR 8 is localized on monocyte derived dendritic cells (MDC).

In mice reported to be deficient in TLR8, both TLR7 and 9 co-localize on the same cell types. This may explain the additive effects observed when R-848 and CpG ODN are used as combination adjuvants in a murine system. Synergistic activity is expected in humans when R-848 and CpG ODN are used as combination adjuvants, because of the functionality of TLR7, TLR8 and TLR9. Furthermore, R-848 may be a more potent adjuvant in humans since both TLR 7 and 8 are fully functional in human cells.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A method of stimulating antibody dependent cellular cytotoxicity in a subject, comprising
administering an antibody and an agent selected from the group consisting of an imidazoquinoline agent and an C8-substituted guanosine to a subject in need of such treatment in an amount effective to stimulate antibody dependent cellular cytotoxicity in the subject.

2. The method of claim 1, wherein the agent is imidazoquinoline agent.

3. The method of claim 2, further comprising administering an C8-substituted guanosine to the subject.

4. The method of claim 2, further comprising administering a poly-arginine to the subject.

5. The method of claim 1, further comprising administering an immunostimulatory nucleic acid to the subject.

6. The method of claim 5, wherein the immunostimulatory nucleic acid is selected from the group consisting of a CpG nucleic acid and a poly-G nucleic acid.

7. The method of claim 5, wherein the immunostimulatory nucleic acid is selected from the group consisting of a poly-T nucleic acid, a T-rich nucleic acid, a TG nucleic acid, a CpI nucleic acid and a methylated CpG nucleic acid.

8. The method of claim 5, wherein the immunostimulatory nucleic acid has a backbone modification that is selected from the group consisting of a phosphorothioate modification and a peptide modification.

9. The method of claim 5, wherein the immunostimulatory nucleic acid has a backbone that is chimeric.

10. The method of claim 1, wherein the antibody is selected from the group consisting of an anti-cancer antibody, an anti-viral antibody, an anti-bacterial antibody, an anti-fungal antibody, an anti-allergen antibody, and an anti-self antigen antibody.

11. The method of claim 1, wherein the subject has or is at risk of having a disorder selected from the group consisting of asthma/allergy, infectious disease, cancer and warts.

12. The method of claim 1, wherein the imidazoquinoline agent is administered prior to the antibody.

13. The method of claim 1, wherein the imidazoquinoline agent is an imidazoquinoline amine.

14. The method of claim 1, wherein the imidazoquinoline agent is selected from the group consisting of imiquimod/R-837 and S-28463/R-848.

15. The method of claim 1, wherein the amount effective to stimulate antibody dependent cellular cytotoxicity is a synergistic amount.

16. A method for modulating an immune response in a subject, comprising
administering to a subject in need of such treatment an immunostimulatory nucleic acid and an agent selected from the group consisting of an imidazoquinoline agent and an C8-substituted guanosine in an amount effective to modulate the immune response.

17. The method of claim 16, wherein the agent is an imidazoquinoline agent.

18. The method of claim 17, further comprising administering an C8-substituted guanosine to the subject.

19. The method of claim 16, wherein the immune response is a Th1 immune response.

20. The method of claim 16, wherein the immune response is antibody dependent cellular cytotoxicity.

21. The method of claim 16, wherein the immune response is an innate immune response.

22. The method of claim 16, wherein the immunostimulatory nucleic acid is selected from the group consisting of a CpG nucleic acid and a poly-G nucleic acid.

23. The method of claim 16, wherein the immunostimulatory nucleic acid is selected from the group consisting of a poly-T nucleic acid, a T-rich nucleic acid, a TG nucleic acid, a CpI nucleic acid and a methylated CpG nucleic acid.

24. The method of claim 16, wherein the immunostimulatory nucleic acid has a backbone modification that is selected from the group consisting of a phosphorothioate modification and a peptide modification.

25. The method of claim 16, wherein the immunostimulatory nucleic acid has a chimeric backbone.

26. The method of claim 16, wherein the imidazoquinoline agent is an imidazoquinoline amine.

27. The method of claim 16, wherein the imidazoquinoline agent is selected from the group consisting of imiquimod/R-837 and S-28463/R-848.

28. The method of claim 16, wherein the immune response is a local immune response.

29. The method of claim 16, wherein the immune response is a mucosal immune response.

30. The method of claim 16, wherein the immune response is a systemic immune response.

31. The method of claim 16, wherein the agent is administered prior to the immunostimulatory nucleic acid.

32. The method of claim 16, wherein the amount effective to modulate the immune response is a synergistic amount.

33. The method of claim 16, further comprising administering poly-arginine to the subject.

34. The method of claim 16, further comprising administering an C8-substituted guanosine to the subject.

35. The method of claim 16, further comprising administering a disorder-specific medicament to the subject.

36. The method of claim 35, wherein the disorder-order specific medicament is selected from the group consisting of a cancer medicament, an asthma/allergy a medicament, an infectious disease medicament, and a wart medicament.

37. The method of claim 36, wherein the cancer medicament is selected from the group consisting of a chemotherapeutic agent, an immunotherapeutic agent and a cancer vaccine.

38. The method of claim 36, wherein the asthma/allergy medicament is selected from the group consisting of steroids, immunomodulators, anti-inflammatory agents, bronchodilators, leukotriene modifiers, β2 agonists, and anti-cholinergics.

39. The method of claim 36, wherein the anti-microbial medicament is selected from the group consisting of an anti-bacterial agent, an anti-viral agent, an anti-fungal agent, and an anti-parasitic agent.

40. The method of claim 16, further comprising exposing the subject to an antigen and wherein the immune response is an antigen-specific immune response.

41. The method of claim 40, wherein the antigen is selected from the group consisting of a tumor antigen, a viral antigen, a bacterial antigen, a parasitic antigen, and a fungal antigen.

42. The method of claim 16, wherein the subject has or is at risk of developing an infectious disease.

43. The method of claim 16, wherein the subject has or is at risk of developing a cancer.

44. The method of claim 16, wherein the subject has or is at risk of developing asthma/allergy.

45. The method of claim 16, wherein the subject is an immunocompromised subject.

46. The method of claim 16, wherein the subject is elderly or an infant.

47. A composition, comprising
an imidazoquinoline agent, and
an immunostimulatory nucleic acid.

48. The composition of claim 47, further comprising poly-arginine.

49. The composition of claim 47, further comprising an antigen.

50. The composition of claim 47, further comprising an C8-substituted guanosine.

51. The composition of claim 47, wherein the immunostimulatory nucleic acid is a CpG nucleic acid.

52. The composition of claim 51, wherein the immunostimulatory nucleic acid is a poly-G nucleic acid.

53. The composition of claim 47, wherein the immunostimulatory nucleic acid is a T-rich nucleic acid.

54. A composition comprising
an imidazoquinoline agent and an antibody.

55. The composition of claim 54, further comprising poly-arginine.

56. The composition of claim 55, further comprising an immunostimulatory nucleic acid.

57. The composition of claim 54, further comprising an C8-substituted guanosine.

58. A composition, comprising
an imidazoquinoline agent and a disorder-specific medicament.

59. The composition of claim 58, wherein the disorder-specific medicament is selected from the group consisting of an asthma/allergy medicament, a cancer medicament, and an anti-microbial medicament.

60. The composition of claim 58, further comprising poly-arginine.

61. The composition of claim 58, further comprising an immunostimulatory nucleic acid.

62. The composition of claim 58, further comprising an C8-substituted guanosine.

63. The composition of claim 59, wherein the asthma/allergy medicament is selected from the group consisting of steroids, immunomodulators, anti-inflammatory agents, bronchodilators, leukotriene modifiers, β2 agonists, and anti-cholinergics.

64. The composition of claim 59, wherein the cancer medicament is selected from the group consisting of a chemotherapeutic agent, an immunotherapeutic agent and a cancer vaccine.

65. The composition of claim 59, wherein the anti-microbial medicament is selected from the group consisting of an anti-bacterial agent, an anti-viral agent, an anti-fungal agent, and an anti-parasitic agent.

66. A method for inducing an antigen-specific immune response in a subject comprising
administering to a subject an antigen, an imidazoquinoline agent, and an immunostimulatory nucleic acid in an effective amount to induce an antigen specific immune response.

67. The method of claim 66, wherein the antigen is selected from the group consisting of selected from the group consisting of a tumor antigen, a viral antigen, a bacterial antigen, a parasitic antigen, and a fungal antigen.

68. A screening method for comparing Toll-like receptor (TLR) signaling activity of a test compound with TLR signaling activity of an imidazoquinoline, comprising:
contacting a functional TLR selected from the group consisting of Toll-like receptor 7 (TLR7) and Toll-like receptor 8 (TLR8) with a reference imidazoquinoline and detecting a reference response mediated by a TLR signal transduction pathway;
contacting a functional TLR selected from the group consisting of TLR7 and TLR8 with a test compound and detecting a test response mediated by a TLR signal transduction pathway; and
comparing the test response with the reference response to compare the TLR signaling activity of the test compound with the imidazoquinoline.

69. The method of claim 68, wherein the functional TLR is TLR8.

70. The method of claim 68, wherein the functional TLR is TLR7.

71. The method of claim 68, wherein the functional TLR is contacted with the reference imidazoquinoline and the test compound independently.

72. The method of claim 71, wherein the screening method is a method for identifying an imidazoquinoline mimic, and wherein when the test response is similar to the reference response the test compound is an imidazoquinoline mimic.

73. The method of claim 68, wherein the functional TLR is contacted with the reference imidazoquinoline and the test compound concurrently to produce a test-reference response mediated by a TLR signal transduction pathway and wherein the test-reference response may be compared to the reference response.

74. The method of claim 73, wherein the screening method is a method for identifying an imidazoquinoline agonist, and wherein when the test-reference response is greater than the reference response the test compound is an imidazoquinoline agonist.

75. The method of claim 73, wherein the screening method is a method for identifying an imidazoquinoline antagonist, and wherein when the test-reference response is less than the reference response the test compound is an imidazoquinoline antagonist.

76. The method of claim 68, wherein the functional TLR is expressed in a cell.

77. The method of claim 76, wherein the cell is an isolated mammalian cell that naturally expresses functional TLR8.

78. The method of claim 77, wherein the cell comprises an expression vector comprising an isolated nucleic acid which encodes a reporter construct selected from the group consisting of interleukin 8 (IL-8), p40 subunit of interleukin 12 (IL-12 p40), nuclear factor kappa B-luciferase (NF-kappaB-luc), p40 subunit of interleukin 12-luciferase (IL-12 p40-luc), and tumor necrosis factor-luciferase (TNF-luc).

79. The method of claim 68, wherein the functional TLR is part of a cell-free system.

80. The method of claim 68, wherein the functional TLR is part of a complex with another TLR.

81. The method of claim 68, wherein the functional TLR is part of a complex with a non-TLR protein selected from the group consisting of myeloid differentiation factor 88 (MyD88), IL-1 receptor-associated kinase (IRAK), tumor necrosis factor receptor-associated factor 6 (TRAF6), IkappaB, NF-kappaB, and functional homologs and derivatives thereof.

82. The method of claim 68, wherein the reference imidazoquinoline is R-848 (Resiquimod).

83. The method of claim 68, wherein the reference imidazoquinoline is R-847 (Imiquimod).

84. The method of claim 68, wherein the test compound is not a nucleic acid molecule.

85. The method of claim 68, wherein the test compound is a polypeptide.

86. The method of claim 68, wherein the test compound is an imidazoquinoline other than R-848 or R-847.

87. The method of claim 68, wherein the test compound is a part of a combinatorial library of compounds.


SEQUENCE LISTING (31 sequences)

SEQ ID NO: 1 - DNA, 24 bp, Artificial Sequence
  Synthetic Oligonucleotide
tcgtcgttttgtcgttttgtcgtt

SEQ ID NO: 2 - DNA, 24 bp, Artificial Sequence
  Synthetic Oligonucleotide
tttttttttttttttttttttttt

SEQ ID NO: 3 - DNA, 20 bp, Artificial Sequence
  Synthetic Oligonucleotide
tccaggacttctctcaggtt

SEQ ID NO: 4 - DNA, 21 bp, Artificial Sequence
  Synthetic Oligonucleotide
ggggacgacgtcgtggggggg

SEQ ID NO: 5 - DNA, 19 bp, Artificial Sequence
  Synthetic Oligonucleotide
tccgcctgtgacatgcatt

SEQ ID NO: 6 - DNA, 3352 bp, Homo sapiens
aggctggtataaaaatcttacttcctctattctctgagccgctgctgcccctgtgggaagggacctcgagtgtgaagcatccttccctgtagctgctgtccagtctgcccgccagaccctctggagaagcccctgccccccagcatgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccgcaataagctggacctctaccacgagcactcattcacggagctaccgcgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccggctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatagccgtgagccggaatcctgcacggtgccacctccacactcacctcacctctgcctgcctggtctgaccctcccctgctcgcctccctcaccccacacctgacacagagca

SEQ ID NO: 7 - protein, 1032 aa, Homo sapiens [sequence omitted]

SEQ ID NO: 8 - DNA, 3200 bp, Mus musculus
tgtcagagggagcctcgggagaatcctccatctcccaacatggttctccgtcgaaggactctgcaccccttgtccctcctggtacaggctgcagtgctggctgagactctggccctgggtaccctgcctgccttcctaccctgtgagctgaagcctcatggcctggtggactgcaattggctgttcctgaagtctgtaccccgtttctctgcggcagcatcctgctccaacatcacccgcctctccttgatctccaaccgtatccaccacctgcacaactccgacttcgtccacctgtccaacctgcggcagctgaacctcaagtggaactgtccacccactggccttagccccctgcacttctcttgccacatgaccattgagcccagaaccttcctggctatgcgtacactggaggagctgaacctgagctataatggtatcaccactgtgccccgactgcccagctccctggtgaatctgagcctgagccacaccaacatcctggttctagatgctaacagcctcgccggcctatacagcctgcgcgttctcttcatggacgggaactgctactacaagaacccctgcacaggagcggtgaaggtgaccccaggcgccctcctgggcctgagcaatctcacccatctgtctctgaagtataacaacctcacaaaggtgccccgccaactgccccccagcctggagtacctcctggtgtcctataacctcattgtcaagctggggcctgaagacctggccaatctgacctcccttcgagtacttgatgtgggtgggaattgccgtcgctgcgaccatgcccccaatccctgtatagaatgtggccaaaagtccctccacctgcaccctgagaccttccatcacctgagccatctggaaggcctggtgctgaaggacagctctctccatacactgaactcttcctggttccaaggtctggtcaacctctcggtgctggacctaagcgagaactttctctatgaaagcatcaaccacaccaatgcctttcagaacctaacccgcctgcgcaagctcaacctgtccttcaattaccgcaagaaggtatcctttgcccgcctccacctggcaagttccttcaagaacctggtgtcactgcaggagctgaacatgaacggcatcttcttccgctcgctcaacaagtacacgctcagatggctggccgatctgcccaaactccacactctgcatcttcaaatgaacttcatcaaccaggcacagctcagcatctttggtaccttccgagcccttcgctttgtggacttgtcagacaatcgcatcagtgggccttcaacgctgtcagaagccacccctgaagaggcagatgatgcagagcaggaggagctgttgtctgcggatcctcacccagctccactgagcacccctgcttctaagaacttcatggacaggtgtaagaacttcaagttcaccatggacctgtctcggaacaacctggtgactatcaagccagagatgtttgtcaatctctcacgcctccagtgtcttagcctgagccacaactccattgcacaggctgtcaatggctctcagttcctgccgctgactaatctgcaggtgctggacctgtcccataacaaactggacttgtaccactggaaatcgttcagtgagctaccacagttgcaggccctggacctgagctacaacagccagccctttagcatgaagggtataggccacaatttcagttttgtggcccatctgtccatgctacacagccttagcctggcacacaatgacattcatacccgtgtgtcctcacatctcaacagcaactcagtgaggtttcttgacttcagcggcaacggtatgggccgcatgtgggatgaggggggcctttatctccatttcttccaaggcctgagtggcctgctgaagctggacctgtctcaaaataacctgcatatcctccggccccagaaccttgacaacctccccaagagcctgaagctgctgagcctccgagacaactacctatctttctttaactggaccagtctgtccttcctgcccaacctggaagtcctagacctggcaggcaaccagctaaaggccctgaccaatggcaccctgcctaatggcaccctcctccagaaactggatgtcagcagcaacagtatcgtctctgtggtcccagccttcttcgctctggcggtcgagctgaaagaggtcaacctcagccacaacattctcaagacggtggatcgctcctggtttgggcccattgtgatgaacctgacagttctagacgtgagaagcaaccctctgcactgtgcctgtggggcagccttcgtagacttactgttggaggtgcagaccaaggtgcctggcctggctaatggtgtgaagtgtggcagccccggccagctgcagggccgtagcatcttcgcacaggacctgcggctgtgcctggatgaggtcctctcttgggactgctttggcctttcactcttggctgtggccgtgggcatggtggtgcctatactgcaccatctctgcggctgggacgtctggtactgttttcatctgtgcctggcatggctacctttgctggcccgcagccgacgcagcgcccaagctctcccctatgatgccttcgtggtgttcgataaggcacagagcgcagttgcggactgggtgtataacgagctgcgggtgcggctggaggagcggcgcggtcgccgagccctacgcttgtgtctggaggaccgagattggctgcctggccagacgctcttcgagaacctctgggcttccatctatgggagccgcaagactctatttgtgctggcccacacggaccgcgtcagtggcctcctgcgcaccagcttcctgctggctcagcagcgcctgttggaagaccgcaaggacgtggtggtgttggtgatcctgcgtccggatgcccaccgctcccgctatgtgcgactgcgccagcgtctctgccgccagagtgtgctcttctggccccagcagcccaacgggcaggggggcttctgggcccagctgagtacagccctgactagggacaaccgccacttctataaccagaacttctgccggggacctacagcagaatagctcagagcaacagctggaaacagctgcatcttcatgcctggttcccgagttgctctgcctgc

SEQ ID NO: 9 - protein, 1032 aa, Mus musculus
MVLRRRTLHPLSLLVQAAVLAETLALGTLPAFLPCELKPHGLVDCNWLFLKSVPRFSAAASCSNITRLSLISNRIHHLHNSDFVHLSNLRQLNLKWNCPPTGLSPLHFSCHMTIEPRTFLAMRTLEELNLSYNGITTVPRLPSSLVNLSLSHTNILVLDANSLAGLYSLRVLFMDGNCYYKNPCTGAVKVTPGALLGLSNLTHLSLKYNNLTKVPRQLPPSLEYLLVSYNLIVKLGPEDLANLTSLRVLDVGGNCRRCDHAPNPCIECGQKSLHLHPETFHHLSHLEGLVLKDSSLHTLNSSWFQGLVNLSVLDLSENFLYESINHTNAFQNLTRLRKLNLSFNYRKKVSFARLHLASSFKNLVSLQELNMNGIFFRSLNKYTLRWLADLPKLHTLHLQMNFINQAQLSIFGTFRALRFVDLSDNRISGPSTLSEATPEEADDAEQEELLSADPHPAPLSTPASKNFMDRCKNFKFTMDLSRNNLVTIKPEMFVNLSRLQCLSLSHNSIAQAVNGSQFLPLTNLQVLDLSHNKLDLYHWKSFSELPQLQALDLSYNSQPFSMKGIGHNFSFVAHLSMLHSLSLAHNDIHTRVSSHLNSNSVRFLDFSGNGMGRMWDEGGLYLHFFQGLSGLLKLDLSQNNLHILRPQNLDNLPKSLKLLSLRDNYLSFFNWTSLSFLPNLEVLDLAGNQLKALTNGTLPNGTLLQKLDVSSNSIVSVVPAFFALAVELKEVNLSHNILKTVDRSWFGPIVMNLTVLDVRSNPLHCACGAAFVDLLLEVQTKVPGLANGVKCGSPGQLQGRSIFAQDLRLCLDEVLSWDCFGLSLLAVAVGMVVPILHHLCGWDVWYCFHLCLAWLPLLARSRRSAQALPYDAFVVFDKAQSAVADWVYNELRVRLEERRGRRALRLCLEDRDWLPGQTLFENLWASIYGSRKTLFVLAHTDRVSGLLRTSFLLAQQRLLEDRKDVVVLVILRPDAHRSRYVRLRQRLCRQSVLFWPQQPNGQGGFWAQLSTALTRDNRHFYNQNFCRGPTAE

SEQ ID NO: 10 - DNA, 24 bp, Artificial Sequence
  Synthetic Oligonucleotide
tgctgcttttgtgcttttgtgctt

SEQ ID NO: 11 - DNA, 24 bp, Artificial Sequence
  Synthetic Oligonucleotide
tngtngttttgtngttttgtngtt

SEQ ID NO: 12 - DNA, 20 bp, Artificial Sequence
  Synthetic Oligonucleotide
tccatgacgttcctgatgct

SEQ ID NO: 13 - DNA, 20 bp, Artificial Sequence
  Synthetic Oligonucleotide
tccatgagcttcctgatgct

SEQ ID NO: 14 - DNA, 20 bp, Artificial Sequence
  Synthetic Oligonucleotide
tccatgangttcctgatgct

SEQ ID NO: 15 - DNA, 24 bp, Homo sapiens
cacctctcatgctctgctctcttc

SEQ ID NO: 16 - DNA, 25 bp, Homo sapiens
gctagaccgtttccttgaacacctg

SEQ ID NO: 17 - DNA, 3373 bp, Homo sapiens
  CDS (124)..(3273)
agctggctagcgtttaaacgggccctctagactcgagcggccgcgaattcactagtgattcacctctcatgctctgctctcttcaaccagacctctacattccattttggaagaagactaaaaatggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccttttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctagaatcgaattcccgcggccgccactgtgctggatatctgcagaattccaccacactggactagtggatccgagctcggtaccaagcttaagtttaaaccgc

SEQ ID NO: 18 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 19 - DNA, 25 bp, Mus musculus
ctcctccaccagacctcttgattcc

SEQ ID NO: 20 - DNA, 27 bp, Mus musculus
caaggcatgtcctaggtggtgacattc

SEQ ID NO: 21 - DNA, 3243 bp, Mus musculus
  CDS (49)..(3201)
attctcctccaccagacctcttgattccattttgaaagaaaactgaaaatggtgttttcgatgtggacacggaagagacaaattttgatctttttaaatatgctcttagtttctagagtctttgggtttcgatggtttcctaaaactctaccttgtgaagttaaagtaaatatcccagaggcccatgtgatcgtggactgcacagacaagcatttgacagaaatccctgagggcattcccactaacaccaccaatcttacccttaccatcaaccacataccaagcatctctccagattccttccgtaggctgaaccatctggaagaaatcgatttaagatgcaattgtgtacctgttctactggggtccaaagccaatgtgtgtaccaagaggctgcagattagacctggaagctttagtggactctctgacttaaaagccctttacctggatggaaaccaacttctggagataccacaggatctgccatccagcttacatcttctgagccttgaggctaacaacatcttctccatcacgaaggagaatctaacagaactggtcaacattgaaacactctacctgggtcaaaactgttattatcgaaatccttgcaatgtttcctattctattgaaaaagatgctttcctagttatgagaaatttgaaggttctctcactaaaagataacaatgtcacagctgtccccaccactttgccacctaatttactagagctctatctttataacaatatcattaagaaaatccaagaaaatgattttaataacctcaatgagttgcaagttcttgacctaagtggaaattgccctcgatgttataatgtcccatatccgtgtacaccgtgtgaaaataattcccccttacagatccatgacaatgctttcaattcattgacagaattaaaagttttacgtttacacagtaattctcttcagcatgtgcccccaacatggtttaaaaacatgagaaacctccaggaactagacctctcccaaaactacttggccagagaaattgaggaggccaaatttttgcattttcttcccaaccttgttgagttggatttttctttcaattatgagctgcaggtctaccatgcatctataactttaccacattcactctcttcattggaaaacttgaaaattctgcgtgtcaaggggtatgtctttaaagagctgaaaaactccagtctttctgtattgcacaagcttcccaggctggaagttcttgaccttggcactaacttcataaaaattgctgacctcaacatattcaaacattttgaaaacctcaaactcatagacctttcagtgaataagatatctccttcagaagagtcaagagaagttggcttttgtcctaatgctcaaacttctgtagaccgtcatgggccccaggtccttgaggccttacactatttccgatacgatgaatatgcacggagctgcaggttcaaaaacaaagagccaccttctttcttgcctttgaatgcagactgccacatatatgggcagaccttagacttaagtagaaataacatattttttattaaaccttctgattttcagcatctttcattcctcaaatgcctcaacttatcaggaaacaccattggccaaactcttaatggcagtgaactctggccgttgagagagttgcggtacttagacttctccaacaaccggcttgatttactctactcaacagcctttgaagagctccagagtcttgaagttctggatctaagtagtaacagccactattttcaagcagaaggaattactcacatgctaaactttaccaagaaattacggcttctggacaaactcatgatgaatgataatgacatctctacttcggccagcaggaccatggaaagtgactctcttcgaattctggagttcagaggcaaccatttagatgttctatggagagccggtgataacagatacttggacttcttcaagaatttgttcaatttagaggtattagatatctccagaaattccctgaattccttgcctcctgaggtttttgagggtatgccgccaaatctaaagaatctctccttggccaaaaatgggctcaaatctttcttttgggacagactccagttactgaagcatttggaaattttggacctcagccataaccagctgacaaaagtacctgagagattggccaactgttccaaaagtctcacaacactgattcttaagcataatcaaatcaggcaattgacaaaatattttctagaagatgctttgcaattgcgctatctagacatcagttcaaataaaatccaggtcattcagaagactagcttcccagaaaatgtcctcaacaatctggagatgttggttttacatcacaatcgctttctttgcaactgtgatgctgtgtggtttgtctggtgggttaaccatacagatgttactattccatacctggccactgatgtgacttgtgtaggtccaggagcacacaaaggtcaaagtgtcatatcccttgatctgtatacgtgtgagttagatctcacaaacctgattctgttctcagtttccatatcatcagtcctctttcttatggtagttatgacaacaagtcacctctttttctgggatatgtggtacatttattatttttggaaagcaaagataaaggggtatcagcatctgcaatccatggagtcttgttatgatgcttttattgtgtatgacactaaaaactcagctgtgacagaatgggttttgcaggagctggtggcaaaattggaagatccaagagaaaaacacttcaatttgtgtctagaagaaagagactggctaccaggacagccagttctagaaaacctttcccagagcatacagctcagcaaaaagacagtgtttgtgatgacacagaaatatgctaagactgagagttttaagatggcattttatttgtctcatcagaggctcctggatgaaaaagtggatgtgattatcttgatattcttggaaaagcctcttcagaagtctaagtttcttcagctcaggaagagactctgcaggagctctgtccttgagtggcctgcaaatccacaggctcacccatacttctggcagtgcctgaaaaatgccctgaccacagacaatcatgtggcttatagtcaaatgttcaaggaaacagtctagctctctgaagaatgtcaccacctaggacatgccttgaatcga

SEQ ID NO: 22 - protein, 1050 aa, Mus musculus
MVFSMWTRKRQILIFLNMLLVSRVFGFRWFPKTLPCEVKVNIPEAHVIVDCTDKHLTEIPEGIPTNTTNLTLTINHIPSISPDSFRRLNHLEEIDLRCNCVPVLLGSKANVCTKRLQIRPGSFSGLSDLKALYLDGNQLLEIPQDLPSSLHLLSLEANNIFSITKENLTELVNIETLYLGQNCYYRNPCNVSYSIEKDAFLVMRNLKVLSLKDNNVTAVPTTLPPNLLELYLYNNIIKKIQENDFNNLNELQVLDLSGNCPRCYNVPYPCTPCENNSPLQIHDNAFNSLTELKVLRLHSNSLQHVPPTWFKNMRNLQELDLSQNYLAREIEEAKFLHFLPNLVELDFSFNYELQVYHASITLPHSLSSLENLKILRVKGYVFKELKNSSLSVLHKLPRLEVLDLGTNFIKIADLNIFKHFENLKLIDLSVNKISPSEESREVGFCPNAQTSVDRHGPQVLEALHYFRYDEYARSCRFKNKEPPSFLPLNADCHIYGQTLDLSRNNIFFIKPSDFQHLSFLKCLNLSGNTIGQTLNGSELWPLRELRYLDFSNNRLDLLYSTAFEELQSLEVLDLSSNSHYFQAEGITHMLNFTKKLRLLDKLMMNDNDISTSASRTMESDSLRILEFRGNHLDVLWRAGDNRYLDFFKNLFNLEVLDISRNSLNSLPPEVFEGMPPNLKNLSLAKNGLKSFFWDRLQLLKHLEILDLSHNQLTKVPERLANCSKSLTTLILKHNQIRQLTKYFLEDALQLRYLDISSNKIQVIQKTSFPENVLNNLEMLVLHHNRFLCNCDAVWFVWWVNHTDVTIPYLATDVTCVGPGAHKGQSVISLDLYTCELDLTNLILFSVSISSVLFLMVVMTTSHLFFWDMWYIYYFWKAKIKGYQHLQSMESCYDAFIVYDTKNSAVTEWVLQELVAKLEDPREKHFNLCLEERDWLPGQPVLENLSQSIQLSKKTVFVMTQKYAKTESFKMAFYLSHQRLLDEKVDVIILIFLEKPLQKSKFLQLRKRLCRSSVLEWPANPQAHPYFWQCLKNALTTDNHVAYSQMFKETV

SEQ ID NO: 23 - DNA, 25 bp, Homo sapiens
ctgcgctgctgcaagttacggaatg

SEQ ID NO: 24 - DNA, 25 bp, Homo sapiens
gcgcgaaatcatgacttaacgtcag

SEQ ID NO: 25 - DNA, 3310 bp, Homo sapiens
  CDS (83)..(3208)
gctcccggccgccatggcggccgcgggaattcgattctgcgctgctgcaagttacggaatgaaaaattagaacaacagaaacatggaaaacatgttccttcagtcgtcaatgctgacctgcattttcctgctaatatctggttcctgtgagttatgcgccgaagaaaatttttctagaagctatccttgtgatgagaaaaagcaaaatgactcagttattgcagagtgcagcaatcgtcgactacaggaagttccccaaacggtgggcaaatatgtgacagaactagacctgtctgataatttcatcacacacataacgaatgaatcatttcaagggctgcaaaatctcactaaaataaatctaaaccacaaccccaatgtacagcaccagaacggaaatcccggtatacaatcaaatggcttgaatatcacagacggggcattcctcaacctaaaaaacctaagggagttactgcttgaagacaaccagttaccccaaataccctctggtttgccagagtctttgacagaacttagtctaattcaaaacaatatatacaacataactaaagagggcatttcaagacttataaacttgaaaaatctctatttggcctggaactgctattttaacaaagtttgcgagaaaactaacatagaagatggagtatttgaaacgctgacaaatttggagttgctatcactatctttcaattctctttcacacgtgccacccaaactgccaagctccctacgcaaactttttctgagcaacacccagatcaaatacattagtgaagaagatttcaagggattgataaatttaacattactagatttaagcgggaactgtccgaggtgcttcaatgccccatttccatgcgtgccttgtgatggtggtgcttcaattaatatagatcgttttgcttttcaaaacttgacccaacttcgatacctaaacctctctagcacttccctcaggaagattaatgctgcctggtttaaaaatatgcctcatctgaaggtgctggatcttgaattcaactatttagtgggagaaatagcctctggggcatttttaacgatgctgccccgcttagaaatacttgacttgtcttttaactatataaaggggagttatccacagcatattaatatttccagaaacttctctaaacttttgtctctacgggcattgcatttaagaggttatgtgttccaggaactcagagaagatgatttccagcccctgatgcagcttccaaacttatcgactatcaacttgggtattaattttattaagcaaatcgatttcaaacttttccaaaatttctccaatctggaaattatttacttgtcagaaaacagaatatcaccgttggtaaaagatacccggcagagttatgcaaatagttcctcttttcaacgtcatatccggaaacgacgctcaacagattttgagtttgacccacattcgaacttttatcatttcacccgtcctttaataaagccacaatgtgctgcttatggaaaagccttagatttaagcctcaacagtattttcttcattgggccaaaccaatttgaaaatcttcctgacattgcctgtttaaatctgtctgcaaatagcaatgctcaagtgttaagtggaactgaattttcagccattcctcatgtcaaatatttggatttgacaaacaatagactagactttgataatgctagtgctcttactgaattgtccgacttggaagttctagatctcagctataattcacactatttcagaatagcaggcgtaacacatcatctagaatttattcaaaatttcacaaatctaaaagttttaaacttgagccacaacaacatttatactttaacagataagtataacctggaaagcaagtccctggtagaattagttttcagtggcaatcgccttgacattttgtggaatgatgatgacaacaggtatatctccattttcaaaggtctcaagaatctgacacgtctggatttatcccttaataggctgaagcacatcccaaatgaagcattccttaatttgccagcgagtctcactgaactacatataaatgataatatgttaaagttttttaactggacattactccagcagtttcctcgtctcgagttgcttgacttacgtggaaacaaactactctttttaactgatagcctatctgactttacatcttcccttcggacactgctgctgagtcataacaggatttcccacctaccctctggctttctttctgaagtcagtagtctgaagcacctcgatttaagttccaatctgctaaaaacaatcaacaaatccgcacttgaaactaagaccaccaccaaattatctatgttggaactacacggaaacccctttgaatgcacctgtgacattggagatttccgaagatggatggatgaacatctgaatgtcaaaattcccagactggtagatgtcatttgtgccagtcctggggatcaaagagggaagagtattgtgagtctggagctaacaacttgtgtttcagatgtcactgcagtgatattatttttcttcacgttctttatcaccaccatggttatgttggctgccctggctcaccatttgttttactgggatgtttggtttatatataatgtgtgtttagctaaggtaaaaggctacaggtctctttccacatcccaaactttctatgatgcttacatttcttatgacaccaaagacgcctctgttactgactgggtgataaatgagctgcgctaccaccttgaagagagccgagacaaaaacgttctcctttgtctagaggagagggattgggacccgggattggccatcatcgacaacctcatgcagagcatcaaccaaagcaagaaaacagtatttgttttaaccaaaaaatatgcaaaaagctggaactttaaaacagctttttacttggctttgcagaggctaatggatgagaacatggatgtgattatatttatcctgctggagccagtgttacagcattctcagtatttgaggctacggcagcggatctgtaagagctccatcctccagtggcctgacaacccgaaggcagaaggcttgttttggcaaactctgagaaatgtggtcttgactgaaaatgattcacggtataacaatatgtatgtcgattccattaagcaatactaactgacgttaagtcatgatttcgcgcaatcactagtgaattcgcggccgcctgcaggtcgaccatatgggagagctcccaacgcgttggatgcatagcttgag

SEQ ID NO: 26 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 27 - protein, 19 aa, Homo sapiens
MKESSLQNSSCSLGKETKK

SEQ ID NO: 28 - DNA, 24 bp, Mus musculus
gagagaaacaaacgttttaccttc

SEQ ID NO: 29 - DNA, 22 bp, Mus musculus
gatggcagagtcgtgacttccc

SEQ ID NO: 30 - DNA, 3220 bp, Mus musculus
  CDS (59)..(3157)
attcagagttggatgttaagagagaaacaaacgttttaccttcctttgtctatagaacatggaaaacatgccccctcagtcatggattctgacgtgcttttgtctgctgtcctctggaaccagtgccatcttccataaagcgaactattccagaagctatccttgtgacgagataaggcacaactcccttgtgattgcagaatgcaaccatcgtcaactgcatgaagttccccaaactataggcaagtatgtgacaaacatagacttgtcagacaatgccattacacatataacgaaagagtcctttcaaaagctgcaaaacctcactaaaatcgatctgaaccacaatgccaaacaacagcacccaaatgaaaataaaaatggtatgaatattacagaaggggcacttctcagcctaagaaatctaacagttttactgctggaagacaaccagttatatactatacctgctgggttgcctgagtctttgaaagaacttagcctaattcaaaacaatatatttcaggtaactaaaaacaacacttttgggcttaggaacttggaaagactctatttgggctggaactgctattttaaatgtaatcaaacctttaaggtagaagatggggcatttaaaaatcttatacacttgaaggtactctcattatctttcaataaccttttctatgtgccccccaaactaccaagttctctaaggaaactttttctgagtaatgccaaaatcatgaacatcactcaggaagacttcaaaggactggaaaatttaacattactagatctgagtggaaactgtccaaggtgttacaatgctccatttccttgcacaccttgcaaggaaaactcatccatccacatacatcctctggcttttcaaagtctcacccaacttctctatctaaacctttccagcacttccctcaggacgattccttctacctggtttgaaaatctgtcaaatctgaaggaactccatcttgaattcaactatttagttcaagaaattgcctcgggggcatttttaacaaaactacccagtttacaaatccttgatttgtccttcaactttcaatataaggaatatttacaatttattaatatttcctcaaatttctctaagcttcgttctctcaagaagttgcacttaagaggctatgtgttccgagaacttaaaaagaagcatttcgagcatctccagagtcttccaaacttggcaaccatcaacttgggcattaactttattgagaaaattgatttcaaagctttccagaatttttccaaactcgacgttatctatttatcaggaaatcgcatagcatctgtattagatggtacagattattcctcttggcgaaatcgtcttcggaaacctctctcaacagacgatgatgagtttgatccacacgtgaatttttaccatagcaccaaacctttaataaagccacagtgtactgcttatggcaaggccttggatttaagtttgaacaatattttcattattgggaaaagccaatttgaaggttttcaggatatcgcctgcttaaatctgtccttcaatgccaatactcaagtgtttaatggcacagaattctcctccatgccccacattaaatatttggatttaaccaacaacagactagactttgatgataacaatgctttcagtgatcttcacgatctagaagtgctggacctgagccacaatgcacactatttcagtatagcaggggtaacgcaccgtctaggatttatccagaacttaataaacctcagggtgttaaacctgagccacaatggcatttacaccctcacagaggaaagtgagctgaaaagcatctcactgaaagaattggttttcagtggaaatcgtcttgaccatttgtggaatgcaaatgatggcaaatactggtccatttttaaaagtctccagaatttgatacgcctggacttatcatacaataaccttcaacaaatcccaaatggagcattcctcaatttgcctcagagcctccaagagttacttatcagtggtaacaaattacgtttctttaattggacattactccagtattttcctcaccttcacttgctggatttatcgagaaatgagctgtattttctacccaattgcctatctaagtttgcacattccctggagacactgctactgagccataatcatttctctcacctaccctctggcttcctctccgaagccaggaatctggtgcacctggatctaagtttcaacacaataaagatgatcaataaatcctccctgcaaaccaagatgaaaacgaacttgtctattctggagctacatgggaactattttgactgcacgtgtgacataagtgattttcgaagctggctagatgaaaatctgaatatcacaattcctaaattggtaaatgttatatgttccaatcctggggatcaaaaatcaaagagtatcatgagcctagatctcacgacttgtgtatcggataccactgcagctgtcctgtttttcctcacattccttaccacctccatggttatgttggctgctctggttcaccacctgttttactgggatgtttggtttatctatcacatgtgctctgctaagttaaaaggctacaggacttcatccacatcccaaactttctatgatgcttatatttcttatgacaccaaagatgcatctgttactgactgggtaatcaatgaactgcgctaccaccttgaagagagtgaagacaaaagtgtcctcctttgtttagaggagagggattgggatccaggattacccatcattgataacctcatgcagagcataaaccagagcaagaaaacaatctttgttttaaccaagaaatatgccaagagctggaactttaaaacagctttctacttggccttgcagaggctaatggatgagaacatggatgtgattattttcatcctcctggaaccagtgttacagtactcacagtacctgaggcttcggcagaggatctgtaagagctccatcctccagtggcccaacaatcccaaagcagaaaacttgttttggcaaagtctgaaaaatgtggtcttgactgaaaatgattcacggtatgacgatttgtacattgattccattaggcaatactagtgatgggaagtcacgactctgccatcataaaaacacacagcttctccttacaatgaaccgaat

SEQ ID NO: 31 - protein, 1032 aa, Mus musculus
MENMPPQSWILTCFCLLSSGTSAIFHKANYSRSYPCDEIRHNSLVIAECNHRQLHEVPQTIGKYVTNIDLSDNAITHITKESFQKLQNLTKIDLNHNAKQQHPNENKNGMNITEGALLSLRNLTVLLLEDNQLYTIPAGLPESLKELSLIQNNIFQVTKNNTFGLRNLERLYLGWNCYFKCNQTFKVEDGAFKNLIHLKVLSLSFNNLFYVPPKLPSSLRKLFLSNAKIMNITQEDFKGLENLTLLDLSGNCPRCYNAPFPCTPCKENSSIHIHPLAFQSLTQLLYLNLSSTSLRTIPSTWFENLSNLKELHLEFNYLVQEIASGAFLTKLPSLQILDLSFNFQYKEYLQFINISSNFSKLRSLKKLHLRGYVFRELKKKHFEHLQSLPNLATINLGINFIEKIDFKAFQNFSKLDVIYLSGNRIASVLDGTDYSSWRNRLRKPLSTDDDEFDPHVNFYHSTKPLIKPQCTAYGKALDLSLNNIFIIGKSQFEGFQDIACLNLSFNANTQVFNGTEFSSMPHIKYLDLTNNRLDFDDNNAFSDLHDLEVLDLSHNAHYFSIAGVTHRLGFIQNLINLRVLNLSHNGIYTLTEESELKSISLKELVFSGNRLDHLWNANDGKYWSIFKSLQNLIRLDLSYNNLQQIPNGAFLNLPQSLQELLISGNKLRFFNWTLLQYFPHLHLLDLSRNELYFLPNCLSKFAHSLETLLLSHNHFSHLPSGFLSEARNLVHLDLSFNTIKMINKSSLQTKMKTNLSILELHGNYFDCTCDISDFRSWLDENLNITIPKLVNVICSNPGDQKSKSIMSLDLTTCVSDTTAAVLFFLTFLTTSMVMLAALVHHLFYWDVWFIYHMCSAKLKGYRTSSTSQTFYDAYISYDTKDASVTDWVINELRYHLEESEDKSVLLCLEERDWDPGLPIIDNLMQSINQSKKTIFVLTKKYAKSWNFKTAFYLALQRLMDENMDVIIFILLEPVLQYSQYLRLRQRICKSSILQWPNNPKAENLFWQSLKNVVLTENDSRYDDLYIDSIRQY